SEQUENCE LISTING (36 sequences)

SEQ ID NO: 1 - protein, 106 aa, Unknown
  Rabbit
GQPAVTPSVILFPPSSEELKDNKATLVCLINDFYPRTVKVNWKADGNSVTQGVDTTQPSKQSNNKYAASSFLSLSANQWKSYQSVTCQVTHEGHTVEKSLAPAECS

SEQ ID NO: 2 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 3 - protein, 106 aa, Artificial Sequence
  Synthetic
GQPAVTPSVTLFPPSSEELQANKATLVCLINDFYPRTVKVNWKADGNSVKAGVETTKPSKQSNNKYAASSYLSLTPEQWKSYQSVTCQVTHEGHTVEKSLAPAECS

SEQ ID NO: 4 - protein, 106 aa, Artificial Sequence
  Synthetic
GQPAVTPSVTLFPPSSEELQANKATLVCLINDFYPRTVKVNWKADGNSVKAGVETTTPSKQSNNKYAASSYLSLTPEQWKSYQSVTCQVTHEGHTVEKSLAPAECS

SEQ ID NO: 5 - protein, 106 aa, Artificial Sequence
  Synthetic
GQPAVTPSVTLFPPSSEELQANKATLVCLINDFYPRTVKVNWKADGNSVKVGVETTKPSKQSNNKYAASSYLSLTPEQWKSYQSVTCQVTHEGHTVEKSLAPAECS

SEQ ID NO: 6 - protein, 104 aa, Homo sapiens
PKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 7 - protein, 104 aa, Unknown
  Rabbit FLC
PAVTPSVILFPPSSEELKDNKATLVCLINDFYPRTVKVNWKADGNSVTQGVDTTQPSKQSNNKYAASSFLSLSANQWKSYQSVTCQVTHEGHTVEKSLAPAECS

SEQ ID NO: 8 - protein, 106 aa, Artificial Sequence
  Synthetic
GQPAVTPTVTLFPPSSEELKDNKATLVCLISDFYPGAVTVNWKADGNSVTQGVETTKPSKQSNNKYAASSYLSLSANQWKSYQSVTCQVTHEGHTVEKSLAPTECS

SEQ ID NO: 9 - protein, 104 aa, Homo sapiens
RSFFSFLGEAFDGARDMWRAYSDMREANYIGSDKYFHARGNYDAAKRGPGGAWAAEVITDARENIQRFFGHGAEDSLADQAANEWGRSGKDPNHFRPAGLPEKY

SEQ ID NO: 10 - protein, 104 aa, Unknown
  Rabbit
QRWFSFIGEATQGAWDMWRAYSDMREANYINADKYFHARGNYDAAQRGPGGVWAAKVISDAREDLQRLMGHGAEDSMADQAANEWGRSGKDPNHFRPKGLPDKY

SEQ ID NO: 11 - protein, 104 aa, Artificial Sequence
  Synthetic
RSWFSFIGEATDGARDMWRAYSDMREANYIGSDKYFHARGNYDAAKRGPGGVWAAEVISDARENLQRLMGHGAEDSLADQAANEWGRSGKDPNHFRPAGLPEKY

SEQ ID NO: 12 - protein, 107 aa, Homo sapiens [sequence omitted]

SEQ ID NO: 13 - protein, 104 aa, Unknown
  Rabbit
GDPVAPSVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 14 - protein, 104 aa, Artificial Sequence
  Synthetic
GDPVAPSVFIFPPAADQVATGTVSIVCLANKYYPDVTVTWEVDGTTQTTGIQNSVTEQNSADSTYSLSSTLTLTSTQYNSHKEYTCKVTQGTTSVVQSFNRGEC

SEQ ID NO: 15 - protein, 323 aa, Unknown
  Rabbit
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 16 - protein, 323 aa, Artificial Sequence
  Synthetic
GQPKAPSVFPLAPSSKDTPSSTVTLGCLVKGYLPEPVTVTWNSGTLTNGVRTFPSVRQSSGLYSLSSVVSVTSSSQPVICNVAHPATNTKVDKKVAPSTCSKPTCPPPELLGGPSVFIFPPKPKDTLMISRTPEVTCVVVDVSQDDPEVKFTWYINNEQVRTARPPLREQQFNSTIRVVSTLPIAHQDWLRGKEFKCKVHNKALPAPIEKTISKARGQPLEPKVYTMGPPRDELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 17 - protein, 323 aa, Artificial Sequence
  Synthetic
GQPKAPSVFPLAPCCGDTPSSTVTLGCLVKGYLPEPVTVTWNSGTLTNGVRTFPSVRQSSGLYSLSSVVSVTSSNFPVTCNVAHPATNTKVDKTVERSTCSKPTCPPPELLGGPSVFIFPPKPKDTLMISRTPEVTCVVVDVSQDDPEVQFTWYINNEQVRTARPPLREQQFNSTIRVVSTLPIVHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 18 - protein, 323 aa, Artificial Sequence
  Synthetic
GQPKAPSVFPLAPCCGDTPSSTVTLGCLVKGYLPEPVTVTWNSGTLTNGVRTFPSVRQSSGLYSLSSVVSVTSSNFPVTCNVAHPATNTKVDKTVERSTCSKPTCPPPPVAGGPSVFIFPPKPKDTLMISRTPEVTCVVVDVSQDDPEVQFTWYINNEQVRTARPPLREQQFNSTIRVVSTLPIVHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 19 - protein, 323 aa, Artificial Sequence
  Synthetic
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFKWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKSGKAEDNYNTTPAVLDSDGSYFLYSKLSVPTSEWQRGDIFTCSVMHEALHNRFTQKSISRSPGK

SEQ ID NO: 20 - protein, 323 aa, Artificial Sequence
  Synthetic
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPSPEFLGGPSVFIFPPKPKDTLMISRTPEVTCVVVDVSQEDPEVQFTWYINNEQVRTARPPLREQQFNSTIRVVSTLPIAHQDWLRGKEFKCKVHNKALPSSIEKTISKARGQPLEPKVYTMGPPQEELSSRSVSLTCMINGFYPSDISVEWEKNGKAEDNYKTTPAVLDSDGSYFLYSRLSVPTSEWQEGDVFTCSVMHEALHNHYTQKSISRSPGK

SEQ ID NO: 21 - protein, 323 aa, Unknown; identical to SEQ ID NO: 15
  Rabbit

SEQ ID NO: 22 - protein, 323 aa, Artificial Sequence
  Synthetic
GQPKAPSVFPLAPSSKDTPSSTVTLGCLVKGYLPEPVTVTWNSGTLTNGVRTFPSVRQSSGLYSLSSVVSVTSSSQPVICNVAHPATNTKVDKKVAPSTCDKTHCPPPELLGGPSVFIFPPKPKDTLMISRTPEVTCVVVDVSQDDPEVKFTWYINNEQVRTARPPLREQQFNSTIRVVSTLPIAHQDWLRGKEFKCKVHNKALPAPIEKTISKARGQPLEPKVYTMGPPRDELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 23 - protein, 321 aa, Artificial Sequence
  Synthetic
GQPKAPSVFPLAPCCGDTPSSTVTLGCLVKGYLPEPVTVTWNSGTLTNGVRTFPSVRQSSGLYSLSSVVSVTSSNFPVTCNVAHPATNTKVDKTVERKCCVECPPPPVAGGPSVFIFPPKPKDTLMISRTPEVTCVVVDVSQDDPEVQFTWYINNEQVRTARPPLREQQFNSTIRVVSTLPIVHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 24 - protein, 104 aa, Artificial Sequence; identical to SEQ ID NO: 11
  Synthetic

SEQ ID NO: 25 - protein, 175 aa, Unknown
  Rabbit ferritin
MTSQIRQNYSPEVEAAVNHLVNLHLRASYTYLSLGFYFDRDDVALEGVSHFFRELAEEKREAAERLLKMQNQRGGRALFQDVQKPSQDEWGKTLNAMEAALALEKNLNQALLDLHALGSAHTDPHLCDFLENHFLDEEVKLLKKMGDHLTNIRRLSGPQASLGEYLFERLTLKHD

SEQ ID NO: 26 - protein, 175 aa, Homo sapiens
  misc_feature: Human ferritin
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 27 - protein, 306 aa, Artificial Sequence
  Synthetic
GQPAVTPTVTLFPPSSEELKDNKATLVCLISDFYPGAVTVNWKADGNSVTQGVETTKPSKQSNNKYAASSYLSLSANQWKSYQSVTCQVTHEGHTVEKSLAPTECSGGGGSGGGGSGGGGSGGGGSGGGGSMTSQIRQNYSPEVEAAVNHLVNLHLRASYTYLSLGFYFDRDDVALEGVSHFFRELAEEKREAAERLLKMQNQRGGRALFQDVQKPSQDEWGKTLNAMEAALALEKNLNQALLDLHALGSAHTDPHLCDFLENHFLDEEVKLLKKMGDHLTNIRRLSGPQASLGEYLFERLTLKHD

SEQ ID NO: 28 - protein, 106 aa, Artificial Sequence; identical to SEQ ID NO: 8
  Synthetic

SEQ ID NO: 29 - protein, 25 aa, Artificial Sequence
  Synthetic
GGGGSGGGGSGGGGSGGGGSGGGGS

SEQ ID NO: 30 - protein, 175 aa, Unknown; identical to SEQ ID NO: 25
  Rabbit ferritin

SEQ ID NO: 31 - protein, 5 aa, Artificial Sequence
  Synthetic
GGGGS

SEQ ID NO: 32 - protein, 10 aa, Artificial Sequence
  Synthetic
HHHHHHKHHH

SEQ ID NO: 33 - protein, 41 aa, Artificial Sequence
  Synthetic
STNNNEEEKSRLLEKENRELEKIIAEKEERVSELRHQLQSR

SEQ ID NO: 34 - protein, 41 aa, Artificial Sequence
  Synthetic
SVNQASTSRLEGLQSENHRLRMKITELDKDLEEVTMQLQDT

SEQ ID NO: 35 - protein, 306 aa, Artificial Sequence; identical to SEQ ID NO: 27
  Synthetic

SEQ ID NO: 36 - protein, 241 aa, Artificial Sequence
  Synthetic
STNNNEEEKSRLLEKENRELEKIIAEKEERVSELRHQLQSRGGGGSGGGGSGGGGSGGGGSGGGGSMTSQIRQNYSPEVEAAVNHLVNLHLRASYTYLSLGFYFDRDDVALEGVSHFFRELAEEKREAAERLLKMQNQRGGRALFQDVQKPSQDEWGKTLNAMEAALALEKNLNQALLDLHALGSAHTDPHLCDFLENHFLDEEVKLLKKMGDHLTNIRRLSGPQASLGEYLFERLTLKHD

(19) United States
(12) Patent Application Publication
(10) Pub. No.: US 2023/0054648 A1
(43) Pub. Date: Feb. 23, 2023

(54) CHIMERIC IMMUNOGENS AND METHODS FOR MAKING POLYCLONAL ANTIBODIES AGAINST SPECIFIC EPITOPES

(71) Applicant: Agilent Technologies, Inc., Santa Clara, CA (US)

(72) Inventors: Jesper KUHNAU, Allerød (DK); Kim Krighaar RASMUSSEN, Copenhagen (DK)

(21) Appl. No.: 17/735,780

(22) Filed: May 3, 2022

Related U.S. Application Data
(60) Provisional application No. 63/183,616, filed on May 3, 2021.

(51) Int. Cl. C07K 16/00; C07K 16/42; A61K 31/7088; C12N 15/86; C12N 15/11

(52) U.S. Cl. CPC C07K 16/00; C07K 16/4283; A61K 31/7088; C12N 15/86; C12N 15/11; A61K 38/00

(57) ABSTRACT

In alternative embodiments, provided are chimeric immunogens or antigens, and methods for making and using them, including methods for making and obtaining polyclonal antibodies specific for selected epitopes. In alternative embodiments, provided are methods for generating an epitope-specific antibody response in a rabbit, wherein the immune response comprises generation of rabbit antibodies specifically against (or that specifically bind to) at least one human epitope, and the method comprises administering to a rabbit a sufficient amount of a chimeric or recombinant polypeptide to generate the epitope-specific antibody response. In alternative embodiments, provided are chimeric or recombinant polypeptides comprising: a ferritin polypeptide having conjugated or attached thereto by or via a substantially non-immunogenic linker an immunogenic peptide or polypeptide.

### RELATED APPLICATIONS

This U.S. utility patent application claims the benefit of priority under 35 U.S.C. § 119(e) to U.S. Provisional Patent Application Ser. No. 63/183,616, May 3, 2021. The aforementioned application is expressly incorporated herein by reference in its entirety and for all purposes.

### TECHNICAL FIELD

This invention generally relates to immunology and immunoassays. In alternative embodiments, provided are chimeric immunogens, and methods for making and using them, including methods for making and obtaining polyclonal antibodies specific for selected epitopes. In alternative embodiments, provided are methods for generating an epitope-specific antibody response in a rabbit, wherein the immune response comprises generation of rabbit antibodies specifically against (or that specifically bind to) at least one human epitope, and the method comprises administering to a rabbit a sufficient amount of a chimeric or recombinant polypeptide to generate the epitope-specific antibody response. In alternative embodiments, provided are chimeric or recombinant polypeptides comprising: a ferritin polypeptide having conjugated or attached thereto by or via a substantially non-immunogenic linker an immunogenic peptide or polypeptide.

### BACKGROUND

Polyclonal antibodies have a diverse reactivity towards multiple epitopes ensuring a robust reaction even in the face of diversity of the target or environmental changes. To obtain polyclonal antibodies, an animal is immunized with a protein, a protein fragment or a mix thereof, after which the humoral immune system selects antibody producing B-cell clones for expansion and maturation. At later stages these B-cells will further diversify through mutagenesis and selection of high affinity immunoglobulin genes. While the immune system has a basic capability to make antibodies against almost any foreign protein, it is known that some epitopes are dominant and that B-cell clones producing antibody that recognizes these “dominant” epitopes will take over the immune response. This means that a standard polyclonal antibody is biased towards some epitopes and may lack reactivity towards other epitopes.

In principle, immunization can be by using a single (for example, linear) epitope using a single peptide or a mix of peptides. Peptides may, however, not have the same three dimensional (3D) structure as the protein from which they were derived, thus causing generation of antibodies with less or no affinity to the protein. Peptides (particularly those that are not dominant epitopes) are often too small to elicit an immune response on their own and either need to be built into a larger structure or need to be dependent on co-stimulation with a more immunogenic component to stimulate the immunized animal to generate antibodies against other than the dominant epitopes preferred by the humoral response.

It is possible to tolerize against non-selected epitopes using various techniques such as neonatal, drug-induced, masking subtractive immunization or high zone tolerization methods (see for example, U.S. Pat. Nos. 7,598,030; 8,133,744) but tolerization may be a leaky process where antibody clones against non-selected epitopes continuous to show up at some level; and it has been suggested to use combinations to get higher efficiency. In all cases, tolerization means that besides the standard immunization additional procedures are needed as part of the process increasing complexity and cost.

Antibody for commercial use is purified from the immunized animal's serum. Even though total immunoglobulin may be extracted it is frequently necessary to purify the antibody further either by subtracting unwanted reactivity (adsorption purification) or by specifically selecting desired reactivity (affinity purification).

It would be advantageous to be able to specify which specific epitopes the polyclonal antibody will recognize without having to add additional steps to the immunization and/or purification procedure. For example, it would be advantageous to eliminate the need for costly and time-consuming adsorption and/or affinity purification steps.

### SUMMARY

In alternative embodiments, provided are chimeric or recombinant polypeptides comprising:

(a) a polypeptide derived from a first species, and

(b) at least one heterologous amino acid sequence or amino acid residue derived from at least a second species,

wherein the at least one heterologous amino acid sequence or amino acid residue derived from the second or additional species is inserted into, joined to, created in, or replaced for or substituted for a portion of the amino acid sequence of the polypeptide derived from the first species,

and the amino acid sequence of the chimeric or recombinant polypeptide is substantially comprised of amino acid sequence derived from the first species,

and the amino acid sequence from the second species when inserted into, joined to, created in, or replaced for or substituted for a portion of the amino acid sequence of the polypeptide derived from the first species generates, forms or creates at least one new epitope on the polypeptide derived from the first species that is capable of generating a humoral antibody response by the first species specific for the at least one new epitope when the chimeric or recombinant polypeptide is administered to the first species,

wherein when the chimeric or recombinant polypeptide is used to generate a humoral immune response from an animal of the first species, the polyclonal antibodies so generated in the first species substantially only specifically bind to the at least one new epitope and do not specifically bind to, or substantially do not specifically bind to, or only bind with low affinity to, the polypeptide derived from the first species lacking the at least one new epitope or epitopes created, formed or generated by the at least one heterologous amino acid sequence or amino acid residue derived from the second or additional species inserted into, joined to, created in, or replaced for or substituted for a portion of the polypeptide derived from a first species. In other words, there may be some low affinity and/or non-specific binding of antibodies newly generated in the first species to proteins to polypeptides from the first species which do not have contained therein the protein sequence forming the at least one new epitope from the second species.

Another possible scenario can be seen when using protein domains such as the constant domain of a light chain for immunization (the constant domain as the polypeptide derived from the first species), where there may be an antibody response in the first species towards surfaces not normally exposed (for example, not normally exposed when the protein is normally folded, or in its native three dimensional (3D) structure) in a physiologic environment), for example, a surface in a constant region not normally exposed is the linker region (the region linking the variable and the constant domains) between constant and variable domains in the lambda light chain and the C-terminal cysteine that is normally linked to the heavy chain in intact IgG. That is, when a domain is removed from its normal context it is possible to have a humoral response to the now exposed (in the chimeric or recombinant polypeptide) surface or surfaces. Significantly, while this may cause antibody generation towards a newly exposed homolog sequence (for example, a rabbit homolog sequence such as a rabbit constant domain of lambda light chain) in an immunogen, it should not direct a humoral response that recognizes normal (normally folded) light chains because these regions (surfaces not normally exposed) will be hidden or are folded in the native 3D structure.

In alternative embodiments of chimeric or recombinant polypeptides as provided herein;

the polypeptide derived from the second species is a homologue of the polypeptide derived from the first species;

the amino acid sequence from the at least one second species is homologous to the first species, and the at least one homologous second species sequence that is inserted into, joined to, created in, or replaced for or substituted for a portion of the amino acid sequence of the polypeptide derived from the first species replaces all or substantially all of a structurally homologous section or portion of the amino acid sequence of the polypeptide derived from the first species;

the amino acid sequence from the at least one second species is homologous to the first species, and the at least one homologous second species sequence that is inserted into, joined to, created in, or replaced for or substituted for a portion of the amino acid sequence of the polypeptide derived from the first species is structurally homologous to an amino acid sequence of the polypeptide derived from the first species;

a homologue of a first species has at least about 25% to 99% sequence identity to its homologue in the second species;

the homologue of the first species has substantially the same secondary and/or tertiary structure as its homologue in the second species;

a homologue of a first species has at least about 25% to 99% sequence identity to its homologue in the second species and has substantially the same secondary and/or tertiary structure as its homologue in the second species;

a homologue of a first species has at least about 50% sequence identity to its homologue in the second species; or, a homologue of a first species has at least about 70% sequence identity to its homologue in the second species; or, a homologue of a first species has at least about 80% sequence identity to its homologue in the second species; or, a homologue of a first species has at least about 90% sequence identity to its homologue in the second species;

the first polypeptide and the second polypeptide have a Z score of from about 2 to about 8 when aligned using distance matrix alignment; or, the first polypeptide and the second polypeptide have a Z score of at least 8 when aligned using distance matrix alignment;

the polypeptide derived from the first species and its homologue polypeptide from the second species are antibodies; or, the polypeptide derived from the first species and the at least one heterologous amino acid sequence derived from the second species are derived from an antibody heavy chain or an antibody light chain;

the antibody heavy chain is an IgM, IgG, IgA or IgE isotype heavy chain, or the light chain is a kappa or a lambda light chain;

the first species is a mammalian species; the second species is a mammalian species; or, the first species is a species of the order Galliformes or the genus Phasianidae and the second species is a mammalian species; or, the first species is a rabbit, a murine species, a sheep, a goat, a pig, a cow a horse or a chicken; and, the second species is a human; or, the murine specie is a rat or a mouse;

at least about 80% to about 99% of the amino acid sequence of the chimeric or recombinant polypeptide is amino acid sequence derived from the first species, and/or between about 1% to about 20% of the amino acid sequence of the chimeric or recombinant polypeptide is amino acid sequence derived from the at least one second species;

one, two three, four, five, six, seven or eight or more new epitopes are inserted into, joined to, created in, or replaced for or substituted for a portion of the polypeptide derived from the first species;

the at least one new epitope comprises an epitope derived from a hidden surface of an antibody light chain, wherein the hidden surface is only exposed when the antibody light chain is free and not part of an IgG molecule comprising both light and heavy chains;

the epitope generated, created or formed by the at least one heterologous amino acid sequence derived from the at least one second species is designed by:
(a) aligning the sequence of the polypeptide derived from the first species with its homologue polypeptide from the second species,
(b) determining one or more amino acid sequence differences between the polypeptide derived from the first species and its homologue polypeptide from the second species,
(c) selecting at least one amino acid sequence difference between the polypeptide derived from the first species and its homologue polypeptide from the second species, and
(d) modifying the sequence of the polypeptide derived from the first species to match or be the same as the selected at least one amino acid sequence from the homologue polypeptide of the second species;

selecting at least one amino acid sequence difference between the polypeptide derived from the first species and its homologue polypeptide from the second species comprises highlighting the determined one or more amino acid sequence differences between the polypeptide derived from the first species and its homologue polypeptide from the second species on a 3D model or structure of the polypeptide from the second species, and selecting at least one amino acid sequence difference in or on an exposed or outer surface of the polypeptide;

the amino acid sequence from the at least one second species inserted into, joined to, created in, or replaced for or substituted for a portion of the amino acid sequence of the polypeptide derived from the first species comprises: a sequence present in human IgG3 and not human IgG1, IgG2 or IgG4, or rabbit IgG; a sequence present in human IgG1 and not human IgG2, IgG3 or IgG4, or rabbit IgG; a sequence present in human IgG2 and not human IgG1, IgG3 or IgG4, or rabbit IgG; or, a sequence present in human IgG4 and not human IgG1, IgG2 or IgG3, or rabbit IgG;

the chimeric or recombinant polypeptide is made by a method further comprising removing one or more new epitopes from the at least one heterologous amino acid sequence derived from the second or additional species after the one or more new epitopes was inserted into, joined to, created in, or replaced for or substituted for a portion of the amino acid sequence of the polypeptide derived from the first species, for example, as illustrated in FIG. 8;

at least two or more different heterologous amino acid sequences or amino acid residues are inserted into, joined to, created in, or replaced for or substituted for a portion of the amino acid sequence of the polypeptide derived from the first species, and optionally the at least two or more different heterologous amino acid sequences or amino acid residues are from different animal species, and optionally at least one of the at least two or more different heterologous amino acid sequences or amino acid residues is derived from a human and at least one of the at least two or more different heterologous amino acid sequences or amino acid residues is derived from a non-human or an animal species, for example, as illustrated in FIG. 9;

at least one of the heterologous amino acid sequences or amino acid residues comprises an artificial epitope not derived from the at least a second species, for example, as illustrated in FIG. 10;

at least one of the heterologous amino acid sequences or amino acid residues comprises an epitope initially derived from the at least a second species that is immunologically silent in the first species (is unable to generate an antibody response in the first species) but is modified to be an immunologically active epitope capable of generating an antibody response against it by the first species, for example, as illustrated in FIG. 10;

at least one new epitope in the heterologous amino acid sequences or amino acid residues is modified such that antibodies generated by the first species to the modified new epitope bind less strongly or slower than a comparable unmodified new epitope, for example, as illustrated in FIG. 11; and/or

the chimeric or recombinant polypeptide further comprises at least one new epitope derived from an at least second species that is not homologous to the first species, and the at least one new epitope of capable of generating antibodies against it in the first species, for example, as illustrated in FIG. 12.

In alternative embodiments, provided are recombinant polypeptides comprising a portion of a first polypeptide from a first species and at least one portion of a second polypeptide from a second species, wherein the at least one portion of the second polypeptide is a homologue of the first polypeptide, and wherein the at least one homologous portion of the second polypeptide comprises an epitope which is not present in the first polypeptide.

In alternative embodiments, of recombinant polypeptides as provided herein:

the portion of the at least one second polypeptide is present at the location of, or substantially at the location of, a homologous portion of the first polypeptide, and has replaced or substantially replaced the homologous portion of the first polypeptide;

the recombinant polypeptides comprise at least a portion of a second polypeptide and at least a portion of a third polypeptide, each being a homologue of different sequences of the first species, and wherein the portion of the second and the portion of the third polypeptide each comprises an epitope which is not present in the first polypeptide;

the first polypeptide and the second polypeptide have similar, or substantially the same, 3D structures;

the first polypeptide and the second polypeptide have between about 25% and about 95% amino acid identity; or the first polypeptide and the second polypeptide have at least about 25% amino acid identity; or, the first polypeptide and the second polypeptide have at least about 50% amino acid identity; or, the first polypeptide and the second polypeptide have at least about 70% amino acid identity; or, the first polypeptide and the second polypeptide have at least about 90% amino acid identity;

the first polypeptide and the second polypeptide have a Z score of from about 2 to about 8 when aligned using distance matrix alignment; or, the first polypeptide and the second polypeptide have a Z score of at least 8 when aligned using distance matrix alignment;

at least one sequence in the first polypeptide is removed and replaced by at least one epitope formed by a homologous portion of the second polypeptide;

at least one sequence that has been removed from the first polypeptide comprises a sequence that is present in another member of a family from which the first polypeptide and the second polypeptide belong;

at least one sequence that has been removed comprises a sequence that is present in a domain in another member of a family to which the first polypeptide and the second polypeptide belong;

the at least one sequence that is replaced by a sequence comprising an epitope that is specifically recognized by a monoclonal antibody;

the at least one epitope that has been replaced is replaced by a sequence comprising an epitope that results in at least one paratope (antigen binding site) subtype on the generated antibody;

the at least one epitope that has been replaced is replaced by a sequence comprising an epitope that is a dominant, or more dominant, epitope;

the at least one epitope that has been replaced is replaced by a sequence comprising an epitope that is a weak epitope or weaker epitope, or an epitope that elicits a weak humoral response in the first species leading to relatively less titer of antibody,

in alternative embodiments, one or more parts (or epitopes) of the sequence from the second species to be inserted into the first species polypeptide is first modified or changed to be the same as or more similar to a sequence from the first species, where this ensures that only one or some of the epitopes originally or natively present in the second sequence remain present in the final recombinant or chimeric polypeptide; this can make the generated polyclonal antibody more specific for selected targets (or epitopes) (for example, by decreasing the number of epitopes present in the transferred second species); or, this can change the characteristic or property of the generated polyclonal antibody by removing the possibility that highly hydrophobic paratopes will be in the generated antibody;

the epitope in the second polypeptide is modified to reduce the affinity of an antibody generated by the first species which specifically recognizes the epitope as compared to an unmodified epitope;

the recombinant polypeptide comprises a portion from a third polypeptide from a third species which comprises an epitope which is not present in the first polypeptide or the second polypeptide;

at least one epitope that is present in another member of a family from which the first polypeptide and the second polypeptide belong has been incorporated into the recombinant polypeptide;

at least one epitope that is present in a domain in another member of a family from which the first polypeptide and the second polypeptide belong is incorporated into the recombinant polypeptide;

the epitope from the second polypeptide is modified to increase the affinity of an antibody which specifically recognizes the epitope from the second polypeptide, or to generate an affinity to the epitope from the second polypeptide by an antibody which specifically recognizes the epitope;

the first species is rabbit and the second species is human; or the first polypeptide is a rabbit antibody light chain and the second polypeptide is a human antibody light chain; and/or

the recombinant polypeptide is administered to the first species, the epitope is capable of generating the production of antibodies which specifically bind to the epitope in the second polypeptide but which do not specifically bind to the first polypeptide.

In alternative embodiments, provided are recombinant nucleic acids encoding a chimeric or recombinant polypeptide as provided herein.

In alternative embodiments, of a recombinant nucleic acid as provided herein:

the recombinant nucleic acid is or comprises a DNA or an RNA molecule, wherein optionally the RNA is an mRNA molecule, or the recombinant nucleic acid comprises synthetic or modified nucleotides that can be utilized by cell machinery to make a polypeptide;

the recombinant nucleic acid further comprises and is operatively linked to a transcriptional regulatory element, and optionally the transcriptional regulatory element comprises a promoter, and optionally the promoter is an inducible promoter or a constitutive promoter;

the recombinant nucleic acid further comprises sequence encoding an additional protein or peptide moiety or domain;

the additional protein or peptide moiety or domain comprises a purification moiety or domain to aid in the purification or isolation of the chimeric or recombinant antibody encoded by the recombinant nucleic acid;

the additional protein or peptide moiety or domain comprises a histidine (poly-his) tag or a maltose binding protein; and/or

the recombinant nucleic acid further comprises sequence encoding a protease cleavage site positioned between the purification moiety or domain and the sequence encoding the chimeric or recombinant antibody, and optionally the protease cleavage site is a Tobacco Etch Virus (TEV) protease cleavage site.

In alternative embodiments, provided are expression cassettes, vectors, recombinant viruses, artificial chromosomes, cosmids or plasmids comprising a recombinant nucleic acid as provided herein.

In alternative embodiments, provided are cells comprising a chimeric or recombinant polypeptide as provided herein, a recombinant nucleic acid as provided herein, or an expression cassette, vector, recombinant virus, artificial chromosome, cosmid or plasmid as provided herein; and optionally the cell is a bacterial, fungal, mammalian, yeast, insect or plant cell.

In alternative embodiments, provided are methods for generating a polyclonal antibody, or for generating a polyclonal immune serum, that is specific for or specifically binds to an epitope, the method comprising:
(a) administering to or immunizing a subject with a chimeric or recombinant polypeptide as provided herein,
(b) administering to a subject a recombinant nucleic acid as provided herein or an expression cassette, vector, recombinant virus, artificial chromosome, cosmid or plasmid as provided herein, or
(c) administering to a subject a cell as provided herein,

wherein the subject is the species from which the first polypeptide as provided herein is derived, or the subject is the species from which the portion of the first polypeptide as provided herein is from, and the epitope is derived from the species from which the second polypeptide as provided herein is derived, or the epitope is derived from the species which the portion of the second polypeptide as provided herein is from.

In alternative embodiments of methods as provided herein:

the subject is a mammal or an avian species; or, the subject is a rabbit, a murine species, a sheep, a goat, a pig, a cow a horse or a chicken; and optionally the murine specie is a rat or a mouse;

the recombinant nucleic acid is an RNA or a DNA construct;

the chimeric or recombinant polypeptide is generated by expressing a recombinant nucleic acid as provided herein, or an expression cassette, vector, recombinant virus, artificial chromosome, cosmid or plasmid as provided herein, in a cell;

the cell is a bacterial, fungal, mammalian, yeast, insect or plant cell;

the method further comprises substantially isolating or purifying the chimeric or recombinant polypeptide before the administering to or immunizing the mammal;

the isolating or purifying comprising use of hydrophobic interaction chromatography (HIC), ion exchange chromatography (IEC), size exclusion chromatography (SEC), affinity purification, absorption purification or any combination thereof;

the administering of step (a), (b) or (c) is repeated between two and twenty times, or is repeated 2, 3, 4, 5, 6, 7, 8, 9 or 10 times, or is repeated at intervals of once every 2 to 20 weeks or 3 to 16 weeks;

the method generates a polyclonal antibody or a polyclonal immune serum that substantially lack antibodies that are not specific for or do not specifically bind to the epitope;

the method generates a polyclonal antibody or a polyclonal immune serum that substantially comprise antibodies that are not specific for or do not specifically bind to a misfolded form of the epitope;

at least one sequence in the first polypeptide is removed and replaced by an epitope formed by a portion of the second polypeptide;

at least one epitope in the sequence from the second polypeptide is replaced by an epitope that is present in another member of a family from which the first polypeptide and the second polypeptide belong;

at least one epitope in the sequence from the second polypeptide is replaced by a sequence comprising an epitope that is present in a domain in another member of a family to which the first polypeptide and the second polypeptide belong;

at least one epitope in the second polypeptide that is specifically recognized by a monoclonal antibody is replaced by the corresponding sequence derived from the first polypeptide;

at least one sequence in the second polypeptide comprising an epitope that results in (or generates) at least one paratope subtype is replaced with the corresponding sequence from the first polypeptide;

at least one sequence in the second polypeptide comprising a dominant epitope is replaced by the corresponding sequence derived from the first polypeptide;

at least one sequence in the second polypeptide comprising a weak epitope, or an epitope that elicits a weak humoral response leading to relatively less titer of antibody, is replaced by the corresponding sequence derived from the first polypeptide;

the epitope in the second polypeptide is modified to reduce the affinity of an antibody which specifically recognizes the epitope;

the recombinant polypeptide comprises a portion from a third polypeptide from a third species which comprises an epitope which is not present in the first polypeptide or the second polypeptide;

at least one epitope that is present in another member of a family from which the first polypeptide and the second polypeptide belong has been incorporated into the recombinant polypeptide;

at least one epitope that is present in a domain in another member of a family from which the first polypeptide and the second polypeptide belong is incorporated into the recombinant polypeptide; and/or

the epitope from the second polypeptide is modified to increase the affinity of an antibody which specifically recognizes the epitope from the second polypeptide, or to generate an affinity to the epitope from the second polypeptide by an antibody which specifically recognizes the epitope.

In alternative embodiments, provided are chimeric or recombinant polypeptides as provided herein; a nucleic acid as provided herein; an expression cassette, vector, recombinant virus, artificial chromosome, cosmid or plasmid as provided herein; or, a cell as provided herein, for use in generating a polyclonal antibody, or for generating a polyclonal immune serum, that is specific for or specifically binds to an epitope.

In alternative embodiments, provided are uses of: (a) a chimeric or recombinant polypeptide as provided herein; (b) a nucleic acid as provided herein; (c) an expression cassette, vector, recombinant virus, artificial chromosome, cosmid or plasmid as provided herein; or, (d) a cell as provided herein, for generating a polyclonal antibody, or for generating a polyclonal immune serum, that is specific for or specifically binds to an epitope.

In alternative embodiments, provided are chimeric or recombinant polypeptides comprising: a ferritin polypeptide having conjugated or attached thereto by or via a substantially non-immunogenic linker an immunogenic peptide or polypeptide, wherein the immunogenic peptide or polypeptide comprises a chimeric or recombinant polypeptide as provided herein, and the ferritin polypeptide is or is derived from the first species. In alternative embodiments of these chimeric or recombinant polypeptides:

the ferritin polypeptide is folded as a a helical bundle that assembles into a ball-like structure containing 24 copies of the ferritin polypeptide,

the substantially non-immunogenic linker comprises a poly-G linker or poly-(GGGGS) linker (SEQ ID NO:31), and optionally the poly-(GGGGS) linker (SEQ ID NO:31) comprises or consists of a (GGGGS)₅ (SEQ ID NO:29) linker,

the ferritin polypeptide carries at least one His(6)-Lys-His(3) (SEQ ID NO:32) moiety, or a plurality of His(6)-Lys-His(3) (SEQ ID NO:32) moieties;

after the linker and/or the His(6)-Lys-His(3) sequence or sequences, optionally a peptide or chimeric polypeptide is situated carrying at least one epitope from a second species (e.g. human);

alternatively a coiled-coil structed polypeptide is situated after the linker and/or His(6)-Lys-His(3) sequence(s), this coiled-coil structured polypeptide can bind to another coiled-coil structured polypeptide that is linked to an immunogenic moiety, peptide or chimeric polypeptide, and where these coiled-coil polypeptides both are derived from species 1 and therefore are non-immunogenic in species 1,

the first species is a non-human animal, optionally a mammal, optionally a rabbit, goat or llama, or the ferritin polypeptide is derived from a non-human animal, optionally a mammal, optionally a rabbit, goat or llama, and optionally the immunogenic peptide or polypeptide comprises a chimeric immunogenic peptide or polypeptide, and the chimeric immunogenic peptide or polypeptide comprises human immunogenic sequence inserted in a rabbit peptide or polypeptide, and the rabbit polypeptide residues are non-immunogenic when injected into a rabbit; and/or

the non-immunogenic rabbit peptide or polypeptide sequence is derived from a rabbit immunoglobulin polypeptide.

In alternative embodiments of the chimeric or recombinant polypeptide as provided herein:

(a) the ferritin polypeptide comprises at least one first coiled-coil protein or motif that can bind to a second coiled-coil protein or motif (optionally the second coiled-coil protein or motif comprises or is bound to an immunogenic peptide, optionally covalently attached by a non-immunogenic linker), wherein the first coiled-coil protein or motif is attached to the ferritin polypeptide by a non-immunogenic linker, resulting in a chimeric ferritin-coiled-coil protein polypeptide, which optionally can fold into tertiary structure or a helical bundle structure,

and optionally the coiled-coil protein or motif is derived from the first species, and optionally the coiled-coil protein or motif derived from the first species binds to another coiled-coil protein or motif derived from the first species,

and optionally the ferritin polypeptide comprises two, three, four or more first coiled-coil proteins or motifs,

and optionally the coiled coil protein or motif comprises a gamma-aminobutyric acid type B receptor subunit 1 isoform X1 (GBR1) and/or gamma-aminobutyric acid type B receptor subunit 2 (GBR2)), wherein the GBR1 can selectively bind to GBR2 motif,

and optionally the GBR1 motif comprises:

(SEQ ID NO: 33)
STNNNEEEKSRLLEKENRELEKIIAEKEERVSELRHQLQSR,

and optionally the GBR2 motif comprises:

(SEQ ID NO: 34)
SVNQASTSRLEGLQSENHRLRMKITELDKDLEEVTMQLQDT;

(b) the ferritin polypeptide has inserted into its amino acid sequence at least one His(6)-Lys-His(3) (SEQ ID NO:32) moiety, or a plurality of His(6)-Lys-His(3) (SEQ ID NO:32) moieties;

(c) the substantially non-immunogenic linker comprises a poly-G linker or poly-(GGGGS) linker (SEQ ID NO:31);

(d) the poly-(GGGGS) linker (SEQ ID NO:31) comprises or consists of a (GGGGS)₅ (SEQ ID NO:29) linker;

(e) the non-immunogenic linker is attached to the amino terminus of the ferritin polypeptide;

(f) the first species is a rabbit, or the ferritin polypeptide is derived from a rabbit;

(g) the immunogenic peptide or polypeptide comprises a chimeric immunogenic peptide or polypeptide, and the chimeric immunogenic peptide or polypeptide comprises human immunogenic sequence inserted in a rabbit peptide or polypeptide, and the rabbit polypeptide residues are non-immunogenic when injected into a rabbit; and/or

(h) the non-immunogenic rabbit peptide or polypeptide sequence is derived from a rabbit immunoglobulin polypeptide.

In alternative embodiments, provided are products of manufacture comprising a plurality of chimeric or recombinant polypeptides as provided herein,

and optionally the product of manufacture comprises 24 of the chimeric or recombinant polypeptides,

and optionally each of the chimeric or recombinant polypeptides comprises a coiled-coil protein, and the coiled-coil proteins bind to each other.

In alternative embodiment, provided are methods for generating an epitope-specific antibody response in a rabbit, wherein the immune response comprises generation of rabbit antibodies specifically against (or that specifically bind to) at least one human epitope, and the method comprises administering to a rabbit a sufficient amount of a chimeric or recombinant polypeptide as provided herein, to generate the epitope-specific antibody response.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A-C illustrates the transfer of human epitopes onto the rabbit scaffold:

FIG. 1A shows a sequence alignment of human (SEQ ID NO:2) and rabbit (SEQ ID NO:1) λLC constant domain sequences, where sequence differences were found using the sequence alignment;

FIG. 1B schematically illustrates epitopes with species specific sequence that were situated in the selected hidden region which was selected; and,

FIG. 1C shows chimeric sequences: rhLAC1 (SEQ ID NO:3); rhLAC2+3

(SEQ ID NO:4); and, rhLAC7 (SEQ ID NO:5); with the selected epitopes (black, underlined and in bold) grafted onto a rabbit backbone sequence (teal-colored), these sequences were synthesized and inserted into expression vectors,

as discussed in detail in Example 1, below.

FIG. 2A-C illustrates images showing the expression of chimeric proteins, where expression constructs were transformed into cells from a relevant organism and used for production of chimeric protein:

FIG. 2A illustrates SDS-PAGE page images, where the protein was purified using standard methods such as HIS-trap columns and size exclusion columns, and protein expression was verified by SDS-PAGE demonstrating overexpression of protein with the expected band at approximately 13-14 kDa:

FIG. 2B illustrates SDS-PAGE images showing the purity after TEV cleavage;

FIG. 2C illustrates Western blotting images showing the purity after TEV cleavage; where in the Western blot a positive control containing E. coli impurities was included (lane labeled 1) to demonstrate functionality of the E. coli anti-pAb used for verification of sample purity.

FIG. 3A-D graphically illustrates data showing that human epitopes inserted onto the rabbit backbone can be specifically recognized by an antibody raised against native human lambda free light chain (hλ-FLC, variable and constant domain): ELISA plates were coated with chimeric λ-LC constant domain (rh-λ-LC-CD) rhLac1 (FIG. 3A), rhLac2+3 (FIG. 3B) and rhLac7 (FIG. 3C) or with rabbit λ-LC constant domain rLac (FIG. 3D). DAKO A0101 (rabbit polyclonal anti-human λ-FLC antibody, red line) was used as the primary antibody followed by secondary HRP conjugated goat polyclonal anti-rabbit IgG antibody reagent (P0448) in a standard procedure with TMB as the color agent.

FIG. 4A-E graphically illustrates data showing that polyclonal antibody (pAb) derived by immunization with chimeric lambda light chain constant domain (λ-LC-CD) is specific for human λ-LC; purified chimeric proteins carrying human epitopes on a rabbit λ-LC-CD scaffold were used for immunization of rabbits; anti-serum was collected, and the Ig fraction purified; the pAb (blue line) was used as primary antibody in a standard ELISA assay as described in FIG. 3: FIG. 4A-C shows that wells coated with 1 μg/mL chimeric λ-LC-CD rhLAC1 (FIG. 4A), rhLAC2+3 (FIG. 4B) and rhLAC7 (FIG. 4C) were recognized by this primary antibody indicating that chimeric proteins direct the rabbit's immune system to elicit pAb against the selected epitopes in the human λ-LC protein; this was further supported by the fact that wells coated with rabbit λ-LC (FIG. 4D) were not recognized by pAb; and wells coated with 1 μg/mL human λ-LC (variable and constant domain) (FIG. 4E) were also recognized by this pAb and shows that chimeric λ-LC-CD can elicit pAb against native human λ-LC.

FIG. 5A-B schematically illustrates a human lgG molecule, showing that the lgG molecule consists of two heavy chains (gray) and two light chains (colored). There are two variants of light chains: kappa and lambda. Both contains a variable domain (blue) and a constant domain (red). The interaction between heavy and light chain in whole (intact lgG with both heavy and light chain paired together) shields a part of the light chain. The shielded light chain part is denoted the “hidden surface” and the rest the “exposed surface”.

FIG. 6 illustrates the sequence alignment of human (SEQ ID NO:6) and rabbit (SEQ ID NO:7) lambda light chain constant domain and 3D structure; the domain has a beta sandwich made up of two pairing beta sheets: the red sheet consists of four beta strands, and the blue of three, and the red beta strands constitute the hidden surface.

FIG. 7 schematically illustrates an exemplary process for preparing an immunogen (or antigen) as provided herein for generating an antibody or polyclonal antibodies, wherein the immunogen generates a polyclonal antibody against an epitope or epitopes of one species inserted in a protein, optionally a homologous protein, from a second species, where the polyclonal antibody is made in the second species.

FIG. 8 schematically illustrates an exemplary process for preparing an immunogen (or antigen) as provided herein for generating an antibody or polyclonal antibodies, wherein the immunogen is engineered or designed to lack one or more epitopes such that when the immunogen is used to generate polyclonal antibodies, no antibodies are generated against the removed epitope or epitopes.

FIG. 9 schematically illustrates an exemplary process for preparing an immunogen (or antigen) as provided herein for generating an antibody or polyclonal antibodies, wherein the immunogen is engineered or designed to comprise an additional epitope or epitopes such that when the immunogen is used to generate polyclonal antibodies, antibodies specific for the additional epitope or epitopes are generated.

FIG. 10 schematically illustrates an exemplary process for preparing an immunogen (or antigen) as provided herein for generating an antibody or polyclonal antibodies, wherein the immunogen is engineered or designed to comprise an modified epitope or epitopes which in unmodified form would not generate an immune response in a second species, but in modified form do generate an immune response in the second species.

FIG. 11 schematically illustrates an exemplary process for preparing an immunogen (or antigen) as provided herein for generating an antibody or polyclonal antibodies, wherein the immunogen is engineered or designed to comprise an modified epitope or epitopes, wherein the epitope or epitopes are modified to be less immunogenic, such that when the immunogen is used to generate polyclonal antibodies a less robust immune response is generated, or the generated polyclonal antibodies bind to a protein with the modified epitope or epitopes less strongly or slower.

FIG. 12 schematically illustrates an exemplary process for preparing an immunogen (or antigen) as provided herein for generating an antibody or polyclonal antibodies.

FIG. 13 illustrates the transfer of human epitopes onto rabbit scaffold, in particular, this figure illustrates the chimeric λ-LC-CD (SEQ ID NO:8) showing the selected epitopes (black, or in bold) (see also FIG. 1A, which illustrates a sequence alignment of human (SEQ ID NO:2) and rabbit (SEQ ID NO:1) λ-LC constant domain sequences, where sequence differences were found using the sequence alignment).

FIG. 14A-B illustrates images of λ-LC-CD with the left-hand image showing human epitopes (yellow) grafted onto the rabbit backbone (deep teal) sequence, as further discussed in Example 2, below.

FIG. 15 illustrates SDS-PAGE images showing the purity after TEV cleavage, as further discussed in Example 2, below

FIG. 16A-B illustrates SDS-PAGE and Western blotting images showing the purity after TEV and SEC purification, as further discussed in Example 2, below.

FIG. 17A-D graphically illustrates data showing that polyclonal antibody (pAb) derived by immunization with refined chimeric lambda free light chain (λ-LC) constant domain is specific for human λ-LC hidden surface; purified chimeric protein carrying human epitopes on a rabbit λ-LC-CD scaffold were used for immunization of rabbits; anti-serum was collected, and the Ig fraction purified (IgGfₑₓₐₘₚₗₑ₂); pAbs (IgGfₑₓₐₘₚᵢₑ₁, blue; A0101, red and IgGfₑₓₐₘₚₗₑ₂, yellow lines) were used as primary antibody in a standard ELISA assay as described in FIG. 3 or in an agglutination assay:

FIG. 17A shows that wells coated with 1 μg/mL (6.67 μM) SEC purified human IgG were recognized by IgGfₑₓₐₘₚₗₑ₁ and A0101 whereas IgGfₑₓₐₘₚₗₑ₂ have significantly less reactivity against intact human IgG indicating that IgGfₑₓₐₘₚₗₑ₁ and A0101 contains paratopes specific for the exposed surface;

FIG. 17B shows that only IgGfₑₓₐₘₚₗₑ₁ contains the property to agglutinate in the presences of intact human IgG;

FIG. 17C positive control shows that all pAb agglutinate in presences of human λ-FLC; and

FIG. 17D Negative control shows that IgGfₑₓₐₘₚₗₑ₁ and IgGfₑₓₐₘₚₗₑ₂ are unable to agglutinate in the presence of rabbit IgG,

thus, supporting ELISA data from FIG. 4D, and indicate that chimeric λ-LC-CD can elicit pAb against native human λ-FLC. FIG. 18A-B illustrate the transfer of human epitopes onto a rabbit scaffold:

FIG. 18A illustrates human—and rabbit Serum Amyloid A (SAA) sequences differences that were found using sequence alignment, with the human sequence as SEQ ID NO:9, and the rabbit sequence as SEQ ID NO:10; and

FIG. 18B illustrates the Chimeric Serum Amyloid A (SAA) sequence (SEQ ID NO:11) showing the selected epitopes (black, underlined and in bold) with species specific sequence that were situated in the hydrophilic region grafted onto a rabbit backbone (red) sequence; and,

FIG. 19A-B illustrates images of SAA with the left-hand image showing human epitopes (yellow) grafted onto the rabbit backbone (red) sequence,

as further discussed in Example 3, below.

FIG. 20A-B illustrate the transfer of human epitopes onto a rabbit scaffold:

FIG. 20A shows how human (SEQ ID NO:12) and rabbit (SEQ ID NO:13) Kappa light chain constant domain (κ-LC-CD) sequence differences were found using sequence alignment, as further discussed in Example 4, below; and

FIG. 20B illustrates a chimeric sequence (SEQ ID NO:14) with the selected Kappa light chain (κ-LC) epitopes (black, underlined, and in bold) grafted onto the rabbit backbone (blue) sequence.

FIG. 21A-B illustrate two images of the Kappa light chain (κ-LC), with FIG. 21A illustrating in yellow (or lighter) color the constant domain (CD) epitopes which were selected and grafted onto a rabbit backbone sequence (the blue, or darker, color in FIG. 21A), these CD epitopes were species specific sequence that are situated in selected hidden regions, and FIG. 21B illustrates an all rabbit backbone sequence, as further discussed in Example 4, below.

FIG. 22A illustrates an SDS-PAGE showing protein expression to demonstrate overexpression of Kappa light chain constant domain (κ-LC-CD) protein with the expected band at approximately 13 kDa to 14 kDa (arrows) in both pellet (P) and supernatant (S), as further discussed in Example 4, below.

FIG. 22B illustrates SDS-PAGE showing chimeric (κ-LC-CD) protein purity after TEV cleavage and SEC, as further discussed in Example 4, below.

FIG. 22C illustrates a Western blot (WB) showing chimeric κ-LC-CD protein purity after TEV cleavage and SEC, as further discussed in Example 4, below.

FIG. 23A Wells were coated with 1 μg/mL (6.67 nM) SEC purified human IgG. While positive control Q0499 (light blue) strongly recognized intact human IgG then the negative control A0100 (red) showed poor binding and antiserum against chimeric (κ-LC-CD, yellow) was nearly devoid of reactivity. This demonstrates that the chimeric antigen generates very little, if any, side-reactivity to intact human IgG.

FIG. 23B: Wells were coated with 1 μg/mL (40 nM) native human κLC (variable and constant domains). The two negative controls A0499 (light blue) and A0101 (red/grey) did not react with human κ-LC whereas both the positive control, A0100 (red/orange), and antiserum (yellow) from rabbits immunized with chimeric κ-LC-CD gave strong signals. This demonstrates that the chimeric constant domain directs an immune response towards epitopes present in native human κFLC.

FIG. 23C: Wells were coated with 1 μg/mL (80 nM) chimeric κ-LC-CD. The anti-rabbit IgG antibody-HRP visualization reagent gave high background but whereas no signal could be detected when using the negative controls Q0499 (light blue) and A0101 (red/grey) then signal above background could be seen with both the positive control A0100 (red/orange) and to an even higher degree with the antiserum from rabbits immunized with chimeric κ-LC-CD. This demonstrates that both the antiserum and A0100 recognized the chimeric κ-LC-CD.

FIG. 23D: Wells were coated with 1 μg/mL (80 nM) recombinant rabbit kappa LC constant domain r-κ-LC-CD. Any binding of antiserum to κ-LC-CD was below background. This stands in contrast to the high level of binding to the chimeric constant domain (FIG. 23C), indicating that the polyclonal antibody in the antiserum is specific for the human epitopes inserted into the rabbit scaffold.

FIG. 24A Agglutination experiments with 1 mg/mL (6.67 μM) SEC purified human IgG. While positive control Q0499 (light blue) strongly agglutinate intact human IgG then the negative control A0100 (red) and antiserum against chimeric κ-LC-CD yellow) are not able to agglutinate. Together with ELISA data from FIG. 23A, this indicates that the chimeric antigen generates very little, if any, side-reactivity to intact human IgG.

FIG. 24B Agglutination experiments with 1 mg/mL (6.67 μM) SEC purified rabbit IgG. No agglutination is observed with A0499 (light blue), A0100 (red/orange) or antiserum (yellow) from rabbits immunized with chimeric κ-LC-CD. This demonstrates that the chimeric constant domain does not directs an immune response towards self (rabbit) sequence.

FIG. 24C Agglutination experiments with 1 mg/mL (40 μM) native human κFLC. Agglutination is observed with A0100 and with antiserum from rabbit immunized with chimeric κ-LC-CD. This supports that the inserted human epitopes direct an immune response towards the native human antigen.

FIG. 24D Agglutination experiments with 1 mg/mL (80 μM) chimeric κ-LC-CD. Antiserum (yellow) and A0100 (red) both agglutinate with chimeric κ-LC-CD showing that the applied epitopes can be bound by more than one antibody. By extension this indicates that at least two antibodies can bind simultaneously to the hidden surface.

FIG. 25A-D illustrate how human—and rabbit gamma immunoglobulin (IgG) sequences differences were found using sequence alignment; chimeric sequences with the selected epitopes (FIG. 25A: colored and underlined; FIG. 25B, underlined and bolded) were grafted onto the rabbit backbone sequence, and were synthesized and inserted into expression vectors; rabbit backbone (SEQ ID NO:15); rhIgG1 (SEQ ID NO:16); rhIgG2 (SEQ ID NO:17); rhIgG2_2 (SEQ ID NO:18); rhIgG3 (SEQ ID NO:19); rhIgG4 (SEQ ID NO:20), as further discussed in Example 5, below.

FIG. 26A-F illustrate Rabbit and chimeric IgG made by methods as provided herein, where human isotypic specific epitopes (colored, or darker than the grey IgG backbone) were grafted onto rabbit IgG backbone (grey); where FIG. 26A illustrates a rabbit IgG backbone without human epitopes inserted; and FIG. 26B illustrates a chimeric IgG1 subtype with human epitopes inserted (colored, or darker), FIG. 26C illustrates a chimeric IgG2 subtype with human epitopes inserted (colored, or darker), FIG. 26D illustrates a chimeric IgG2_2 subtype with human epitopes inserted (colored, or darker), FIG. 26E illustrates a chimeric IgG3 subtype with human epitopes inserted (colored, or darker), and FIG. 26F illustrates a chimeric IgG4 subtype with human epitopes inserted (colored, or darker), as further discussed in Example 4, below.

FIG. 27A-F illustrate data showing how human epitopes can be substituted onto a non-human polypeptide background to generate an anti-human epitope specific response:

FIG. 27A illustrates constructed rabbit IgG carrying (or having inserted therein) epitopes specific for human IgG1, IgG2 IgG3 and IgG4;

FIG. 27B-E graphically illustrate data showing the rabbit immune response of human IgG1, IgG2 IgG3 and IgG4 epitopes in rabbit Ig, respectively;

FIG. 27F illustrates re-designed IgG1 or IgG2 subtypes to improve the rabbit's reactivity to the Igl and IgG2 epitopes, SEQ ID NO:21 is the rabbit backbone, SEQ ID NO:22 is rhlgG1_2, and SEQ ID NO:23 is rhlgG2_3,

as discussed in detail in Example 6, below.

FIG. 28A-F illustrate construction of a chimeric antibody with desired properties:

FIG. 28A illustrates a rabbit Serum Amyloid A (SAA) backbone, with red residues (or darker color) being the rabbit SAA sequence, and yellow representing inserted human specific amino acids;

FIG. 28B illustrates a human SAA with blue (or darker) color indicating six hydrophobic residues that may interact with a lipid surface;

FIG. 28C illustrates antibody derived from immunization with the SAA illustrated in FIG. 28A coupled to beads;

FIG. 28D illustrates antibody derived from immunization with the SAA as illustrated in FIG. 28B leads to immune particles having antibodies (Abs) comprising some paratopes that recognizes the human hydrophobic (blue) epitopes;

FIG. 28E-F are diagrams showing the kinetics of C and D reacting to five different levels of SAA,

as discussed in detail in Example 7, below. FIG. 29 graphically illustrates data showing that immunization with an incomplete human epitope can provide for a slow reacting polyclonal antibody (srpAb) to human C-Reactive Protein (CRP), as discussed in detail in Example 8, below.

FIG. 30A-B illustrates an exemplary chimeric ferritin construct (FIG. 30A) comprising an attached immunoglobulin antigen CDv6, where the CDv6 is separately depicted in FIG. 30B, as discussed in detail in Example 9, below.

FIG. 31 illustrates an image of a Western blot showing that pAb used as primary IgG (sample 1108) elicited against immunogen CdV6, the chimeric rabbit human free light chain domain, interacts with the B9 (columns #2 and #1 are different purification fractions) and a “20 fraction” from a size exclusion, as discussed in detail in Example 9, below.

FIG. 32 graphically illustrates data showing dynamic light scattering (DLS), or size distribution by intensity (size being a function of intensity), as discussed in detail in Example 9, below.

FIG. 33 graphically illustrates data showing that CDv6 expressed fused to Ferritin is correctly folded, as discussed in detail in Example 9, below.

FIG. 34A illustrates the sequence of an exemplary recombinant ferritin core presenting (or comprising) a modified CdV6 having human epitopes inserted therein (SEQ ID NO:35),

and the subsequences are:

Chimeric CdV6
(SEQ ID NO: 28)
GQPAVTPTVTLFPPSSEELKDNKATLVCLISDFYPGAVTVNWKADGNSVTQ
GVETTKPSKQSNNKYAASSYLSLSANQWKSYQSVTCQVTHEGHTVEKSLAP
TECS
Linker
(SEQ ID NO: 29)
GGGGSGGGGSGGGGSGGGGSGGGGS
rabbit ferritin
(SEQ ID NO: 30)
MTSQIRQNYSPEVEAAVNHLVNLHLRASYTYLSLGFYFDRDDVALEGVSHF
FRELAEEKREAAERLLKMQNQRGGRALFQDVQKPSQDEWGKTLNAMEAALA
LEKNLNQALLDLHALGSAHTDPHLCDFLENHFLDEEVKLLKKMGDHLTNIR
RLSGPQASLGEYLFERLTLKHD 175,

as discussed in detail in Example 9, below.

FIG. 34B (SEQ ID NO:36) illustrates a heterodimer formed by the non-covalent binding of: (1) a chimeric recombinant antigen, where chimeric recombinant antigen is covalently bound to the amino terminal of a coiled coil GBR2 motif (SEQ ID NO:34); to (2) a GBR1 motif (underlined) (SEQ ID NO:33) is bound to the amino terminal of a ferritin molecule by use of a non-immunogenic linker (bolded) (SEQ ID NO:29); where the two subunits of the heterodimer are non-covalently bound by the associate of the GBR1 motif to GBR2 motif:

(SEQ ID NO: 36)
STNNNEEEKSRLLEKENRELEKIIAEKEERVSELRHQLQSRGGGGSGGGGS
GGGGSGGGGSGGGGSMTSQIRQNYSPEVEAAVNHLVNLHLRASYTYLSLGF
YFDRDDVALEGVSHFFRELAEEKREAAERLLKMQNQRGGRALFQDVQKPSQ
DEWGKTLNAMEAALALEKNLNQALLDLHALGSAHTDPHLCDFLENHFLMDE
EVKLLKKMGDHLTNIRRLSGPQASLGEYLFERLTLKHD-C-terminal

Like reference symbols in the various drawings indicate like elements.

### DETAILED DESCRIPTION

In alternative embodiments, provided are chimeric immunogens, and methods for making and using them, including methods for obtaining polyclonal antibodies specific for selected epitopes.

In alternative embodiments, provided are methods comprising immunizing an animal with a chimeric or recombinant immunogen as provided herein, for example, the animal is immunized with a modified version of one of its naturally occurring proteins, or a part thereof, that carries selected epitopes derived from a protein, for example, a homologous protein, from another type of animal or species. This artificial hybrid protein or protein domain, i.e., the chimeric or recombinant immunogen as provided herein, causes the animal to produce polyclonal antibodies specific for the selected epitope or epitopes derived from the protein from another type of animal or species.

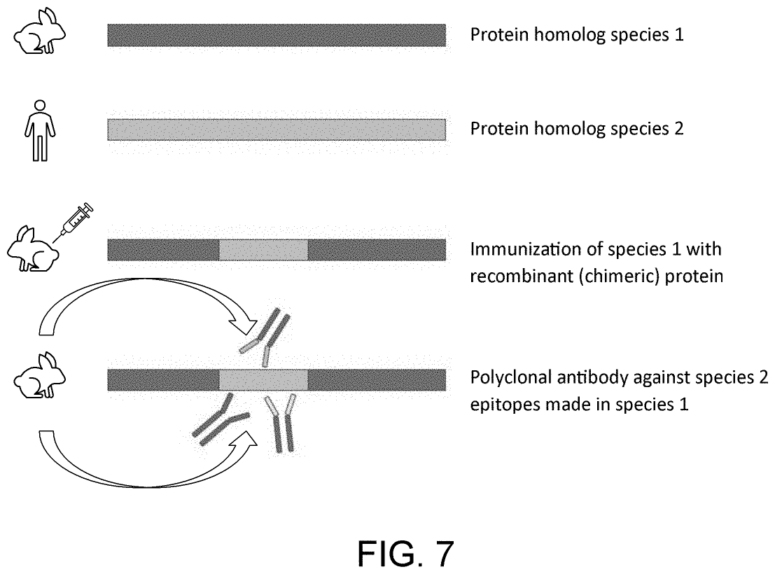

In alternative embodiments, provided are exemplary processes for preparing an immunogen (or antigen) as provided herein for generating an antibody or polyclonal antibodies, wherein the immunogen generates a polyclonal antibody against an epitope or epitopes of one species inserted in a protein, optionally a homologous protein, from a second species, where the polyclonal antibody is made in the second species, as illustrated in FIG. 7.

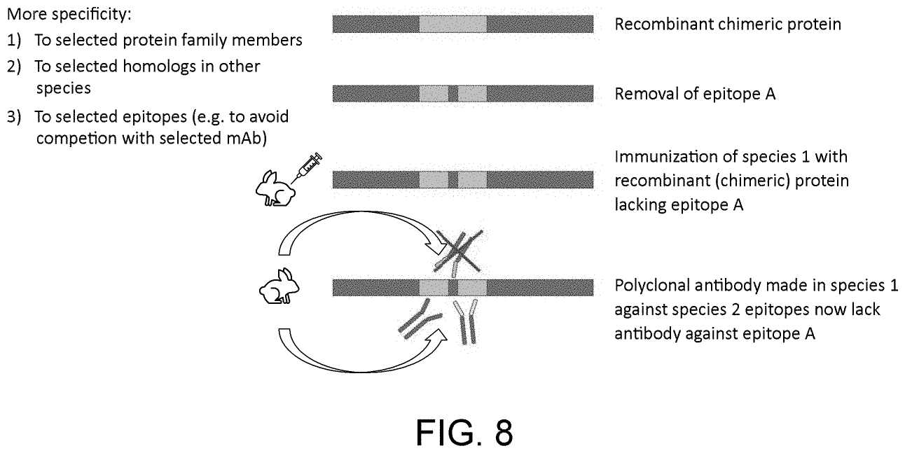

In alternative embodiments, provided are exemplary processes for preparing an immunogen (or antigen) as provided herein for generating an antibody or polyclonal antibodies, wherein the immunogen is engineered or designed to lack one or more epitopes such that when the immunogen is used to generate polyclonal antibodies, no antibodies are generated against the removed epitope or epitopes, as illustrated in FIG. 8.

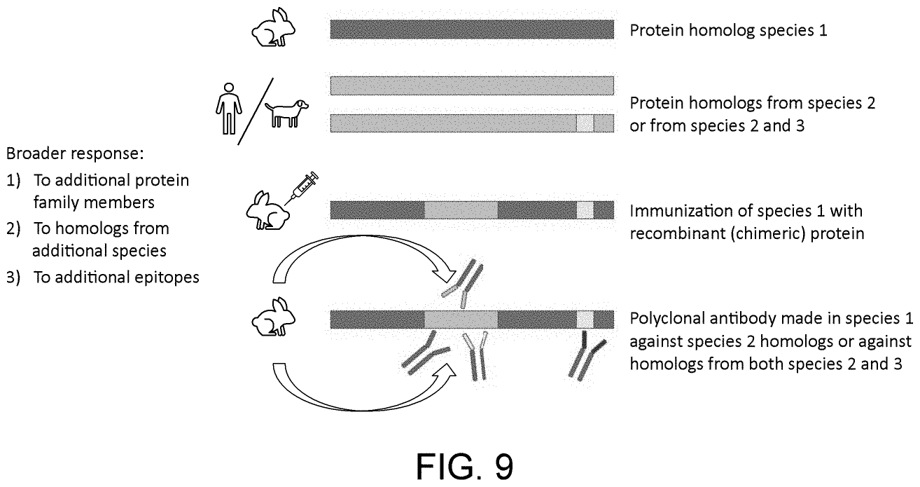

In alternative embodiments, provided are exemplary processes for preparing an immunogen (or antigen) as provided herein for generating an antibody or polyclonal antibodies, wherein the immunogen is engineered or designed to comprise an additional epitope or epitopes such that when the immunogen is used to generate polyclonal antibodies, antibodies specific for the additional epitope or epitopes are generated, as illustrated in FIG. 9.

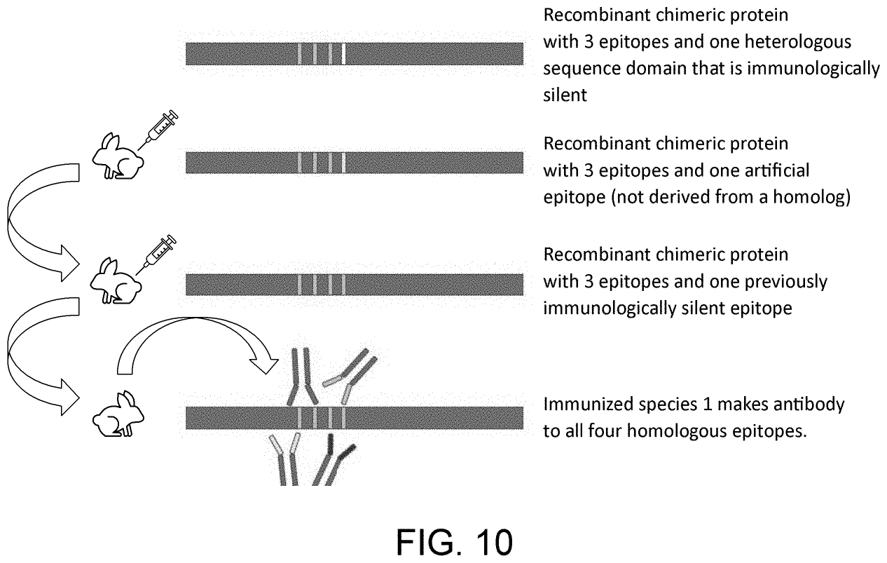

In alternative embodiments, provided are exemplary processes for preparing an immunogen (or antigen) as provided herein for generating an antibody or polyclonal antibodies, wherein the immunogen is engineered or designed to comprise an modified epitope or epitopes which in unmodified form would not generate an immune response in a second species, but in in modified form do generate an immune response in the second species, as illustrated in FIG. 10.

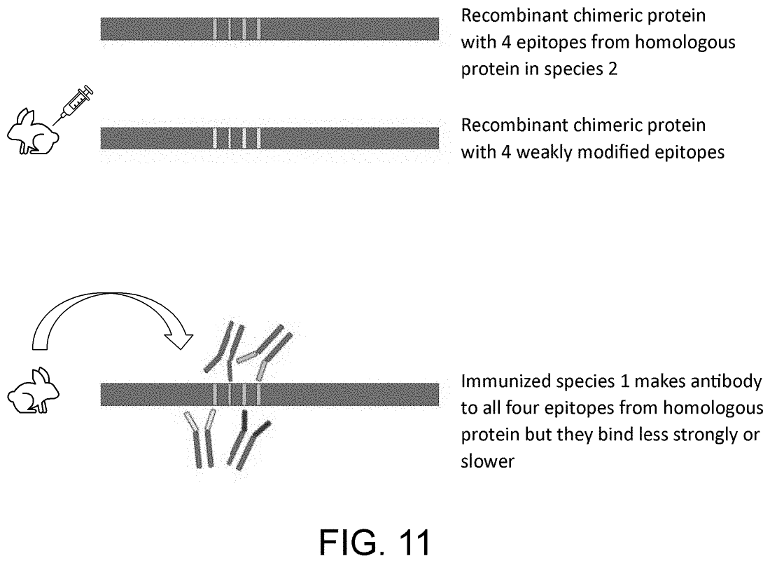

In alternative embodiments, provided are exemplary processes for preparing an immunogen (or antigen) as provided herein for generating an antibody or polyclonal antibodies, wherein the immunogen is engineered or designed to comprise an modified epitope or epitopes, wherein the epitope or epitopes are modified to be less immunogenic, such that when the immunogen is used to generate polyclonal antibodies a less robust immune response is generated, or the generated polyclonal antibodies bind to a protein with the modified epitope or epitopes less strongly or slower, as illustrated in FIG. 11.

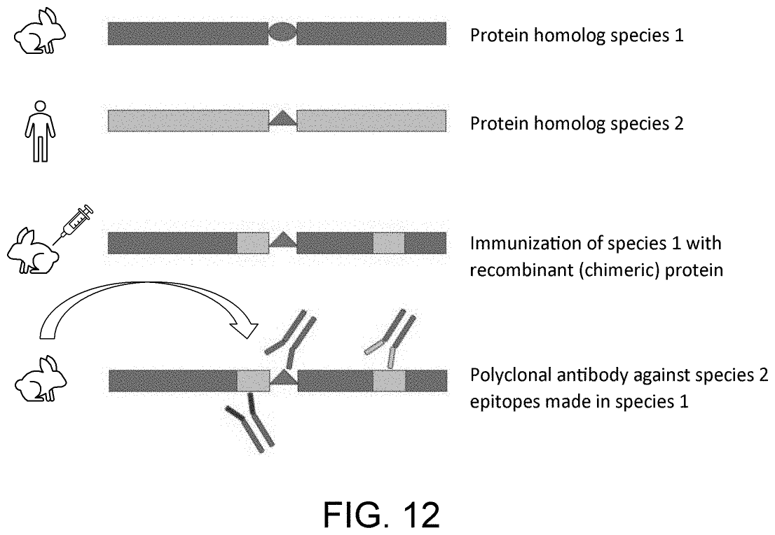

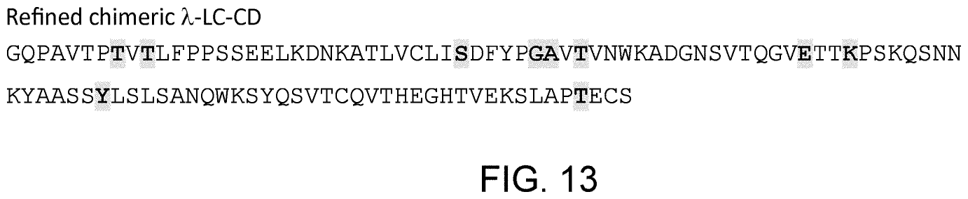

In alternative embodiments, provided are exemplary processes for preparing an immunogen (or antigen) as provided herein for generating an antibody or polyclonal antibodies, as illustrated in FIG. 12.

### Chimeric or Recombinant Polypeptides and Nucleic Acids

In alternative embodiments, provided are chimeric or recombinant polypeptides and methods for making and using them. In alternative embodiments, provided are chimeric or recombinant nucleic acids encoding and expressing polypeptides as provided herein, including expression vehicles containing and expressing these nucleic acids, and cells for containing and expressing these nucleic acids, and also including whole organism expression systems.

In alternative embodiments, recombinant polypeptides as provided herein can be prepared and expressed performed using any method known in the art, including for example using whole organisms such as fungi, plants or animals such as mice, as well as cell cultures derived from whole organisms (such as mammalian cells in culture), or using single cell organisms such as algae, fungal, yeast, insect (for example, baculovirus) or bacterial cells.

The choice of organism to make (for example, recombinantly generate) a chimeric or recombinant polypeptide and/or nucleic acid as provided herein can depend on several factors, including whether secondary modification such as glycosylation is desired or required, or whether the protein is desired or required to be associated with or inserted in a membrane system (for example, in situ), or if a particular protein folding pattern is desired or required, and/or is a di-sulfide bridge formation is desired or required.

In alternative embodiments, a nucleic acid for expressing a chimeric or recombinant polypeptide as provided herein, for example for expression in vitro or in vivo, is contained in an expression vehicle, for example, in an expression cassette, vector, recombinant virus, artificial chromosome, a cosmid or a plasmid. In alternative embodiments, the nucleic acid or expression vehicle expressing a chimeric or recombinant polypeptide as provided herein is administered to an animal (for example, as naked DNA, which can be appropriately formulated) for the purpose of that animal generating a humoral immune response against an epitope in the recombinant polypeptide as provided herein.

In alternative embodiments, a protein-coding DNA sequence, which can be in an expression vehicle, is transferred to the organism or cell and placed under control of relevant expression elements such as a transcriptional promoter, an enhancer and/or a polyadenylation signal sequence. In alternative embodiments, a protein sequence as provided herein is processed in a specific cellular organelle(s), and this may require addition of one or more localization signals such as a periplasm localization sequence.

In alternative embodiments, a protein-coding DNA sequence (for example, as an expression vehicle) is inserted into a genome (stably or not), or can be alternatively episomal. Recombinant protein expression systems can be transient or permanent.

In alternative embodiments, for example to enhance the ability of a given protein to act or function as an antigen or immunogen for immunization purposes, the recombinantly produced protein is purified; for example, the presence of impurities may result in an immunized animal making antibodies against irrelevant targets; and in the presence of too much impurity, formation of high amounts of a desired antibody may be counteracted and removal of reactivity against the impurities from the polyclonal antibody may be time consuming and costly.

In alternative embodiments, purification of a protein species is done based on the specific characteristics of the desired protein, for example, purification comprises using hydrophobicity, charge and/or size using chromatographic means such as hydrophobic interaction chromatography (HIC), ion exchange chromatography (IEC) and/or size exclusion chromatography (SEC). In alternative embodiments, specific protein interactions are used for purification purposes, for example, using affinity purification, or lack of specificity of the protein is used to remove other protein species, for example, using absorption purification. In alternative embodiments, antibodies or other protein-specific binding proteins are used for affinity purification and/or absorption purification the protein.

In alternative embodiments, when expressing a protein recombinantly, protein sequences are added that allow for specific purification methods such as for example, an epitope tags such as FLAG, hemagglutinin (HA), c-myc, T7, Glu-Glu, ALFA-tag, V5-tag, Myc-tag, HA-tag, Spot-tag, T7-tag and NE-tag; a biotin and streptavidin or avidin system; a polyhistidine affinity tag such as a small HIS-tag (6-8 amino acids) (and optionally using immobilized metal affinity chromatography); an N-terminal glutathione S-transferase (GST) molecules followed by protease cleavage sites; a 43 kDa large Maltose Binding Protein (MBP); an intein-chitin binding domain (intein-CBD) tag; or, a calmodulin binding peptide (CBP) purification system utilizing a C-terminal fragment from muscle myosin light-chain kinase in order to purify proteins of interest from bacteria. This increases the available tools for purification purposes and makes it possible to use standard methods for many different proteins.

In some cases, it is desired to remove such purification sequences before performing immunization. This can be achieved by placing a protease site between the purification sequence and the actual protein-encoding sequence, for example, the sequence of a chimeric protein as provided herein. One example is the Tobacco Etch Virus (TEV) protease that upon cleavage of a consensus sequence only leaves an N-terminal Glycine residue.

In alternative embodiments, a recombinant protein as provided herein is made in situ in the immunized animal, for example, by modifying cells in an animal to have novel or changed DNA sequences that can code for expression of the recombinant protein, and express and/or secrete those immunogenic proteins.

### Immunization Procedures

In alternative embodiments, provided are methods for making antibodies, or for generating or stimulating an immune response, in an animal, for example, in a mammal (for example, a rabbit, a murine species such as a mouse or a rat, a sheep, a goat, a pig, a cow or a horse) or in a species of the genus Phasianidae (for example, a chicken) comprising administering a chimeric or recombinant protein as provided herein.

In alternative embodiments, to derive a polyclonal antibody against a protein target, a protein derived from one type of animal (species) is used to give an immune response in another type of animal (species).

In alternative embodiments, a chimeric or recombinant protein as provided herein comprising at least one human epitope is used for stimulation of the immune system, for example, for generating a humoral response, in mouse, rat, rabbit, sheep, goat, pig, cow, horse or chicken, and the derived or generated polyclonal antibody or antibodies can specifically recognize the human protein, and can be used to specifically recognize, tag, bind to and/or isolate the human protein from which the at least one human epitope was derived.

In alternative embodiments, a protein from any species is used to immunize another species to generate a humoral immune system as long as the protein used for immunization carries at least one modification (for example, at least one one amino acid difference) compared to any homologous protein or protein domain in the species that is being immunized.

In alternative embodiments, an adjuvant is also used when administering chimeric or recombinant proteins as provided herein. While the administered chimeric or recombinant protein is the agent directing the immune response to make antibody against specific epitopes expressed by the recombinant protein, an adjuvant mixed with the protein can ensure the immune system is activated; for example, by using a adjuvant the protein is placed in a deposit being released into the body over a longer period. In alternative embodiments, different adjuvants are used, for example, adjuvants based on various principles such as the oil-in-water principle, for example

Freund's Adjuvant is used. In alternative embodiments, protein and adjuvant mixtures are injected into one or more subcutaneous locations. In alternative embodiments, the administration procedure is repeated several times (for example, between about 2 to 10 time) to boost the immune response (the boost phase); and, a high production of polyclonal antibody can be maintained by renewing the immunization at regular but typically longer intervals, for example, additional administrations once every 3 to 16 weeks.

### Selecting Epitopes to be Grafted Onto Backbone a Protein

In alternative embodiments, provided are recombinant polypeptides comprising a portion of a first polypeptide from a first species and at least one portion of a second polypeptide from a second species, wherein the at least one portion of the second polypeptide is a homologue of the first polypeptide, and wherein the homologous portion of the second polypeptide comprises an epitope which is not present in the first polypeptide. In alternative embodiments, the homologous protein or protein domains exist in the two species of interest.

In alternative embodiments, homologous proteins are proteins with a similar 3D structure; when proteins have more than 30% identical protein sequence similarity, they have the same 3-D structure in 90% of cases, and proteins with much less sequence identity may still have similar 3-dimensional structure. In alternative embodiments, the 3-D structural similarity between proteins is assessed using for example, a distance matrix alignment (DALI) and as a rule of thumb a Z-score above 8 indicates homology whereas scores from 2 to 8 represents a grey zone.

In alternative embodiments, the backbone protein, or the first polypeptide from a first species, is derived from the species to be immunized (species one) and the epitope sequences are derived from the species that is to be recognized (species two) by the polyclonal antibody.

In alternative embodiments, the epitope sequence to be inserted or constructed into the “background” protein, or the first polypeptide from a first species, is derived by:

first, the two amino acid sequences are aligned, and differences down to one amino acid residue are highlighted;

at least one of (or a plurality of) such amino acid residue differences is selected; and

the backbone sequence (species one) is modified by changing the selected, or the plurality of selected, amino acids.

In alternative embodiments, after the selected epitope or epitopes have been introduced to the backbone sequence, the derived hybrid (or chimeric) protein is recombinantly expressed, and optionally purified for use in the immunization of species one, and the resulting polyclonal antibodies (or monoclonal antibodies derived from this humoral response) can be applied to recognize the protein in species two.

In alternative embodiments, the hybrid or chimeric protein is considered ready for immunization if it can be maintained for at least one day in solution in a concentration of at least about 50 μg per mL. Further quality control can optionally be performed before immunization via immunological and/or biochemical tests or by spectroscopic examination (for example, circular dichroism) to substantiate that the protein structure is correct.

In alternative embodiments, when the polyclonal antibody is to be used for assays working on intact protein, for example, assays such as ELISA, turbidimetry and CLIA assays, it may be an advantage to include further steps, for example:
the two amino acid sequences are aligned, and differences down to one amino acid are highlighted;
the differences are highlighted on the 3D structure of the protein or domain;
differences residing in surface exposed areas are identified;
at least one of such surface exposed differences is selected; and/or,
the backbone sequence (species one) is modified by changing the selected amino acids to those of species two.

In some cases, the 3D structure of the protein or domain may be unknown and the second and third steps cannot be applied; instead, in alternative embodiments, a series of hybrid proteins with different epitope sequences are examined until the desired antibody is derived.

### Exemplary Applications of Making and Using Chimeric Proteins as Provided Herein

Prevention of Undesired Antibody Reactivity or Characteristics

In alternative embodiments, recombinant polypeptides as provided herein are used for, or methods as provided herein further comprise:

increasing specificity for one homologous protein species out of a family, for example, by avoiding epitopes in the applied backbone sequence from species two that exist in other members of the protein family (in species two) such that the immune reaction will be aimed, or more focused, at the remaining epitopes, that are more unique for the selected protein species;

increasing specificity for one domain out of many in a protein; this can be done by removing epitopes that exist in other domains of the protein family (of species two) such that the immune reaction will be aimed at the remaining epitopes that are more unique for the selected domain;

increasing cooperativity of the polyclonal antibody composition for a given application; one example is to obtain reactivity against a sub-set of epitopes to cause fast and efficient cross-binding in a turbidimetric reaction, and another example is to create a polyclonal antibody that can cooperate with a monoclonal antibody in an assay such as ELISA or CLIA (for example, by removing the epitope recognized by the monoclonal antibody);

preventing undesired characteristics of a polyclonal antibody, for example, by selectively removing epitopes from the species two sequence, such that subtypes of paratopes on the antibody are avoided, for example, where key characteristics such as antibody isoelectric point (pI) and hydrophobicity may be influenced or controlled to give desirable characteristics when interacting with other materials (one example is interaction with plastic surfaces);

obtaining a higher degree of control over the manufacturing process of polyclonal antibody such that it is more standardized from batch to batch; one example is to remove one or more immune dominant epitope from the species two sequence until a more consistent reactivity toward minor epitopes is achieved in the immunized animals; another example is to eliminate or remove from the species two polypeptide the weakest epitopes to avoid the more variable response to such elements; and/or

reduce reactivity (for example, the reaction speed) towards a given protein by removing some epitopes and/or reducing the antibody affinity by using (or inserting into the species two sequence) a modified epitope or epitopes, where this is useful for applications such as wide range turbidimetric assays.

Addition of Desired Reactivity or Characteristics

In alternative embodiments, recombinant polypeptides as provided herein are used for, or methods as provided herein further comprise:

multi-species reactivity such that the same antibody can be used for, for example, diagnostics of both humans and animal species; this antibody can be made by inserting additional epitopes into the species one backbone, or by combining or fusing different recombinant proteins with different epitope characteristics, or with different newly inserted epitopes;

multi-protein reactivity such that all or a selected sub-set of a protein family are recognized by a polyclonal antibody; this can be achieved by adding or inserting epitopes into the species one backbone that are different between the family members or by combining hybrid proteins that have different versions of the selected epitopes in the immunization mixture;

multi-domain reactivity such that all or a selected sub-set of a domain type are recognized by a polyclonal antibody; this can be achieved by adding epitopes into the species one backbone that are different between the domains or by combining hybrid domains that have different versions of the selected epitopes in the immunization mixture;

reactivity towards epitopes that do not give a primary response; by using a series of modified epitopes, it is possible to overcome lack of a primary response towards a given epitope as has been shown for development of vaccine against virus, for example, by Escolano, et al., 2016, Cell 166, 1445-1458; this approach with sequential immunization can also be used for production of polyclonal antibody; and/or

enhancing desired characteristics of a polyclonal antibody, for example, by selectively removing epitopes from the species two sequence such that subtypes of paratopes on the antibody are avoided; key characteristics such as antibody pI and hydrophobicity may be influenced or controlled to give desirable characteristics when interacting with other materials, for example, when interacting with plastic surfaces.

In alternative embodiments, humoral immunity is the immune response involving transformation of B cells into plasma cells that produce and secrete antibodies to a specific antigen.

In alternative embodiments, an epitope, also known as antigenic determinant, is the part of an antigen that is recognized by an antibody.

In alternative embodiments, a paratope, also called an antigen-binding site, is a part of an antibody which recognizes and binds to an antigen.

In alternative embodiments, the isoelectric point (pI) is the pH of a solution at which the net charge of a protein becomes zero; at solution pH that is above the pI, the surface of the protein is predominantly negatively charged, and therefore like-charged molecules will exhibit repulsive forces.

### Vaccines and Vaccination

In alternative embodiments, provided are vaccine formulations comprising chimeric or recombinant polypeptides, nucleic acids encoding them, including DNA- and RNA-protein encoding molecules (for example, protein-encoding mRNA), or nucleic acid expression vehicles as provided herein, and/or cells as provided herein.

In alternative embodiments, vaccine formulations as provided herein comprise or further comprise an adjuvant or an incomplete adjuvant, or a pharmaceutically acceptable excipient, wherein optionally the pharmaceutically acceptable excipient comprises a sterile buffer, saline or water.

In alternative embodiments, chimeric or recombinant polypeptides, nucleic acids (such as protein-encoding RNA) encoding them or nucleic acid expression vehicles as provided herein are formulated in liposomes, for example, as liposome delivery vehicles having a polycationic lipid composition (for example, cationic liposomes) and/or liposomes having a cholesterol backbone conjugated to polyethylene glycol, where exemplary cationic liposome compositions comprise or are manufactured using: N-[1-(2,3-dioleyloxy)propyl]-N,N,N-trimethylammonium chloride (DOTMA) and cholesterol, N-[1-(2,3-dioleoyloxy)propyl]-N,N,N-trimethylammonium chloride (DOTAP) and cholesterol, 1-[2-(oleoyloxy)ethyl]-2-oleyl-3-(2-hydroxyethyl)-imidazolinium chloride (DOTIM) and cholesterol, dimethyldioctadecylammonium bromide (DDAB) and cholesterol, and combinations thereof.

For example, in alternative embodiments the protein-encoding nucleic acid can be a DNA encoding one or more immunogenic peptides or proteins, and the DNA can be carried in an expression vehicle such as a viral vector, for example an adenovirus vector such as an Ad5 or adeno-associated vector (AAV). In alternative embodiments, recombinant adenoviruses as used in vaccines as provided herein can be as described in U.S. patent application no. US 20200399323 A1, which describes for example recombinant adenoviruses including a deletion in or of the E1 region or any deletion that renders the virus replication-defective, for example, the replication-defective virus can include a deletion in one or more of the E1, E3, and/or E4 regions; or, can be as described in U.S. patent application no. US 20190382793 A1, which described how to make recombinant adenoviruses for gene therapy.

In alternative embodiments, the protein-encoding nucleic acid can be an RNA, for example, mRNA, which can be formulated in a lipid formulation or a liposome and injected for example intramuscularly (IM), for example using formulations and methods as described in U.S. patent application no. US 20210046173 Al, which describes delivering to a subject (for example, via intramuscular administration) an immunogenic composition that comprises a RNA (for example, mRNA) that comprises an open reading frame (ORF) that comprises (or consists of, or consists essentially of) an immunogenic or antigenic sequence as provided herein; wherein optionally the RNA (or the DNA-carrying expression vehicle) is formulated in a liposome, or a lipid nanoparticle (LNP), or nanoliposome, that comprises: non-cationic lipids comprise a mixture of cholesterol and DSPC, or a PEG-lipid, or PEG-modified lipid, or LNP, or an ionizable cationic lipid; or a mixture of (13Z,16Z)-N,N-dimethyl-2-nonylhenicosa-12,15-dien-l-amine, cholesterol, DSPC, and PEG-2000 DMG. In alternative embodiments, the PEG-lipid is 1,2-Dimyristoyl-sn-glycerol methoxypolyethylene glycol (PEG-DMG), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA), or, the PEG-lipid is PEG coupled to dimyristoylglycerol (PEG-DMG).

In alternative embodiments, chimeric or recombinant polypeptides, nucleic acids encoding them or nucleic acid expression vehicles as provided herein are formulated with or administered with an adjuvant, which for example can comprise: aluminum hydroxide or mineral oil, a stimulator of immune responses such as lipid A, Bortadella pertussis or Mycobacterium tuberculosis derived proteins; for example,

Freund's Incomplete Adjuvant and Complete Adjuvant (Difco Laboratories, Detroit, Mich.); Merck Adjuvant 65 (Merck and Company, Rahway, N.J.); AS-2 (GlaxoSmithKline, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

In alternative embodiments, chimeric or recombinant polypeptides, nucleic acids encoding them or nucleic acid expression vehicles as provided herein are administered in one or multiple dosage regimens.

In alternative embodiments, chimeric or recombinant polypeptides, nucleic acids encoding them or nucleic acid expression vehicles as provided herein, or vaccines as provided herein, are administered at a dosage of between about 100 μg and about 1 mg; or at a dose comprising between about 50 μg and 500 μg; or between about 1mg and about 10 mg. The vaccine can be administered for example in a single dose, or in two, three, four or five or more doses. In one embodiment, the two doses are administered at a one- or two-week intervals.

In alternative embodiments, chimeric or recombinant polypeptides, nucleic acids encoding them or nucleic acid expression vehicles as provided herein, or vaccines as provided herein, are administered via intradermal, transdermal, intranasal (for example, by intranasal drops or intranasal aerosol delivery), intramuscular, subcutaneous or sublingual routes.

In alternative embodiments, chimeric or recombinant polypeptides, nucleic acids encoding them or nucleic acid expression vehicles as provided herein, or vaccines as provided herein, are administered using a syringe, a pneumatic injector or a jet injection device.

### Products of Manufacture and Kits

Provided are products of manufacture and kits for practicing methods as provided herein, including for example nucleic acids such as expression vehicles for expressing chimeric or recombinant polypeptides as provided herein, or chimeric polypeptides as provided herein, or cells expressing chimeric or recombinant polypeptides as provided herein, or vaccine formulations as provided herein, for example, comprising chimeric or recombinant polypeptides as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby are incorporated by reference. Citation of the above patents, patent applications, publications and documents are not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Making and Using Chimeric Immunogens

This example demonstrates exemplary methods for making and using chimeric immunogens as provided herein.

### Material and Methods

### Epitope Selection

Human epitopes were identified based on sequences alignment and epitope mapping conducted using existing DAKO/Agilent polyclonal antibody A0101 and the constant domain of 1 human free light chain. The identified epitopes were used to design chimeric variants reacting against species 2 and not species 1.

### Protein Expression and Purification

All constructs encoding chimeric variants of λ-FLC were ordered at GENSCRIPT® or cloned into pET22(+). The constructs were designed with an N-terminal His-tag followed by a TEV cleavage site in order to separate the His-tag from the λ-FLC constant domain. Recombinant rabbit λ-FLC constant domain, and chimeric constant domain variants with human epitopes grafted onto the rabbit scaffold, were periplasmic expressed using E. coli strain BL21 (Invitrogen™). The lysogeny broth medium containing recombinant protein was dialyzed against binding buffer (20 mM Na₂HPO₄, 150 mM NaCl, pH 7.4) before recombinant protein were immobilized by affinity chromatography (IMAC) using a His-tag column (GE Healthcare). Immobilized protein was washed with wash buffer (20 mM Na₂HPO₄, 1 M NaCl, 20 mM Imidazole, pH 7.4) with at least 15 column volumes, and eluted with elution buffer (20 mM Na₂HPO₄, 150 mM NaCl, 500 mM Imidazole, pH 7.4). His-tagged recombinant protein was dialyzed into cleavage buffer (20 mM Tris, 150 mM NaCl, pH 8) before adding TEV protease (0.2 mg/mL, final concentration) supplemented with 2 mM reduced L-Glutathione (final concentration). The cleavage reaction was left overnight at +4° C. To separate cleaved recombinant protein from His-tag, TEV protease, and un-cleaved recombinant protein the mixture was loaded onto His-tag column, and flow through was collected and contain the untagged recombinant protein. The sample was further purified using size exclusion chromatography (SEC) SUPERDEX 75™ Prep Grad (GE Healthcare) with binding buffer as eluent. For analysis (ELISA—and turbidimetric assays) recombinant proteins could retain the His₈-tag.

### SDS-PAGE and Western Blotting

Protein purity was followed by SDS-PAGE using pre-caste NUPAGE™ 4-12% Bis-Tris gels (Invitrogen™). All protein samples were loaded with SDS sample buffer (350 mM Tris.HCL, 357 mM Sodium dodecyl sulfate, 44.6% Glycerol, 179 μM Bromophenol blue, pH 6.8) and carried out in a IVIES SDS running buffer) (NOVEX®. Gels were stained with SimplyBlue™ (Invitrogen™). For Western blotting NUPAGE™ 4-12% Bis-Tris gels (Invitrogen™) and MES SDS running buffer (NOVEX™) were used to separate the proteins. The electro blotting was carried out at 30 V for 1 hour, and proteins were transferred to PVDF membrane (BioRad) in Western blot buffer (25 mM Tris, 0.192 M Glycine and ethanol 25.3%). The blotting was followed by a blocking step using blocking buffer (50 mM Tris-HCL, 0.5 M NaCl, 0.5% Tween20, pH 9.0). Blocked PVDF membrane containing transferred protein was incubated for 1 hour (minimum) with primary pAb (rabbit anti-E. coli diluted 1000×) overnight at +4° C. with shaking. Before membrane was incubated for 1 hour with secondary pAb (swine anti-rabbit) the membrane was washed 4×10 min with blocking buffer. After incubation with secondary pAb membrane was washed 4×10 min with blocking buffer and incubated 20 min with DAB (diaminobenzidine) and substrate.

### Antigen Preparation and Immunization

Based on SDS-PAGE and Western blotting analysis of fractions from SEC purification a highly pure antigen sample was produced. Only fractions without impurities were included in the final antigen sample. The antigen sample constitute equal amount of three variants chimeric lambda-FLC (LAC1, LAC2+3, LAC7) to elicit pAb ensemble including paratopes for four isoforms of human free light chains. Antigen sample were mixed in equally amount (1:1) with Freunds incomplete adjuvant (FIA) just before immunizing three-month-old rabbits subcutaneously. Sera were collected before immunization and seven weeks after last immunization. Sera were preserved by adding sodium azide (NaN₃) final concentration 15 mM and stored a 4° C.

### Enzyme-Linked Immunosorbent Assay (ELISA)

All antigens (chimeric variants of λ-FLC-, rabbit λ-FLC constant domain, human λ-FLC or human intact IgG) were diluted to 1 μg/mL with coating buffer (10 mM Na₂HPO₄, 145 mM NaCl, 0.1% Tween-20, pH 7.2) and used to coat 96 well plates overnight at 4° C. All primary pAb (A0101, X0903 and example 1 IgG fraction (IgG_{example 1})) were diluted with 5% skimmed milk in 3-fold series starting from 10 μg/mL. Plates were washed using wash buffer (10 mM Na₂HPO₄, 500 mM NaCl, 0.1% Tween-20, pH 7.2) and incubated 1 hour with primary pAb at room temperature with agitation. Followed by wash with wash buffer, and incubation with secondary pAb (P0448) diluted in 5% skimmed milk to 10 μg/mL for 1 hour with agitation. Finally, plates were washed with washing buffer and developed for 5 minutes after adding TMB (DAKO S1599) 100 μL per well. Reactions were stop by adding 100 μL 0.5 M H2504 to each reaction well. Results were detected by ELISA reader using SOFTMAX™ 6.2.1 with detection wavelengths 450 nm and 650 nm.

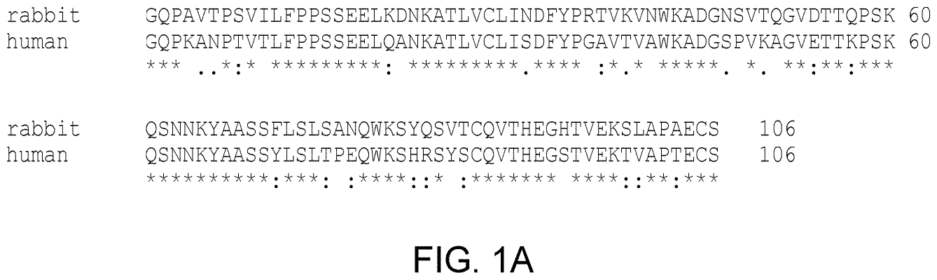

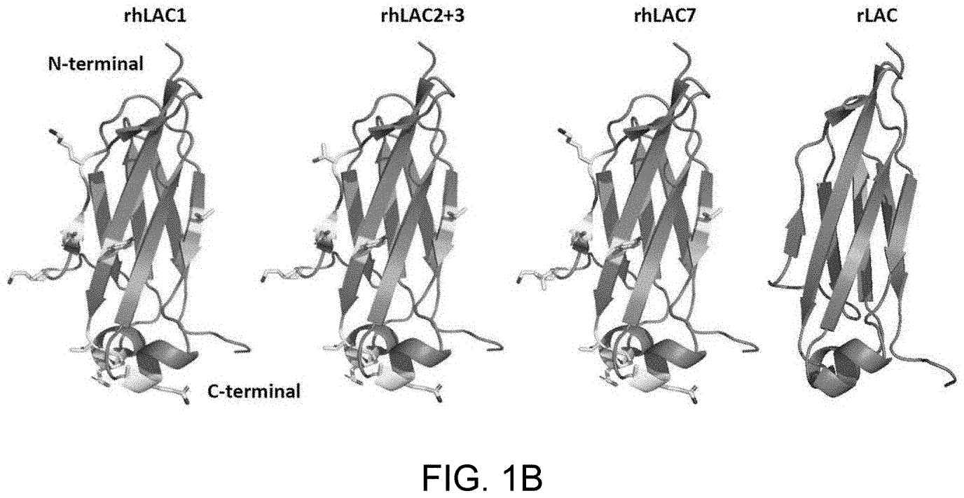

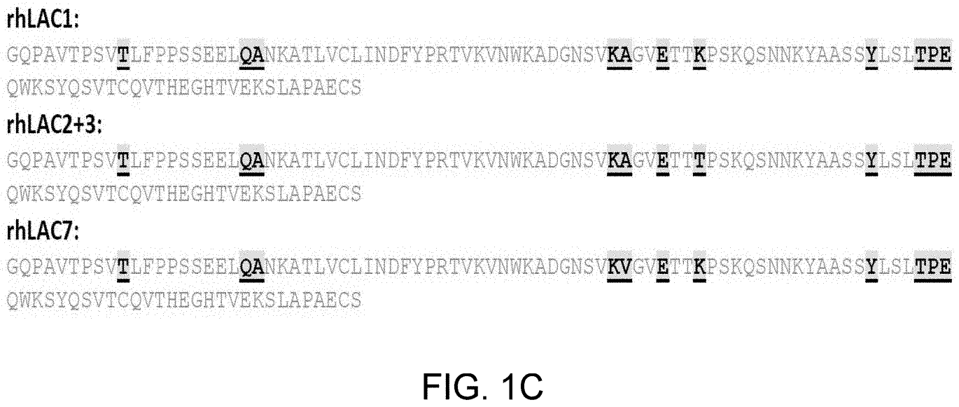

FIG. 1A-C illustrates the transfer of human epitopes onto the rabbit scaffold:

FIG. 1A shows a sequence alignment of human (SEQ ID NO:2) and rabbit (SEQ ID NO:1) λ-FLC constant domain sequences, where sequence differences were found using the sequence alignment;

FIG. 1B schematically illustrates epitopes with species specific sequence that were situated in the selected hidden region which was selected; and,

FIG. 1C shows chimeric sequences: rhLAC1 (SEQ ID NO:3); rhLAC2+3 (SEQ ID NO:4); and, rhLAC7 (SEQ ID NO:5); with the selected epitopes (black, underlined and in bold) grafted onto a rabbit backbone sequence (teal-colored), these sequences were synthesized and inserted into expression vectors.

### Example 2

This example demonstrates exemplary methods for making and using recombinant or chimeric immunogens as provided herein.

This example is similar to Example 1; however, where Example 1 proves the concept, Example 2 uses the concept to develop a specific pAb against λ-FLC. This is achieved by refining selected epitopes.

### Material and Methods

### Epitope Selection

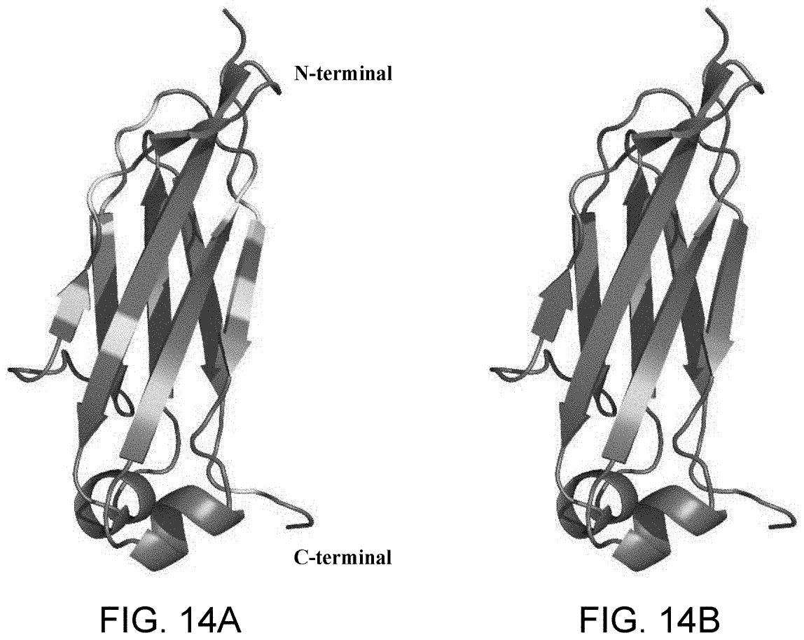

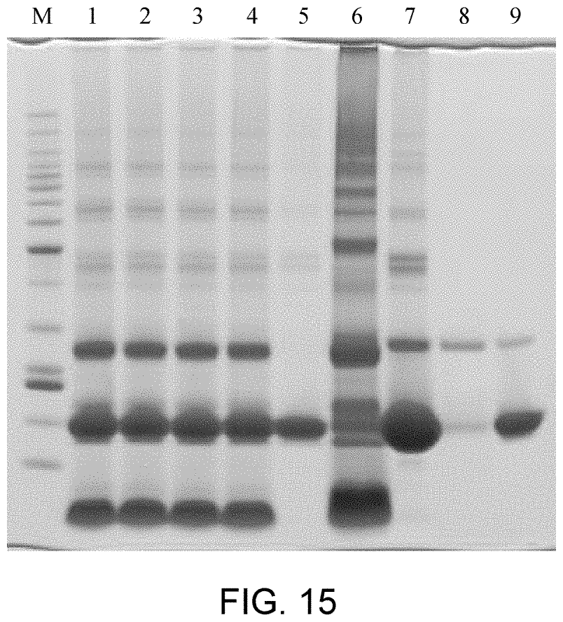

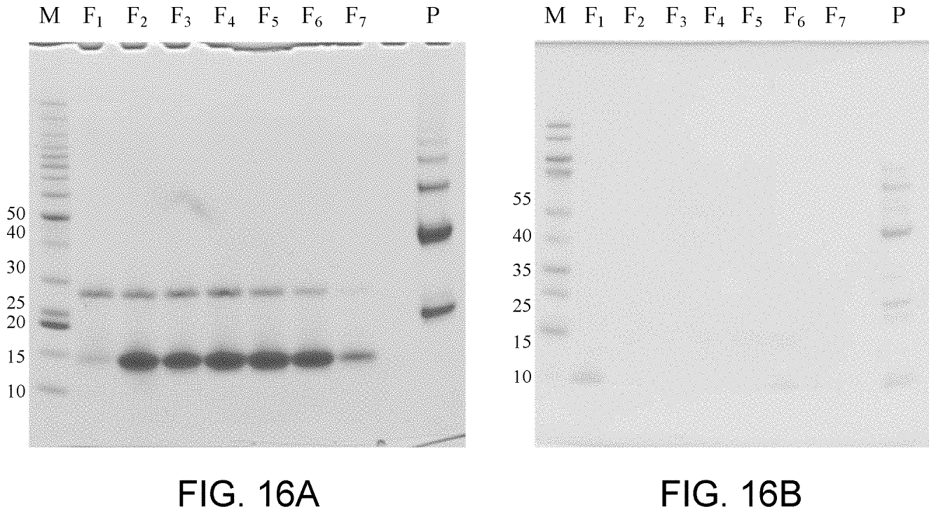

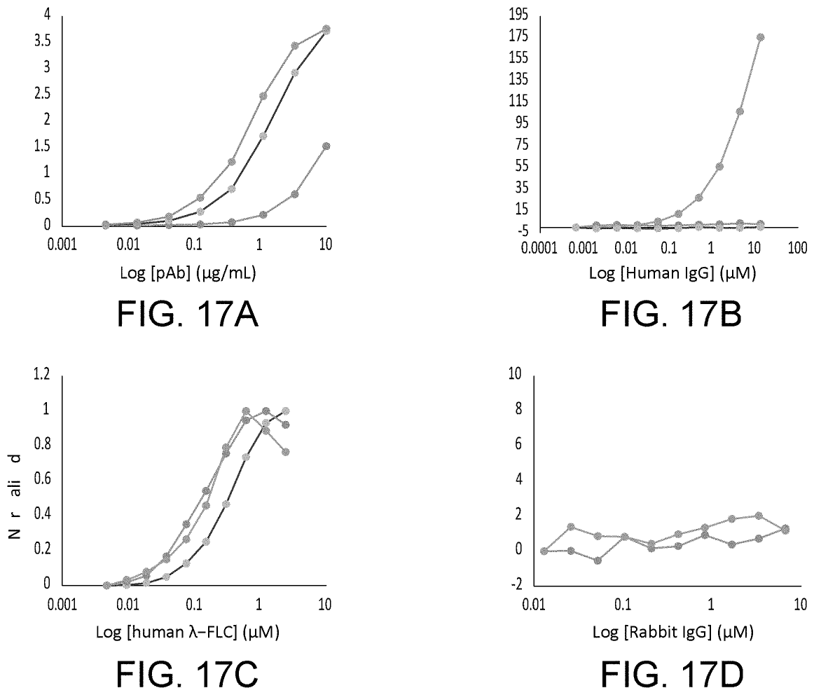

Rabbit and human lambda free light chain (λ-FLC) sequences were fetched from databases (PIR: A30505 and PODOX8, respectively). Sequences were aligned to identify sequence differences in the λ-FLC constant domain between the two species. The selection of epitopes was done by aligning and inspecting the crystal structures of human λ-FLC (PDB entry: 1a8j) and intact human IgG (PDB entry: 1hzh). PISA server was used to identify surface epitopes with less than 10% solvent exposed residues. These residues were included in the chimeric variants to produce a specific λ-FLC pAb, see FIG. 14.

### Protein Expression and Purification

See section in Example 1.

### SDS-PAGE and Western Blotting

See section in Example 1.

### Antigen Preparation and Immunization

Based on SDS-PAGE and Western blotting analysis of fractions from SEC purification a highly pure antigen sample was produced. Only fractions without impurities were included in the final antigen sample. The antigen sample constitute equal amount of three constant domain variants chimeric lambda-FLC (LAC1, LAC2+3, LAC7) to elicit pAb ensemble including paratopes for four isoforms of human free light chains. Antigen sample were mixed in equally amount (1:1) with Freunds incomplete adjuvant (FIA) just before immunizing three-month-old rabbits subcutaneously. Sera were collected before immunization and seven weeks after last immunization. Sera were preserved by adding sodium azide (NaN₃) final concentration 15 mM and stored a 4° C.

### Enzyme-Linked Immunosorbent Assay (ELISA)

Human intact and SEC purified IgG were diluted to 1 μg/mL with coating buffer (10 mM Na₂HPO₄, 145 mM NaCl, 0.1% Tween-20, pH 7.2) and used to coat 96 well plates overnight at 4° C. All primary pAb (A0101, Example 1 IgG fraction (IgGfₑₓₐₘₚₗₑ₁) and Example 2 IgG fraction (IgGfₑₓₐₘₚₗₑ₂)) were diluted with 5% skimmed milk in 3-fold series starting from 10 μg/mL. Plates were washed using wash buffer (10 mM Na₂HPO₄, 500 mM NaCl, 0.1% Tween-20, pH 7.2) and incubated 1 hour with primary pAb at room temperature with agitation. Followed by wash with wash buffer, and incubation with secondary pAb (anti-rabbit IgG, P0448) diluted in 5% skimmed milk to 10 μg/mL for 1 hour with agitation. Finally, plates were washed with washing buffer and developed for 5 minutes after adding TMB (DAKO S1599) 100 μL per well. Reactions were stop by adding 100 μL 0.5 M H₂SO₄ to each reaction well. Results were detected by ELISA reader using SOFTMAX™ 6.2.1 with detection wavelengths 450 nm and 650 nm.

### Agglutination Assay

Turbidimetric assays were carried out using an ABX Pentra400 (HORIBA) instrument to measure increasing agglutination over time. The instrument wavelengths were adjusted to 340 and 700 nm in order to probe aggregates resulting during the agglutination reaction. The analytic cup contained 154 μl S2007 Buffer (DAKO), 8 μL antigen, 31 μL pAb and 5 μL H₂O. Resulting in a final reaction volume of 198 μL. Antigens were diluted to 2 mg/mL (human IgG, 0.54 μM final concentration) or 1 mg/mL (rabbit IgG, 0.27 μM final concentration) and from here diluted 2 times for each dilution step with a final of nine concentrations. All agglutination experiments were carried out at a pAb concentration at 10 mg/mL (10.5 μM final concentration) to ensure that also low populated IgG subpopulations are in measurable range.

### Example 3

This example demonstrates exemplary methods for making and using chimeric or recombinant immunogens as provided herein.

In this example, the number of potential epitopes in the antigen (or immunogen) was lowered. For example, a number of hydrophobic epitopes have been removed with the intention of developing a polyclonal antibody with less hydrophobic paratopes. These changes will make the polyclonal antibody more useful for particle enhanced turbidimetry.

### Material and Methods

### Epitope Selection

Human and rabbit Serum Amyloid A1 (SAA1) sequences were fetched from databases (Uniprot: PODJI8 and P53614, respectively). Sequences were aligned to identify sequence differences in the hydrophilic region between the two species. The selection of epitopes was done by aligning and inspecting the crystal structures of human SAA1 (PDB entry: 4IP8). Residues involved in hydrogen bond were not selected.

### Protein Expression and Purification

Constructs encoding chimeric variant of SAA1 or rabbit SAA were order at GENSCRIPT® as plasmids cloned into pET30a(+). The constructs were designed with an N-terminal His-tag followed by a TEV cleavage site in order to separate the His-tag from the SAA1 domain. Recombinant rabbit SAA1 domain, and chimeric SAA1 domain variant with human epitopes grafted onto the rabbit scaffold, were expressed in inclusion bodies using E. coli strain BL21 (Invitrogen™). Cell were harvested and resuspended in 8 M urea, 20 mM Tris, pH 8.5, spun and filtered with 0.2 p.m filters before recombinant protein were immobilized by affinity chromatography (IMAC) using a His-tag column (GE Healthcare). Immobilized protein was washed with wash buffer (20 mM Tris-HCL, 1 M NaCl, 20 mM Imidazole, pH 8.5) with at least 15 column volumes, and eluted with elution buffer (20 mM Tris-HCL, 150 mM NaCl, 500 mM Imidazole, pH 8.5). His-tagged recombinant protein was dialyzed against 20 mM Tris-HCl pH 8.5 buffer before adding TEV protease (0.2 mg/mL, final concentration) supplemented with 2 mM dithiothreitol (DTT, final concentration). The cleavage reaction was left for minimum 4 hours before separating cleaved recombinant protein from His-tag, TEV protease, and un-cleaved recombinant protein by loading mixture onto His-tag column. Flow through containing the untagged recombinant protein was collected. The sample was unfolded by dialyses against binding buffer, and further purified using size exclusion chromatography (SEC) SUPERDEX 75 PREP GRAD™ (GE Healthcare) with binding buffer as eluent. Finally, the SEC purified recombinant protein was refolded by dialysis against 20 mM Tris, pH, and used as antigen. For analysis (ELISA—and agglutination assays) recombinant proteins could retain the His₈-tag.

### SDS-PAGE and Western Blotting

Protein purity was followed by SDS-PAGE using pre-caste NuPage 4-12% Bis-Tris gels (Invitrogen™). All protein samples were loaded with SDS sample buffer (350 mM Tris.HCL, 357 mM Sodium dodecyl sulfate, 44.6% Glycerol, 179 μM Bromophenol blue, pH 6.8) and carried out in a MES SDS running buffer (NOVEX®). Gels were stained with SimplyBlue™ (Invitrogen™). For Western blotting NuPage 4-12% Bis-Tris gels (Invitrogen™) and MES SDS running buffer (Novex®) were used to separate the proteins. The electro blotting was carried out at 30 V for 1 hour, and proteins were transferred to PVDF membrane (BioRad) in Western blot buffer (25 mM Tris, 0.192 M Glycine and ethanol 25.3%). The blotting was followed by a blocking step using blocking buffer (50 mM Tris-HCL, 0.5 M NaCl, 0.5% Tween20, pH 9.0). Blocked PVDF membrane containing transferred protein was incubated for 1 hour (minimum) with primary pAb (rabbit anti-E. coli diluted 1000×) overnight at +4° C. with shaking. Before membrane was incubated for 1 hour with secondary pAb (swine anti-rabbit) the membrane was washed 4×10 min. with blocking buffer. After incubation with secondary pAb membrane was washed 4×10 min. with blocking buffer and incubated 20 min with DAB (diaminobenzidine) plus substrate.

### Antigen Preparation and Immunization

Based on SDS-PAGE and Western blotting analysis of fractions from SEC purification a highly pure antigen sample was produced. Only fractions without impurities were included in the final antigen sample. Antigen sample were mixed in equally amount (1:1) with Freunds incomplete adjuvant (FIA) just before subcutaneously immunization of three-month-old rabbits. Sera were collected before immunization and seven weeks after last immunization. Sera were preserved by adding sodium azide (NaN₃) final concentration 15 mM and stored a 4° C.

Transfer of Human Epitopes onto the Rabbit Scaffold:

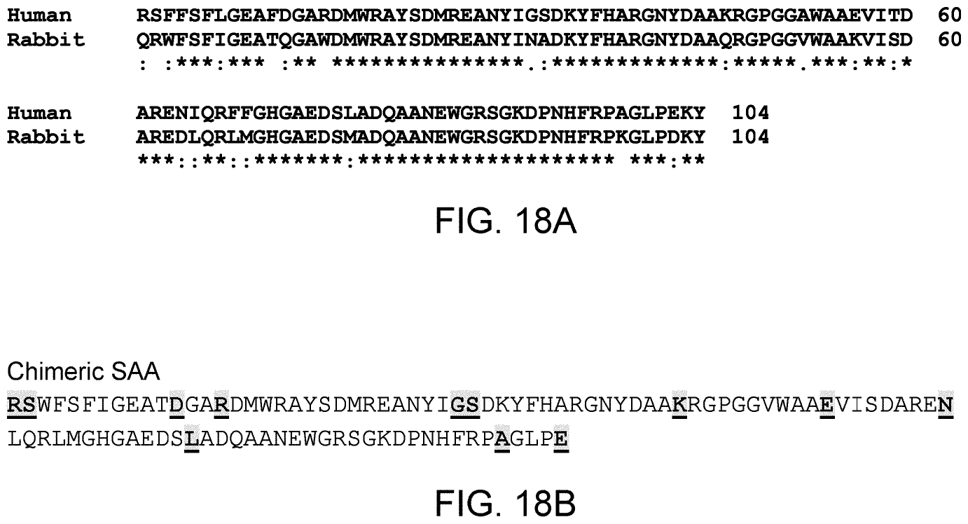

The human—and rabbit Serum Amyloid A (SAA) sequences differences were found using sequence alignment, as illustrated in FIG. 18A, with the human sequence as SEQ ID NO:9, and the rabbit sequence as SEQ ID NO:10. FIG. 18B illustrates the chimeric Serum Amyloid A (SAA) sequence (SEQ ID NO:11) showing the selected epitopes (black, underlined and in bold) with species specific sequence that were situated in the hydrophilic region grafted onto a rabbit backbone (red) sequence.

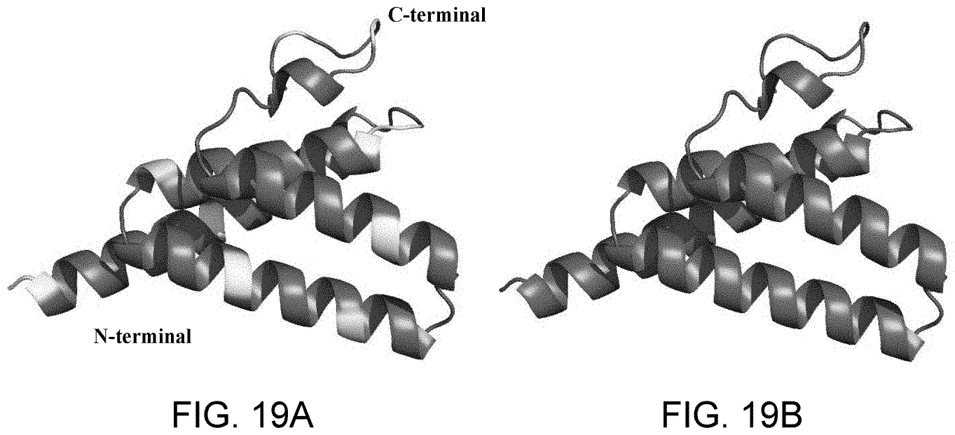

FIG. 19 schematically illustrates images of SAA, with the left-hand image showing human epitopes (yellow) grafted onto the rabbit backbone (red) sequence.

Synthesized sequence was inserted into expression vectors. Chimeric protein was expressed in E. coli and purified before immunization.

### Example 4

This example demonstrates exemplary methods for making and using chimeric or recombinant immunogens as provided herein.

In this example we have made a construct designed to cause immunized rabbits to make polyclonal antibody against the hidden epitopes in the human kappa light chain constant domain. Such an antibody is expected to be specific for human kappa free light chain and can therefore be used for diagnostic purposes on samples from, for example, myeloma patients.

### Material and Methods

### Epitope Selection

Human and rabbit Kappa free light chain (κ-FLC) sequences were fetched from databases (Uniprot: P01834 and P01840, respectively). Sequences were aligned to identify sequence differences in the κ-FLC constant domain between the two species. The selection of epitopes was done by aligning and inspecting the crystal structures of human κ-FLC (PDB entry: 6n35) and intact human IgG (PDB entry: 1hzh). PISA server was used to identify surface epitopes with less than 10% solvent exposed residues. These residues were included in the chimeric variants to produce a specific κ-FLC pAb.

### Protein Expression and Purification

Constructs encoding chimeric variant of κ-FLC or rabbit κ-FLC (B4) were order at GENSCRIPT® as plasmids cloned into pET22(+). The constructs were designed with an N-terminal His-tag followed by a TEV cleavage site in order to separate the His-tag from the κ-FLC constant domain. Recombinant rabbit κ-FLC constant domain, and chimeric constant domain variant with human epitopes grafted onto the rabbit scaffold, were periplasmic expressed using E. coli strain BL21 (Invitrogen™). The lysogeny broth medium containing recombinant protein was dialyzed against binding buffer (20 mM Na₂HPO₄, 150 mM NaCl, pH 7.4) before recombinant protein were immobilized by affinity chromatography (IMAC) using a His-tag column (GE Healthcare). Immobilized protein was washed with wash buffer (20 mM Na₂HPO₄, 1 M NaCl, 20 mM Imidazole, pH 7.4) with at least 15 column volumes, and eluted with elution buffer (20 mM Na₂HPO₄, 150 mM NaCl, 500 mM Imidazole, pH 7.4). His-tagged recombinant protein was dialyzed against binding buffer before adding TEV protease (0.2 mg/mL, final concentration) supplemented with 2 mM reduced L-Glutathione (final concentration). The cleavage reaction was left 4 hours before separating cleaved recombinant protein from His-tag, TEV protease, and un-cleaved recombinant protein the mixture was loaded onto His-tag column, and flow through was collected and contain the untagged recombinant protein. The sample was further purified using size exclusion chromatography (SEC) SUPERDEX 75 PREP GRAD™ (GE Healthcare) with binding buffer as eluent. For analysis (ELISA—and agglutination assays) recombinant proteins could retain the His₈-tag.

### SDS-PAGE and Western Blotting

Same as described in Example 1.

### Antigen Preparation and Immunization

Based on SDS-PAGE and Western blotting analysis of fractions from SEC purification a highly pure antigen sample was produced. Only fractions without impurities were included in the final antigen sample. Antigen sample were mixed in equally amount (1:1) with Freunds incomplete adjuvant (FIA) just before subcutaneously immunization rabbits. Sera were preserved by adding sodium azide (NaN₃) to a final concentration of 15 mM and stored a 4° C.

### Transfer of Human Epitopes Onto the Rabbit Scaffold

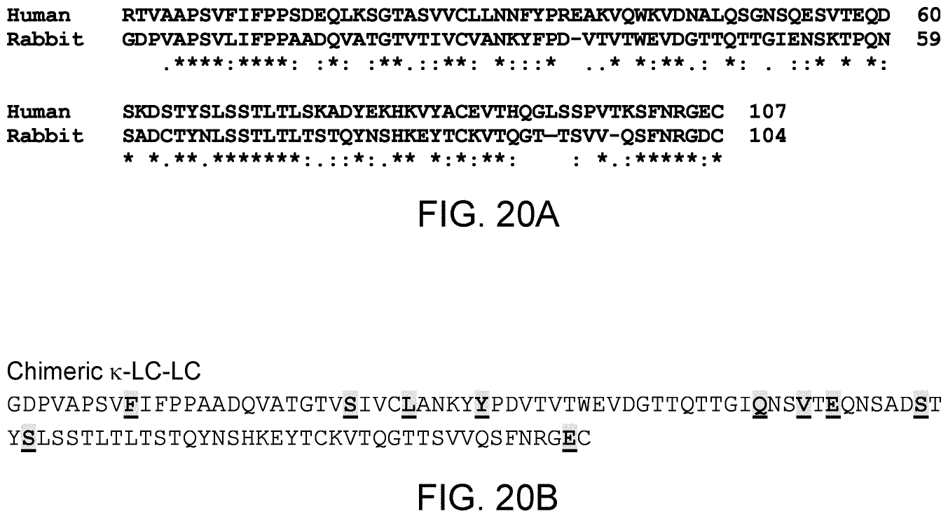

FIG. 20A shows how human (SEQ ID NO:12) and rabbit (SEQ ID NO:13) κ-FLC constant domain sequences differences were found using sequence alignment.

FIG. 20B illustrates a chimeric sequence (SEQ ID NO:14) with the selected epitopes (black, underlined and bolded) grafted onto the rabbit backbone (blue) sequence.

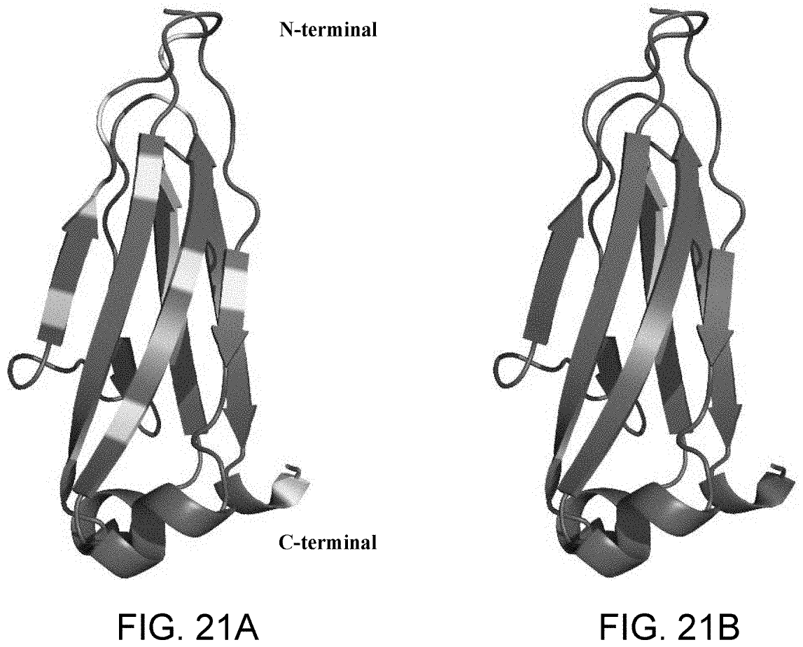

FIG. 21 illustrates the epitopes (in yellow in the left-hand image) which were selected, these epitopes were species specific sequence that are situated in selected hidden region.

The chimeric sequences were synthesized and inserted into expression vectors.

### Preparation of Antigen

Expression constructs was transformed into cells from a relevant organism and used for production of recombinant protein. The protein was purified using standard methods such as HIS-trap columns and size exclusion columns (SEC).

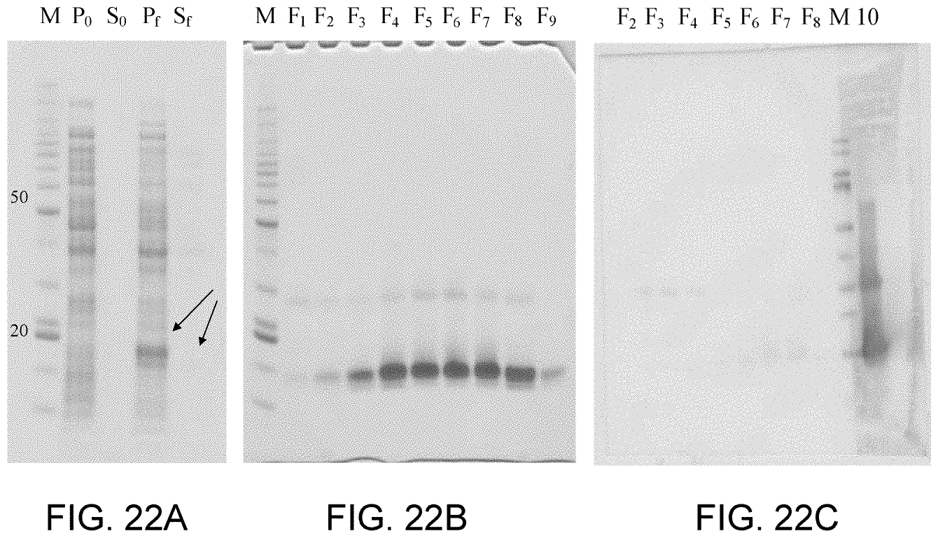

FIG. 22A illustrates an SDS-PAGE showing protein expression to demonstrate overexpression of protein with the expected band at approximately 13 kDa to 14 kDa (arrows) in both pellet (P) and supernatant (S).

FIG. 22B illustrates SDS-PAGE showing protein purity after TEV cleavage and SEC;

FIG. 22C illustrates a Western blot (WB) showing protein purity after TEV cleavage and SEC.

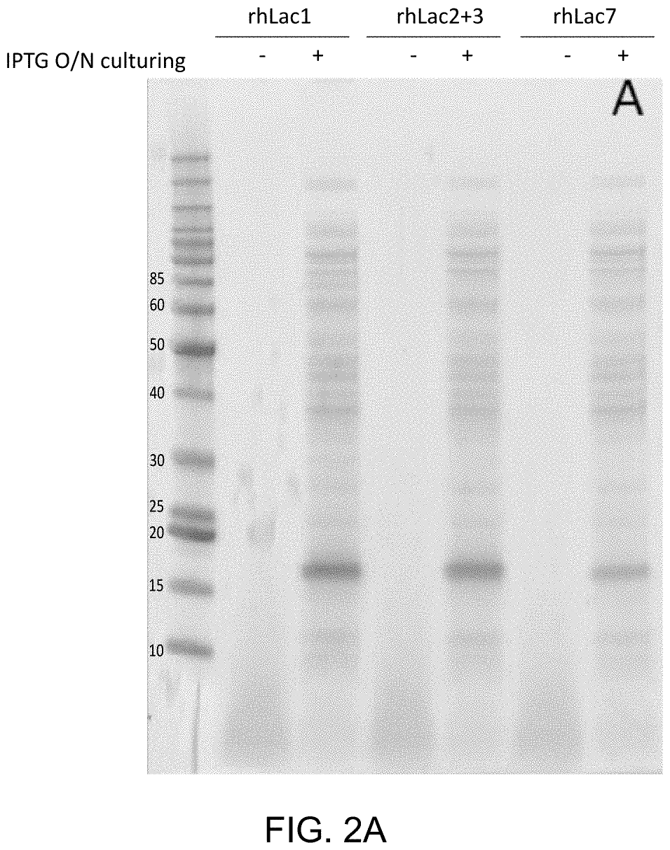

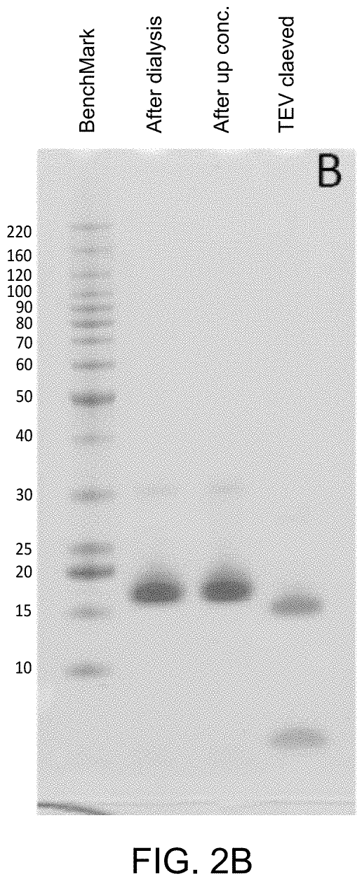

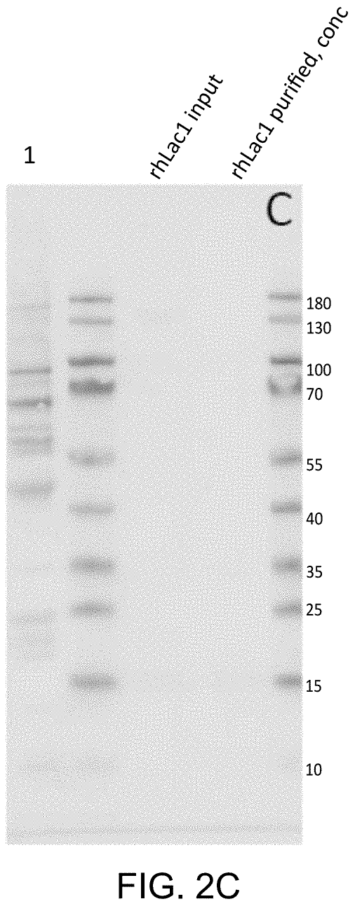

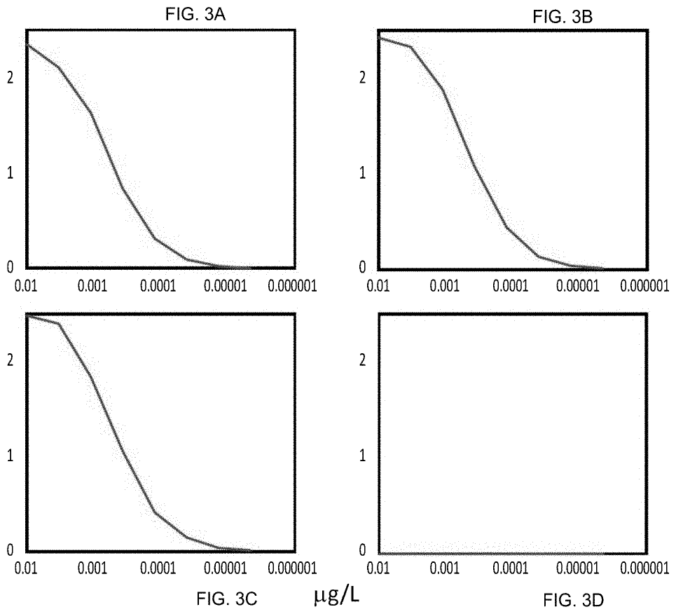

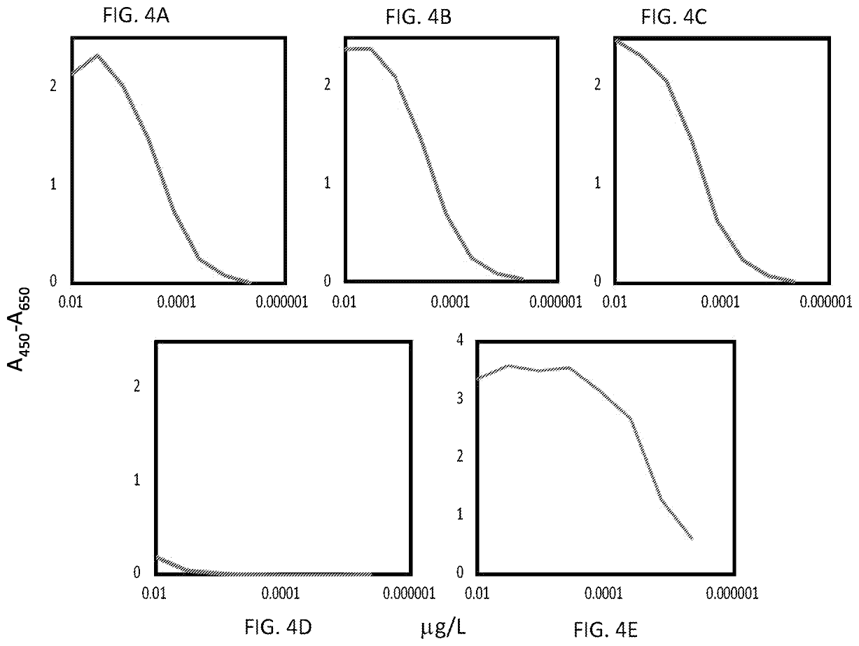

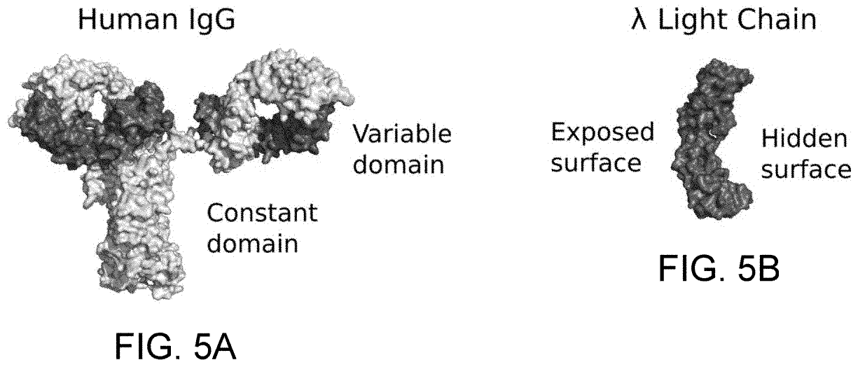

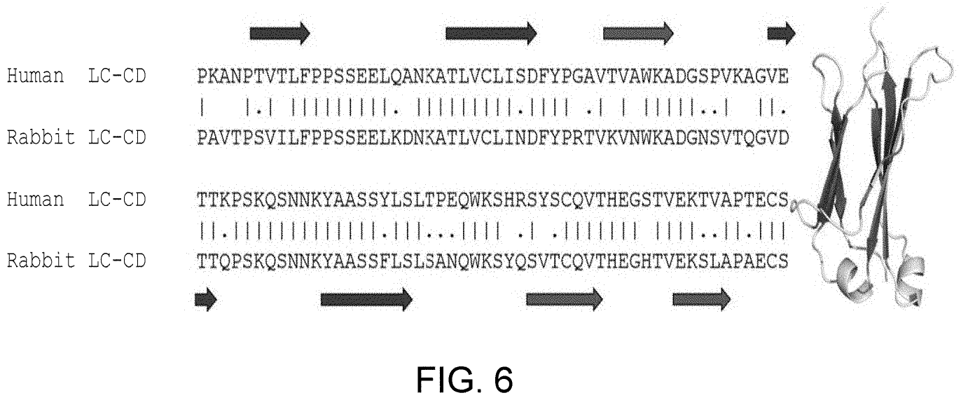

Based on SDS-PAGE and WB, pure fractions (Fₓ) were pooled and constituted the antigen. In the Western blot DAKO A0100 a polyclonal κ-FLC product was used as positive control (10) to demonstrate functionality of the chimeric antigen. Functionality of anti-E. coli pAb used for verification of sample purity has been shown previously (see λ-FLC example FIG. 2C).

For size determination in SDS-PAGE BENCHMARK™ Molecular weight (220, 160, 120, 100, 90, 80, 70, 60, 50 40, 30, 25, 20, 15, 10 kDa) was used, and in WB PAGERULER™ pre-stained protein ladder (180, 130, 100, 70 (orange), 55, 40, 35, 25, 15, 10 (green) kDa) was used.

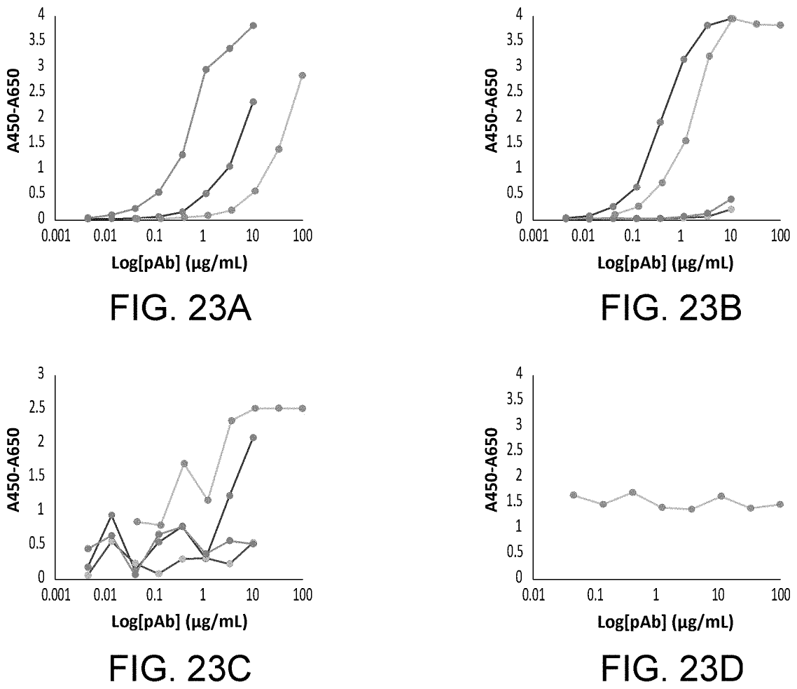

FIG. 23 illustrates ELISA data showing that antiserum from rabbit immunized with chimeric rhκ-Cd are specific with no or little reactivity against recombinant rabbit kappa constant domain (D), but with reactivity against human κ-FLC (B). The antiserum also shows less reactivity against intact human IgG (A) indicating more specific than the control A0100.

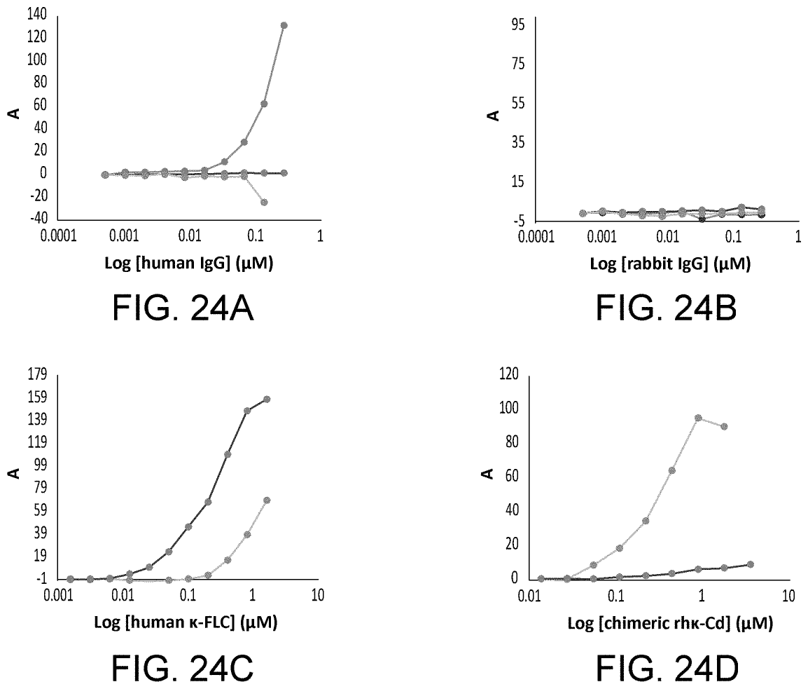

FIG. 24 illustrate agglutination experiments showing that antiserum are able to agglutinate human κ-FLC (C), but not intact human IgG. Demonstrating that antiserum react with more than one epitope and that these epitopes are hidden in human intact IgG.

### Example 5: Making Chimeric Antigens

This example demonstrates exemplary methods for making and using chimeric immunogens as provided herein.

For the human IgG subtypes 1-4 we designed antigens according to methods as provided herein, and produced the antigens recombinantly and purified them using chromatography.

### Material and Methods

### Epitope Selection

Human IgG isotypes (1, 2, 3 and 4) and rabbit IgG sequences were aligned to identify isotypic sequence differences in the Ch1-Ch2-Ch3 region of IgG. By inspecting the crystal structure of whole IgG (pdb entry: 1hzh) the epitopes were carefully selected.

### Protein Expression and Purification

Standard HEK cell expression followed by Protein A or Protein G purification and further purified by SEC using PBS as eluent.

### SDS-PAGE and Western Blotting

Same as described in Example 1.

### Antigen Preparation and Immunization

Based on SDS-PAGE and Western blotting analysis of fractions from SEC purification a highly pure antigen sample was produced. Only fractions without impurities were included in the final antigen sample. Antigen sample were mixed in equally amount (1:1) with Freunds incomplete adjuvant (FIA) just before subcutaneously immunization of three-month-old rabbits. Sera were collected before immunization and seven weeks after last immunization. Sera were preserved by adding sodium azide (NaN₃) final concentration 15 mM and stored a 4° C.

### Transfer of Human Epitopes Onto the Rabbit Scaffold:

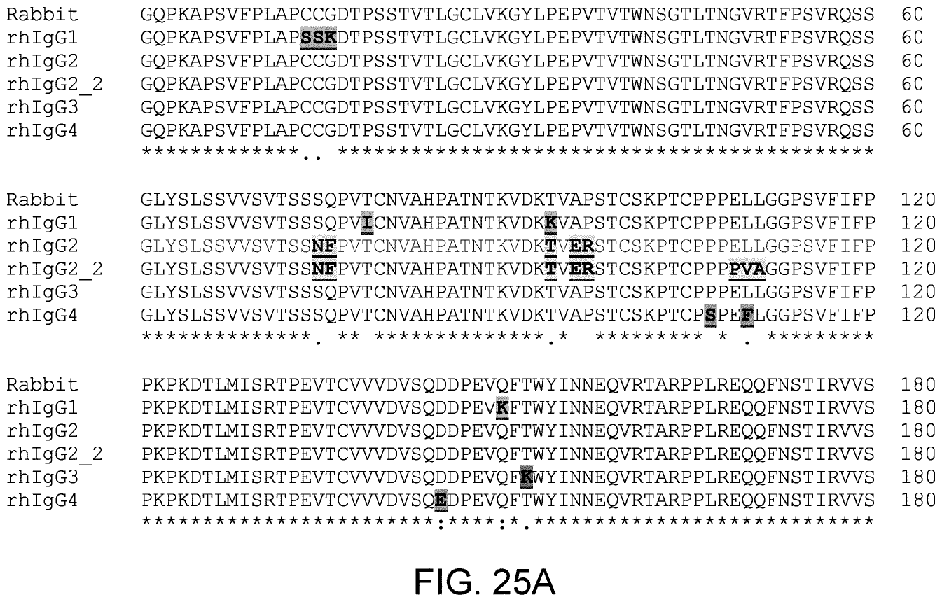

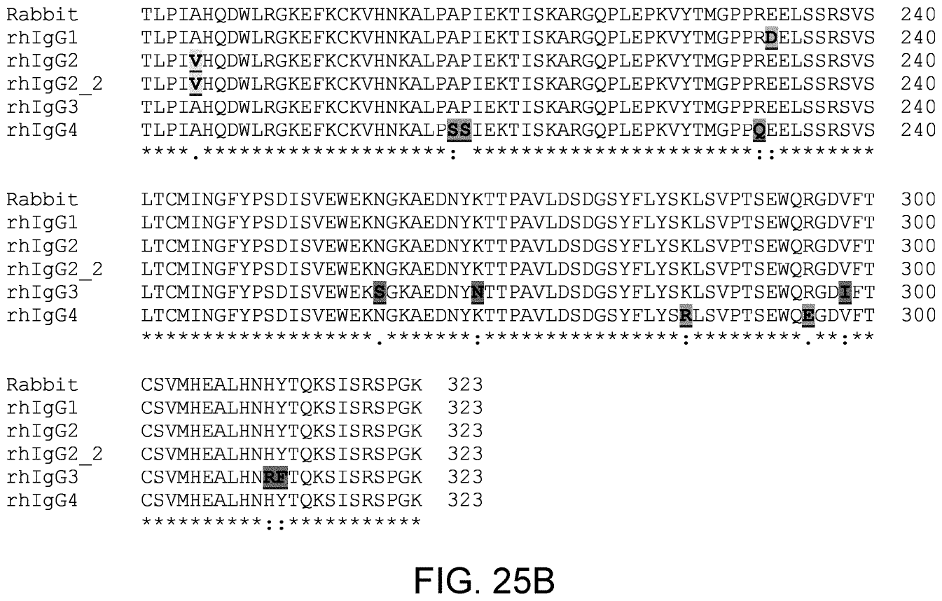

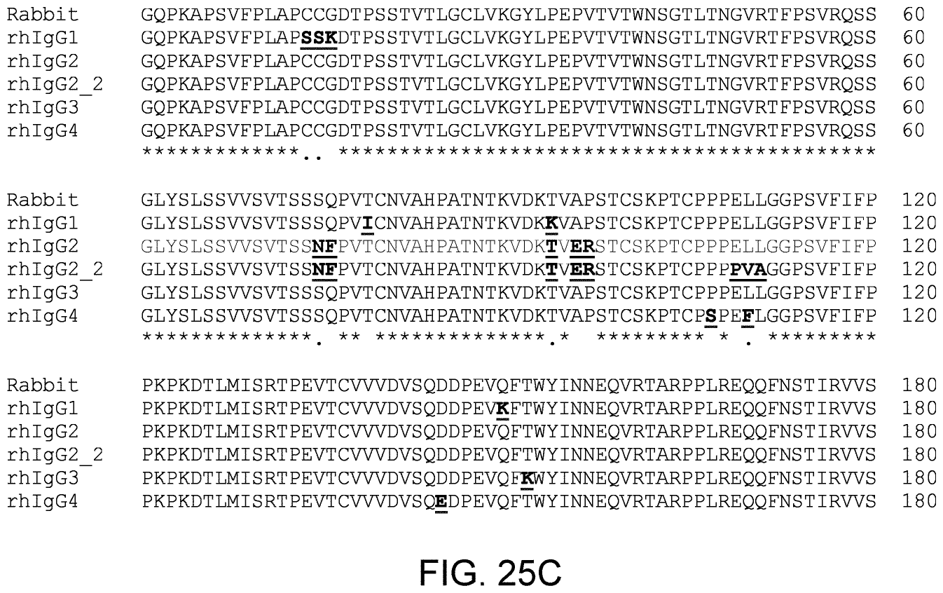

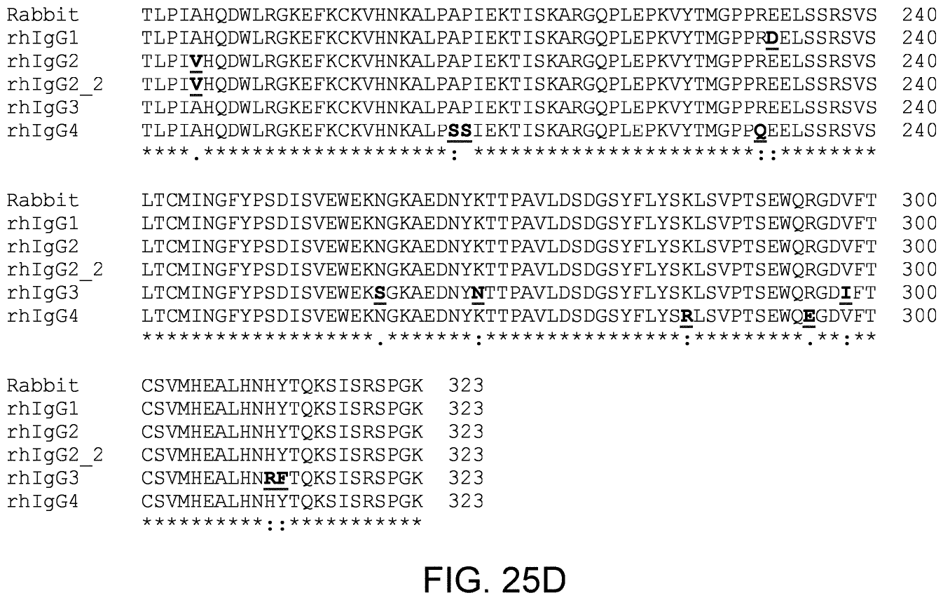

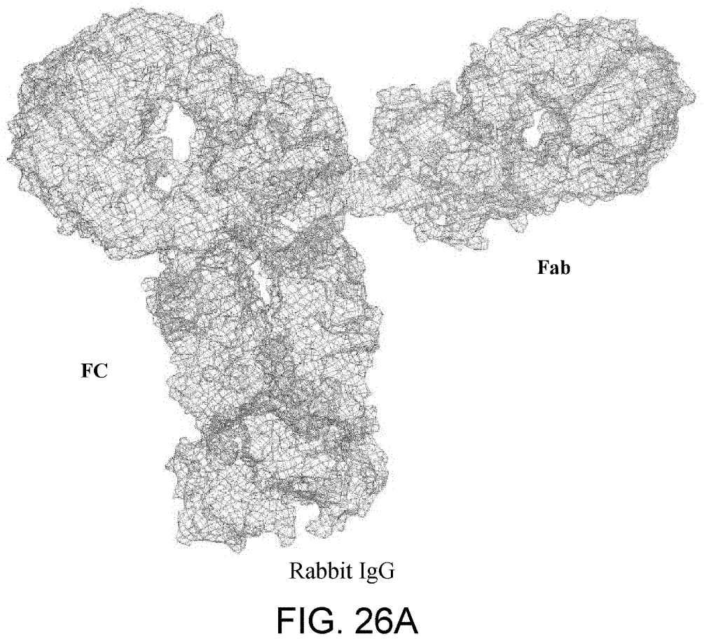

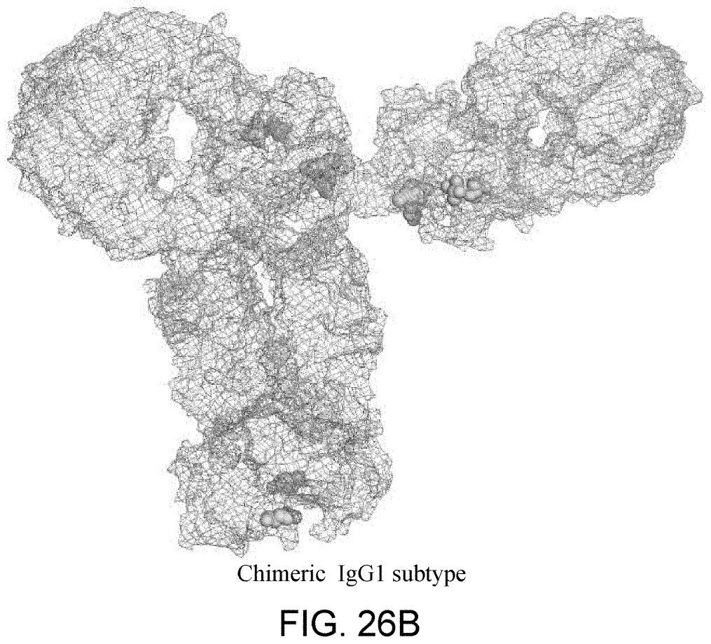

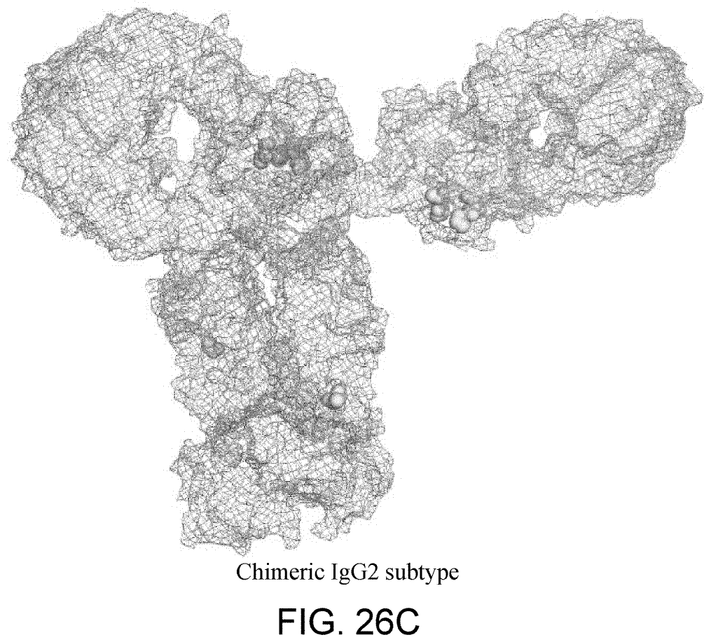

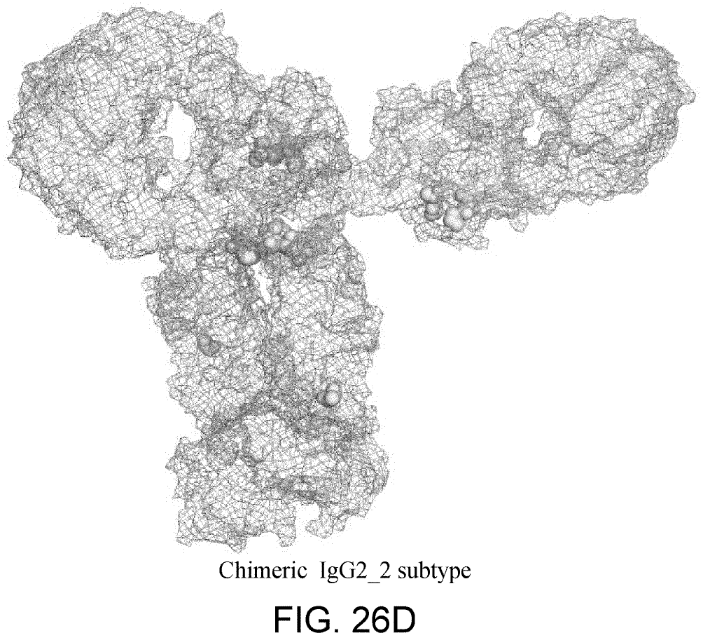

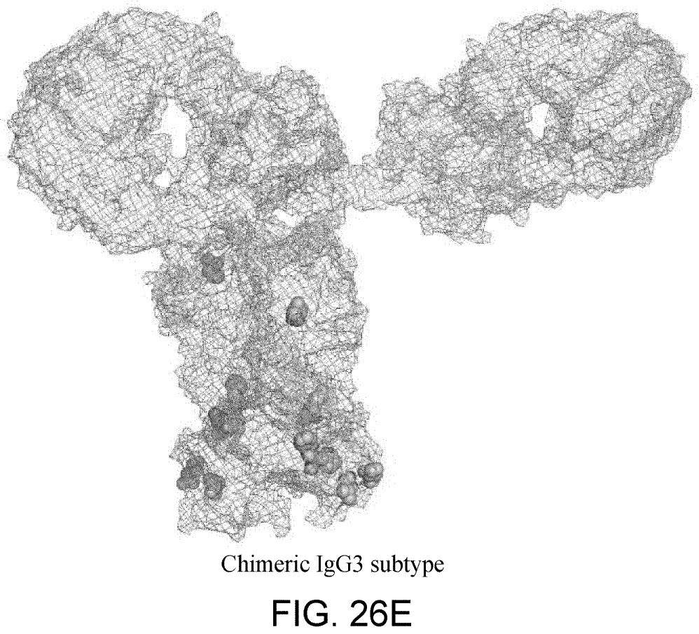

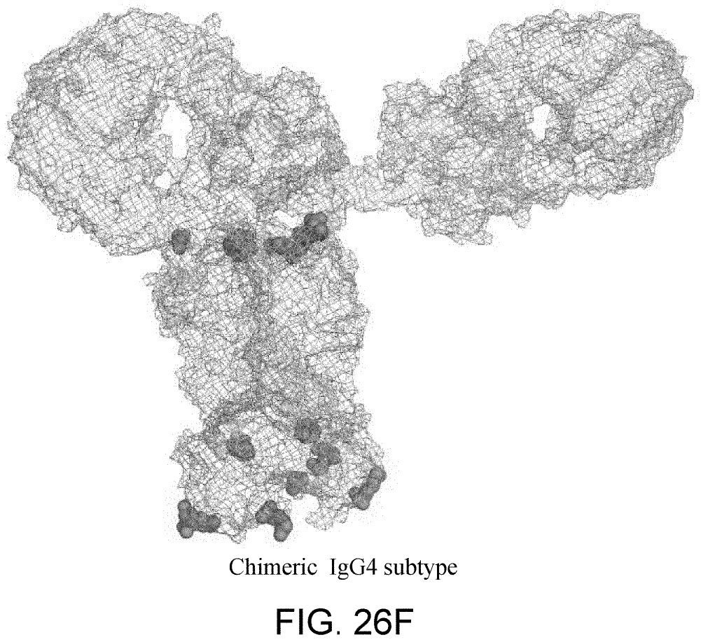

FIG. 24A-B illustrates how human—and rabbit gamma immunoglobulin (IgG) sequences differences were found using sequence alignment; chimeric sequences with the selected epitopes (FIG. 24A: colored and underlined; FIG. 25B, underlined and bolded) were grafted onto the rabbit backbone sequence, and were synthesized and inserted into expression vectors; rabbit backbone (SEQ ID NO:15); rhIgG1 (SEQ ID NO:16); rhIgG2 (SEQ ID NO:17); rhIgG2_2 (SEQ ID NO:18); rhIgG3 (SEQ ID NO:19); rhIgG4 (SEQ ID NO:20).

FIG. 25A-F illustrate rabbit and chimeric IgG made by methods as provided herein, where human isotypic specific epitopes (colored) were grafted onto rabbit IgG backbone (grey); where FIG. 25A illustrates a rabbit IgG backbone without human epitopes inserted; and FIG. 25B illustrates a chimeric IgG1 subtype with human epitopes inserted (colored), FIG. 25C illustrates a chimeric IgG2 subtype with human epitopes inserted (colored), FIG. 25D illustrates a chimeric IgG2_2 subtype with human epitopes inserted (colored), FIG. 25E illustrates a chimeric IgG3 subtype with human epitopes inserted (colored), and FIG. 25F illustrates a chimeric IgG4 subtype with human epitopes inserted (colored).

### Example 6: Preparation of Recombinant Human Epitopes

In this example we demonstrate that human epitopes can be substituted onto a non-human polypeptide background, in this example, an immunoglobulin background, and specifically in this example a rabbit IgG background, to confer isotype specificity, or human IgG1, IgG2, IgG3 or IgG4 subtype specificity, of the antibody response by the immunized animal (in this example, in an immunized rabbit). In other words, for example, when human IgGlepitopes were generated in the rabbit immunoglobulin polypeptide (Ig) by substituting amino acids from human IgG1 for the rabbit amino acids, the antibody response by the animal immunized with this chimeric Ig was specific to the human IgG1 isotype Ig. The substituted human epitopes replaced rabbit epitopes, in other words, human epitopes substituted for selected rabbit epitopes.

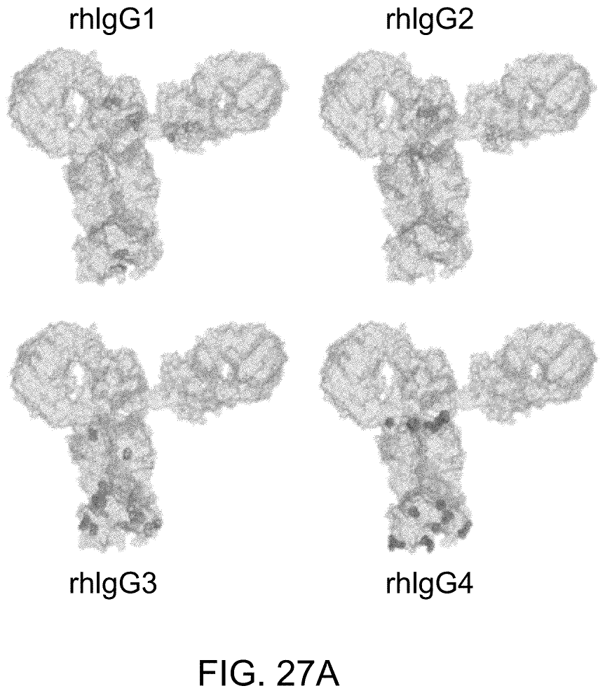

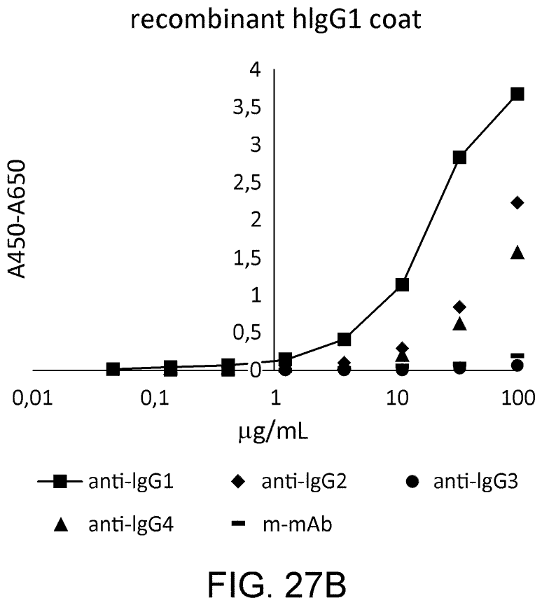

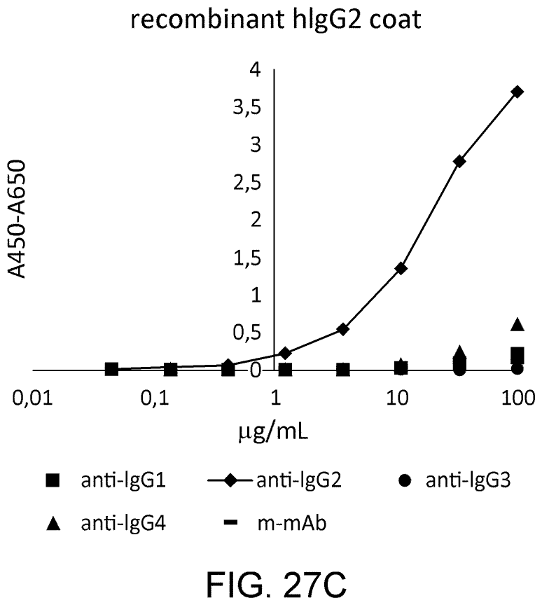

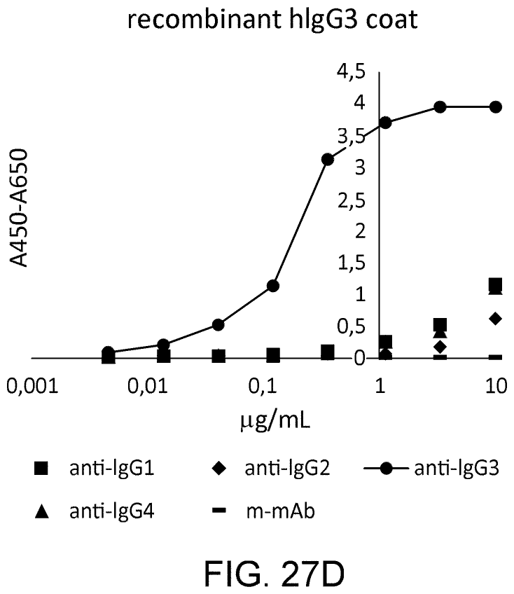

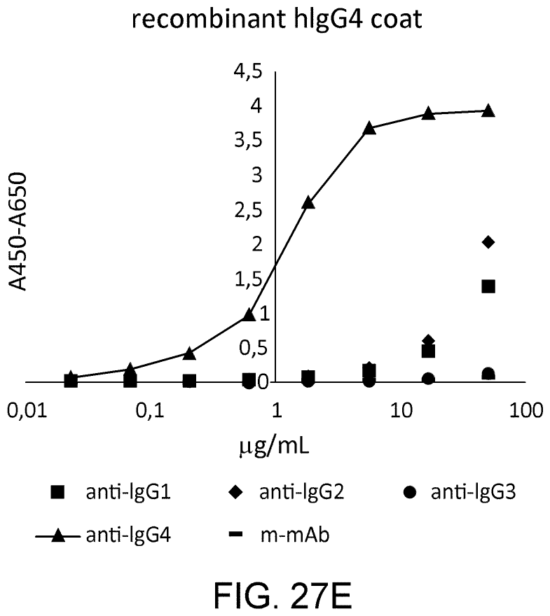

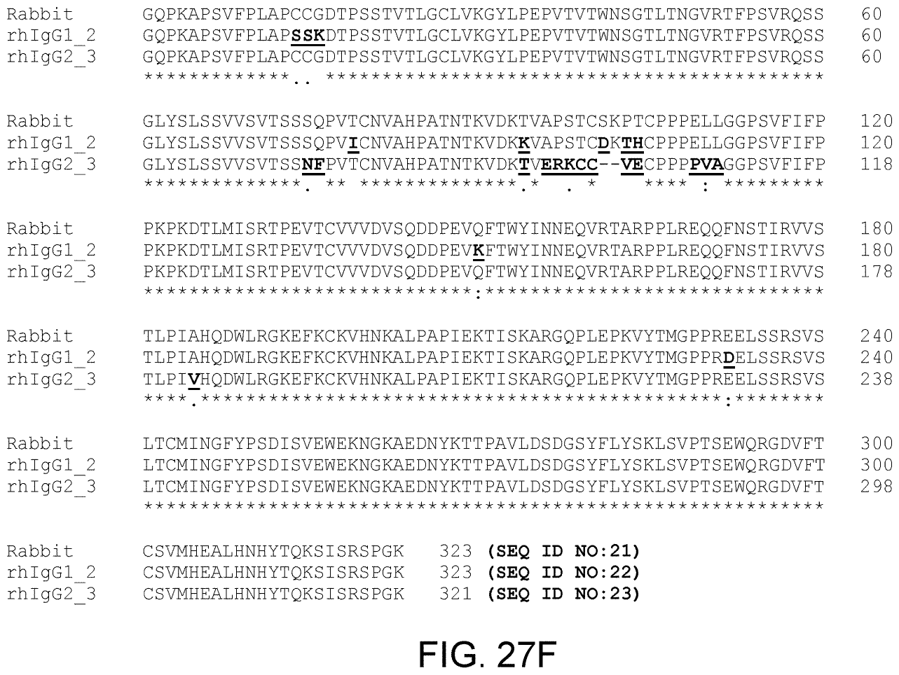

As illustrated in FIG. 27A, rabbit IgG carrying (or having substituted therein) epitopes specific for human IgG1, IgG2 IgG3 and IgG4 were constructed; the human epitopes (the human amino acid residues substituted in the rabbit Ig) are illustrated in darkened colors.

As graphically illustrated, FIG. 27B-E show the rabbit immune response of human IgG1, IgG2 IgG3 and IgG4 epitopes in rabbit Ig, respectively. In all four cases (human IgG1, IgG2 IgG3 and IgG4 epitopes), a similarly low level of background (dots) was seen. In contrast, the specific signal (solid titration curve) was high in rabbits immunized with rabbit IgG carrying epitopes specific for human IgG3 or IgG4 and the specific signal was lower (or medium) in rabbits immunized with rabbit IgG carrying epitopes specific for human IgG1 or IgG2.

Together these data demonstrate that human epitopes, including by not limited to human Ig isotype epitopes, can be substituted onto a rabbit IgG background and result in a human-epitope specific immune response from the rabbit (for example, elicit a IgG1-4 subtype response specificity in immunized rabbits).

Because the specific signal was lower (or medium) in rabbits immunized with rabbit IgG carrying epitopes specific for human IgG1 or IgG2, the two IgG1 or IgG2 subtypes were re-designed to improve the rabbit's reactivity to the Ig1 and IgG2 epitopes; the amino acid sequences of the revised variants of chimeric IgG1 and IgG2 subtype immunogens are shown in FIG. 27F, with underlined amino acid residues representing human epitopes.

### Example 7: Remove Selected Epitopes

In this example we demonstrate how removal of specific epitopes (or substitution of one type of epitope for another epitope) leads to improved performance of the resulting generated antibodies; specifically, it was shown that removal of selected human hydrophobic residues (which were substituted for non-hydrophobic residues) resulted in antibodies that were less hydrophobic, and thus less self-associating, and the decrease in self-association resulted in their improved performance when the less hydrophobic Ig were attached to beads in an agglutination reaction. In this Example, the substituted epitopes were inserted in a rabbit Serum Amyloid A (SAA) polypeptide background or backbone.

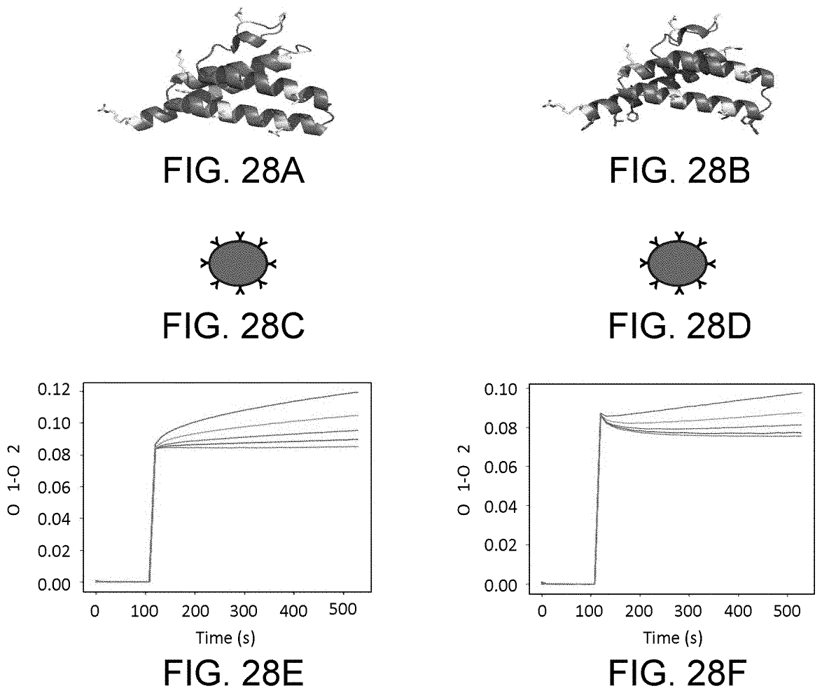

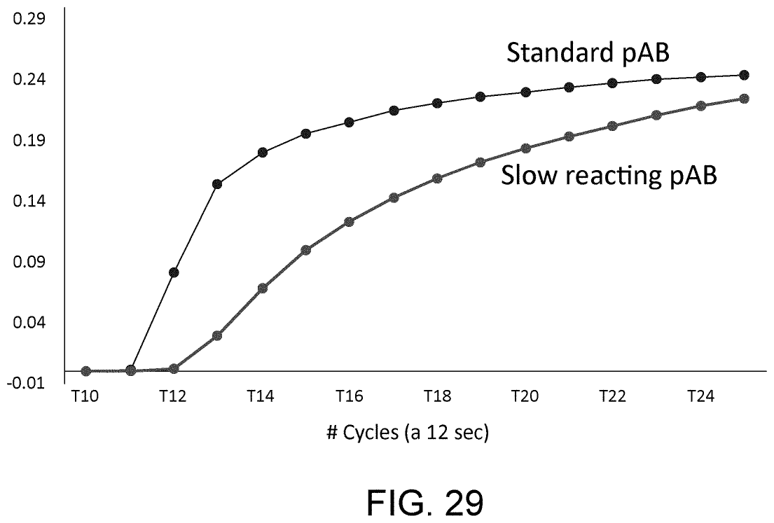

FIG. 28A illustrates a rabbit Serum Amyloid A (SAA) backbone, with red residues (or darker color) being the rabbit SAA sequence, and yellow (or lighter color) representing inserted human specific amino acids.

FIG. 28B illustrates a human SAA with blue (or darker) color, or circled residues, indicating six hydrophobic residues that may interact with a lipid surface.

Chimeric SAA were constructed with the six hydrophobic human (blue) moieties, or circled residues, replaced by a corresponding rabbit amino acid, as shown in FIG. 28A.

FIG. 28C illustrates antibody derived from immunization with the SAA illustrated in FIG. 28A coupled to beads.

FIG. 28D illustrates antibody derived from immunization with the SAA as illustrated in FIG. 28B leads to immune particles having antibodies (Abs) comprising some paratopes that recognizes the human hydrophobic (blue) epitopes.

FIG. 28E-F are diagrams showing the kinetics of C and D reacting to five different levels of SAA.

For FIG. 28A-B, amino acid residues that are different in human SAA as compared to rabbit SAA are shown in yellow (or lighter residues) and blue (or darker, and circled, residues). In FIG. 28B the six blue (or darker, and circled) residues are hydrophobic epitopes potentially involved in binding of SAA to lipid particles.

When conjugating the SAA polyclonal antibody to a latex particle (FIG. 28D) the surface includes paratopes specific for the hydrophobic epitopes (indicated by blue color, or circled residues). When such beads are added to a reaction buffer containing SAA, an abnormal drop in OD is firstly observed. Thereafter the OD increases as the beads binds to SAA and increases the turbidity of the fluid.

In contrast, when using beads coupled to an antibody derived from immunization with SAA having rabbit sequence at the six blue, or circled residue positions (as illustrated in FIG. 28A), the OD increases immediately as is normal.

A suggested explanation is that the six hydrophobic residues induce antibody with some hydrophobic character of the paratopes. Due to these hydrophobic paratopes, the antibody labelled beads may become associated to some extent. When such beads are placed into the reaction buffer, they disperse causing a lowered absorbance until the agglutination reaction takes over and causes an increase in the absorbance.

The amino acid sequence of rabbit SAA with inserted human amino acid residues is below, with bolded residues indicating non-hydrophobic inserted human residues, and underlined residues indicating rabbit hydrophobic residues:

SEQ ID NO: 24
RSWFSFIGEATDGARDMWRAYSDMREANYIGSDKYFHARGNYDAAKRGPGG
VWAAEVISDARENLQRLMGHGAEDSLADQAANEWGRSGKDPNHFRPAGLPE
KY

### Example 8: Generation of Slow Reacting Epitopes

In this example we demonstrate construction of chimeric C-Reactive Protein (CRP) antigen substituted with only a few human specific amino acid (aa) residues, and that immunization with this modified chimeric polypeptide can lead to generation of a slow reacting antibody.

FIG. 29 graphically illustrates data demonstrating that immunization with incomplete human epitope can provide for a slow reacting polyclonal antibody (srpAb) to human CRP. Standard rabbit anti-human CRP polyclonal antibody in blue and srpAb in red. The srpAb was made by immunizing rabbits with rabbit CRP carrying an artificial epitope that is part human and part rabbit. srpAb was obtained from fully immunized rabbits having received 6 immunizations over 3 months. The two antibodies were titered to have same concentration of antibody to human CRP.

Human epitopes can be found that directs expression of a slow reacting anti-CRP antibody. The upper, or blue, curve shows absorbance kinetics when using DAKO polyclonal rabbit anti-human CRP antibody. The lower, or red, curve shows absorbance kinetics when using polyclonal rabbit anti-human CRP antibody derived from immunization using rabbit CRP with one human epitope. It will be appreciated that slow reacting epitopes can also be generated by inserting at least one amino acid from the second species or deleting at least one amino acid from the first species.

### Example 9: Constructing Polypeptide Epitopes on Ferritin Backbones

In this example we demonstrate construction of a chimeric molecule comprising a rabbit ferritin backbone, or core, with epitope-specific chimeric modules attached thereto via a linker.

In alternative embodiments, recombinant ferritin forms a 24-mer homomer that self assembles even when the N- or C-terminal encoding protruding rod is genetically altered to carry other protein sequences. Due to the virus particle-like structure with many repeats it causes very high levels of titer according to numerous publications.

In alternative embodiments, epitope specific chimeric modules are attached to a rabbit ferritin backbone such that they protrude out from the ferritin core, or ball, which can comprise 24 recombinant ferritin molecules. Functionally, the rabbit ferritin is a “silent” carrier (in that no immune reaction is generated to the ferritin core in rabbits, since the ferritin originates from the species being immunized) having fused thereto a plurality of chimeric proteins.

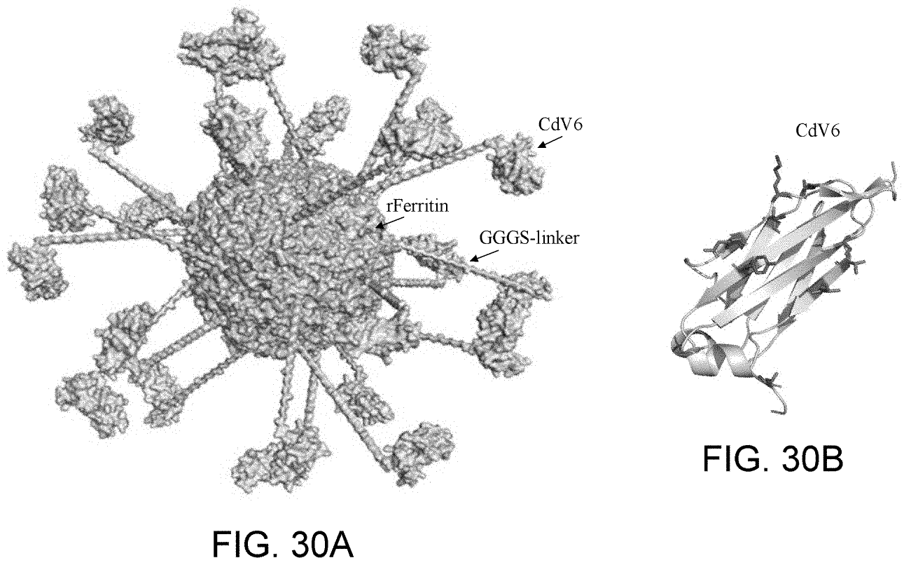

FIG. 30A-B illustrates an exemplary chimeric ferritin construct (FIG. 30A) comprising an attached immunoglobulin antigen CDv6 from rabbits which has been substituted with human epitopes, where the CDv6 is separately depicted in FIG. 30B. The chimeric CDv6-ferritin antigen can generate or direct high levels of polyclonal antibody to the human epitopes in CDv6, whereas the ferritin core or ball and the non-human sequences of CDv6 is non-immunogenic in the species being immunized (i.e., will not generate an antibody response). FIG. 30A illustrates an exemplary chimeric immunogen comprising a 24-mer rabbit ferritin fused with a chimeric rabbit CdV6 via a (GGGGS)₅ linker (SEQ ID NO:29), shown as a rod-like structure. FIG. 30B schematically illustrates the chimeric CdV6, which comprises a rabbit-human constant domain in gold (or lighter color) and with the inserted human epitopes colored red (or darker).

In alternative embodiments, the linker connecting each of the individual ferritin molecules of the ferritin “ball” or core to the antigen motif is selected to be as non-immunogenic as possible and/or be absent from proteins found in human samples to be analyzed with the derived polyclonal antibody. For example, the linker can be a polyG-comprising linker, for example, a GGGGS (SEQ ID NO:31) linker, or equivalents.

In alternative embodiments, the “silent”, or non-immunogenic, ferritin carrier protein is made with one of two components, such as coiled coil motifs that can bind to each other to form pairs and bind highly specifically together. When the second component (or coiled coil motif) is bound to an immunogenic polypeptide such as CDv6, it will “click” the CDv6 moiety onto coiled coil motifs on individual recombinant ferritin molecules in (or on the surface of) a ferritin ball or core such that the immunogenic polypeptide is displayed projecting away from the ferritin ball or core. This way a ferritin ball or core carrying up to 24 copies of an immunogenic polypeptide such as CDv6 comprising epitopes from a different species can be made and used for immunization and high titers of polyclonal antibody specific for the immunogenic polypeptide, for example, specific for the human epitopes inserted in the CDv6.

In alternative embodiments, the coiled coil motif comprises a gamma-aminobutyric acid type B receptor subunit 1 isoform X1 (GBR1) and/or gamma-aminobutyric acid type B receptor subunit 2 (GBR2)), where the GBR1 can selectively bind to GBR2 motif. In alternative embodiments, a GBR1 motif comprises:

(SEQ ID NO: 33)
STNNNEEEKSRLLEKENRELEKIIAEKEERVSELRHQLQSR.

In alternative embodiments, a GBR2 motif comprises:

(SEQ ID NO: 34)
SVNQASTSRLEGLQSENHRLRMKITELDKDLEEVTMQLQDT.

In alternative embodiments, the GBR1 motif is bound to the amino terminal of a ferritin molecule by use of a non-immunogenic linker, for example as shown below, where the GBR1 motif is underlined and the linker is bolded, and the remainder of the amino acid residues comprise the ferritin molecule:

(SEQ ID NO: 35)
N-terminal-STNNNEEEKSRLLEKENRELEKIIAEKEERVSELRHQLQ
SRGGGGSGGGGSGGGGSGGGGSGGGGSMTSQIRQNYSPEVEAAVNHLVNL
HLRASYTYLSLGFYFDRDDVALEGVSHFFRELAEEKREAAERLLKMQNQR
GGRALFQDVQKPSQDEWGKTLNAMEAALALEKNLNQALLDLHALGSAHTD
PHLCDFLENHFLDEEVKLLKKMGDHLTNIRRLSGPQASLGEYLFERLTLK
HD-C-terminal.

In alternative embodiments, a chimeric antigen is attached to the amino terminal of a coiled coil motif such as a GBR1 or GBR2 motif, optionally covalently attached by a non-immunogenic linker.

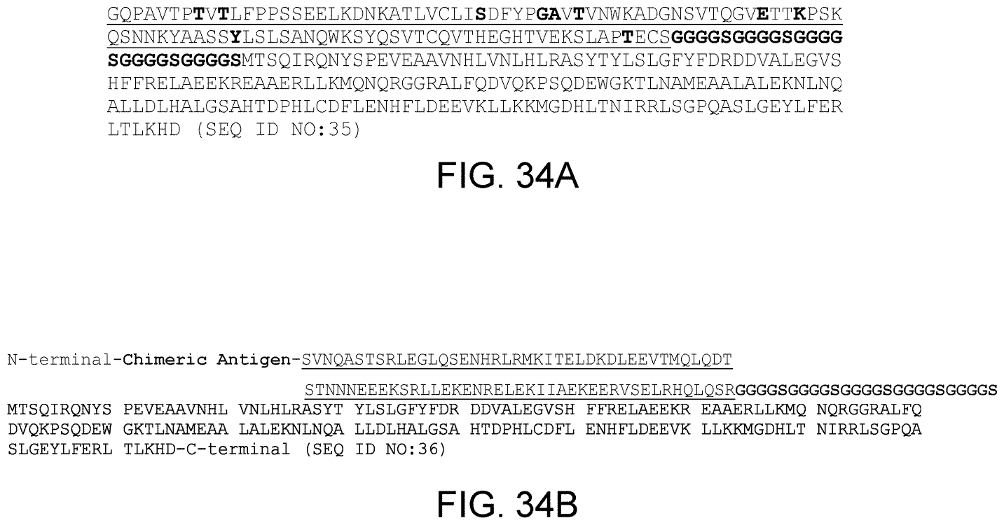

In alternative embodiments, the coiled coil motif (optionally a GBR1 or GBR2 motif) that is attached or linked to the chimeric antigen (optionally CDv6) binds (or is bound) to a coiled coil motif (optionally a GBR1 or GBR2 motif) covalently bound or linked to a ferritin molecule (optionally by using a non-immunogenic linker) to generate a heterodimer as illustrated in FIG. 34B.

In alternative embodiments, the need for further purification is unnecessary or minimal as the coiled-coil rabbit ferritin ball and the CDv6 backbone is from rabbit, and therefore immunologically silent in rabbit.

In alternative embodiments, the immunologically silent ferritin carrier protein is changed to carry sequences allowing site specific chemical modification. As one example, a His(6)-Lys-His(3) (SEQ ID NO:32) tag, known to confer an acetylation hot-spot, is inserted, with one His(6)-Lys-His(3) (SEQ ID NO:32) tag for each linker, attached to the linker distal to the ferritin core. Recombinant Ferritin-His(6)-Lys-His(3) (SEQ ID NO:32) is allowed to couple to activated moieties and a ferritin ball with up to 24 units of such protruding immunogenic moieties are formed and applied for immunization.

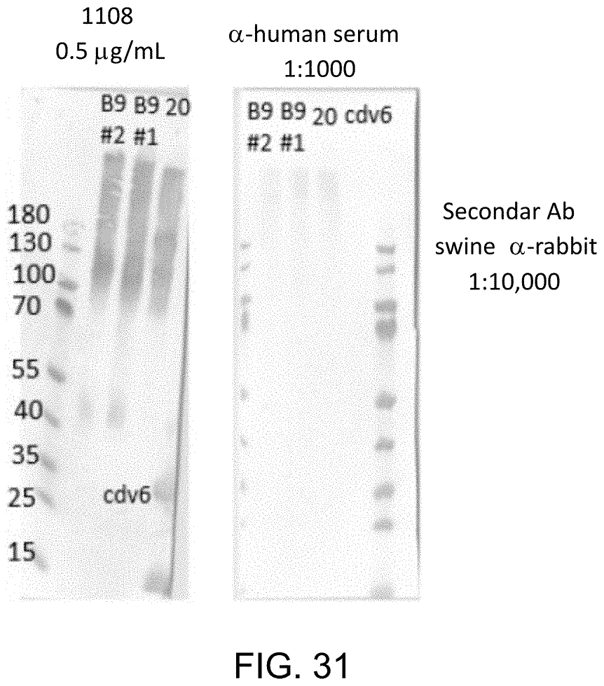

FIG. 31 illustrates an image of a Western blot showing that pAb used as primary IgG (sample 1108) elicited against immunogen CdV6, the chimeric rabbit human free light chain domain, interacts with the “B9” fraction from the size exclusion purification column (columns #2 and #1 are different purification fractions) and a “20 fraction” from a size exclusion, noting that anti-human serum has no or slightly reactivity against B9 and fraction 20, even though it is at 20 μg/mL; when 40 times more pAb was used, a slight reactivity is observed probably from cross reactivity with human Ferritin.

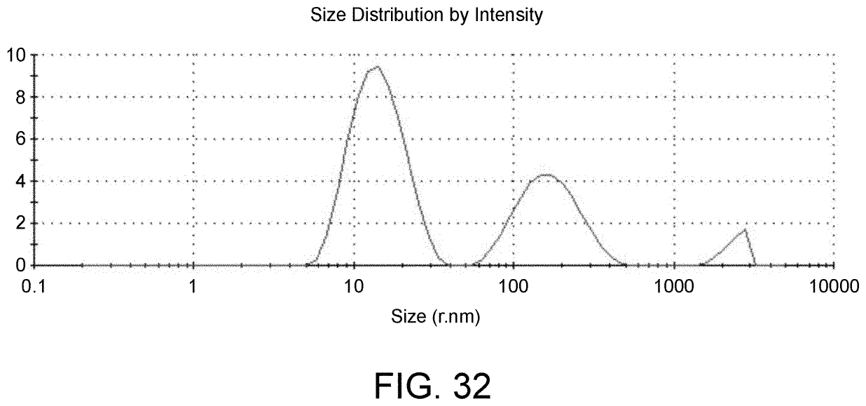

FIG. 32 graphically illustrates data showing dynamic light scattering (DLS), or size distribution by intensity (size being a function of intensity), the data showing that approximately 10 to 15 nm Rh is the primary particle size in the purified protein sample; this is in good agreement with the expected diameter of 20 to 30 nm when in counting linker and domain.

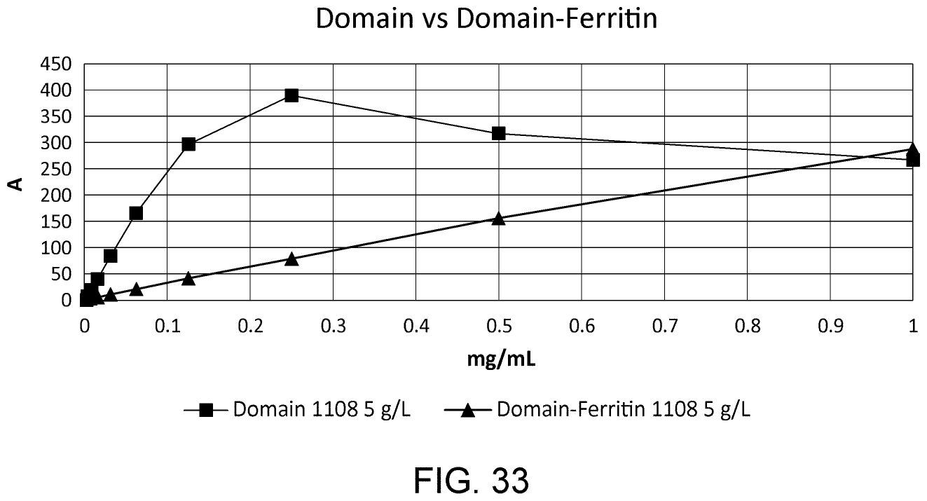

FIG. 33 graphically illustrates data showing that CDv6 expressed fused to Ferritin is correctly folded. Rabbit polyclonal antibody specific for human lambda epitopes recognizes the exemplary ferritin-CDv6 domain fusion protein. Since the polyclonal rabbit antibody specific for human lambda light chain epitopes both cross-linked the CDv6 domain as well as the ferritin-CDv6 fusion protein, when assembled into a 24 mer structure, it can be concluded that the CDv6 domain is correctly folded when fused to ferritin. The rate of agglutination is correlated to the size (radius) of the two proteins, suggesting that the rate is determined by diffusion.

FIG. 34A illustrates the sequence of an exemplary recombinant ferritin comprising a modified CdV6 having human epitopes inserted therein (SEQ ID NO:35), the underlined section are CdV6 sequence and the bolded residues are linker sequence, and the remainder of the sequence are rabbit ferritin residues.

FIG. 34B (SEQ ID NO:36) illustrates a heterodimer formed by the non-covalent binding of: (1) a chimeric recombinant antigen, where chimeric recombinant antigen is covalently bound to the amino terminal of a coiled coil GBR2 motif (SEQ ID NO:27); to (2) a GBR1 motif (underlined) (SEQ ID NO:26), which is bound to the amino terminal of a ferritin molecule by use of a non-immunogenic linker (bolded) (SEQ ID NO:28); where the two subunits of the heterodimer are non-covalently bound by the associate of the GBR1 motif to GBR2 motif. In one embodiment, 24 heterodimers join together to form a “ball” or core that displays to the external milieu the antigen; thus, when the 24-mer is administered as a immunogen it is effective in generating an immune response to the displayed antigen.

A number of embodiments of the invention have been described. Nevertheless, it can be understood that various modifications may be made without departing from the spirit and scope of the invention. Accordingly, other embodiments are within the scope of the following claims.

1. A chimeric or recombinant polypeptide comprising:
(a) a polypeptide derived from a first species, and
(b) at least one heterologous amino acid sequence or amino acid residue derived from at least a second species,
wherein the at least one heterologous amino acid sequence or amino acid residue derived from the second or additional species is inserted into, joined to, created in, or replaced for or substituted for a portion of the amino acid sequence of the polypeptide derived from the first species,
and the amino acid sequence of the chimeric or recombinant polypeptide is substantially comprised of amino acid sequence derived from the first species,
and the amino acid sequence from the second species when inserted into, joined to, created in, or replaced for or substituted for a portion of the amino acid sequence of the polypeptide derived from the first species generates, forms or creates at least one new epitope on the polypeptide derived from the first species that is capable of generating a humoral antibody response by the first species specific for the at least one new epitope when the chimeric or recombinant polypeptide is administered to the first species,
wherein when the chimeric or recombinant polypeptide is used to generate a humoral immune response from an animal of the first species, the polyclonal antibodies so generated in the first species substantially only specifically bind to the at least one new epitope and do not substantially specifically bind to the polypeptide derived from the first species lacking the at least one new epitope or epitopes created, formed or generated by the at least one heterologous amino acid sequence or amino acid residue derived from the second or additional species inserted into, joined to, created in, or replaced for or substituted for a portion of the polypeptide derived from a first species.

2. The chimeric or recombinant polypeptide of claim 1, wherein:
(a) the polypeptide derived from the second species is a homologue of the polypeptide derived from the first species;
(b) the amino acid sequence from the at least one second species is homologous to the first species, and the at least one homologous second species sequence that is inserted into, joined to, created in, or replaced for or substituted for a portion of the amino acid sequence of the polypeptide derived from the first species replaces all or substantially all of a structurally homologous section or portion of the amino acid sequence of the polypeptide derived from the first species;
(c) the amino acid sequence from the at least one second species is homologous to the first species, and the at least one homologous second species sequence that is inserted into, joined to, created in, or replaced for or substituted for a portion of the amino acid sequence of the polypeptide derived from the first species is structurally homologous to an amino acid sequence of the polypeptide derived from the first species;
(d) a homologue of a first species has at least about 25% to 99% sequence identity to its homologue in the second species;
(e) the homologue of the first species has substantially the same secondary and/or tertiary structure as its homologue in the second species;
(f) a homologue of a first species has at least about 25% to 99% sequence identity to its homologue in the second species and has substantially the same secondary and/or tertiary structure as its homologue in the second species, or, a homologue of a first species has at least about 50% sequence identity to its homologue in the second species, or at least about 70% sequence identity to its homologue in the second species, or at least about 80% sequence identity to its homologue in the second species, or at least about 90% sequence identity to its homologue in the second species; and/or
(g) the first polypeptide and the second polypeptide have a Z score of from about 2 to about 8 when aligned using distance matrix alignment, or the first polypeptide and the second polypeptide have a Z score of at least 8 when aligned using distance matrix alignment.

2. The chimeric or recombinant polypeptide of claim 1, wherein:
(a) the polypeptide derived from the first species and its homologue polypeptide from the second species are antibodies;
(b) the polypeptide derived from the first species and the at least one heterologous amino acid sequence derived from the second species are derived from an antibody heavy chain or an antibody light chain;
(c) the antibody heavy chain is an IgM, IgG, IgA or IgE isotype heavy chain, or the light chain is a kappa or a lambda light chain;
(d) the first species is a mammalian species; the second species is a mammalian species; or, the first species is a species of the order Galliformes or the genus Phasianidae and the second species is a mammalian species;
(e) the first species is a rabbit, a murine species, a sheep, a goat, a pig, a cow a horse or a chicken; and, the second species is a human;
(f) the murine specie is a rat or a mouse;
(g) at least about 80% to about 99% of the amino acid sequence of the chimeric or recombinant polypeptide is amino acid sequence derived from the first species, and/or between about 1% to about 20% of the amino acid sequence of the chimeric or recombinant polypeptide is amino acid sequence derived from the at least one second species;
(h) one, two three, four, five, six, seven or eight or more new epitopes are inserted into, joined to, created in, or replaced for or substituted for a portion of the polypeptide derived from the first species;
(i) the at least one new epitope comprises an epitope derived from a hidden surface of an antibody light chain, wherein the hidden surface is only exposed when the antibody light chain is free and not part of an IgG molecule comprising both light and heavy chains:
(j) the epitope generated, created or formed by the at least one heterologous amino acid sequence derived from the at least one second species is designed by:
(i) aligning the sequence of the polypeptide derived from the first species with its homologue polypeptide from the second species,
(ii) determining one or more amino acid sequence differences between the polypeptide derived from the first species and its homologue polypeptide from the second species,
(iii) selecting at least one amino acid sequence difference between the polypeptide derived from the first species and its homologue polypeptide from the second species, and
(iv) modifying the sequence of the polypeptide derived from the first species to match or be the same as the selected at least one amino acid sequence from the homologue polypeptide of the second species;
(k) selecting at least one amino acid sequence difference between the polypeptide derived from the first species and its homologue polypeptide from the second species comprises highlighting the determined one or more amino acid sequence differences between the polypeptide derived from the first species and its homologue polypeptide from the second species on a three dimensional (3D) model or structure of the polypeptide from the second species, and selecting at least one amino acid sequence difference in or on an exposed or outer surface of the polypeptide;
(l) the amino acid sequence from the at least one second species inserted into, joined to, created in, or replaced for or substituted for a portion of the amino acid sequence of the polypeptide derived from the first species comprises: a sequence present in human IgG3 and not human IgG1, IgG2 or IgG4, or rabbit IgG; a sequence present in human IgG1 and not human IgG2, IgG3 or IgG4, or rabbit IgG; a sequence present in human IgG2 and not human IgG1, IgG3 or IgG4, or rabbit IgG; or, a sequence present in human IgG4 and not human IgG1, IgG2 or IgG3, or rabbit IgG;
(m) the chimeric or recombinant polypeptide is made by a method further comprising removing one or more new epitopes from the at least one heterologous amino acid sequence or amino acid residue derived from the second or additional species after the one or more new epitopes was inserted into, joined to, created in, or replaced for or substituted for a portion of the amino acid sequence of the polypeptide derived from the first species;
(n) at least two or more different heterologous amino acid sequences or amino acid residues are inserted into, joined to, created in, or replaced for or substituted for a portion of the amino acid sequence of the polypeptide derived from the first species;
(o) the at least two or more different heterologous amino acid sequences or amino acid residues are from different animal species;
(p) at least one of the at least two or more different heterologous amino acid sequences or amino acid residues is derived from a human and at least one of the at least two or more different heterologous amino acid sequences or amino acid residues is derived from a non-human or an animal species;
(q) at least one of the heterologous amino acid sequences or amino acid residues comprises an artificial epitope not derived from the at least a second species;
(r) at least one of the heterologous amino acid sequences or amino acid residues comprises an epitope initially derived from the at least a second species that is immunologically silent in the first species (is unable to generate an antibody response in the first species) but is modified to be an immunologically active epitope capable of generating an antibody response against it by the first species;
(s) at least one new epitope in the heterologous amino acid sequences or amino acid residues is modified such that antibodies generated by the first species to the modified new epitope bind less strongly or slower than a comparable unmodified new epitope; and/or
(t) the chimeric or recombinant polypeptide further comprises at least one new epitope derived from an at least second species that is not homologous to the first species, and the at least one new epitope of capable of generating antibodies against it in the first species.

4. A recombinant polypeptide comprising a portion of a first polypeptide from a first species and at least one portion of a second polypeptide from a second species, wherein the at least one portion of the second polypeptide is a homologue of the first polypeptide, and wherein the at least one homologous portion of the second polypeptide comprises an epitope which is not present in the first polypeptide.

5. The recombinant polypeptide of claim 4, wherein:
(a) the portion of the at least one second polypeptide is present at the location of, or substantially at the location of, a homologous portion of the first polypeptide, and has replaced or substantially replaced the homologous portion of the first polypeptide;
(b) the recombinant polypeptide comprises at least a portion of a second polypeptide and at least a portion of a third polypeptide, each being a homologue of different sequences of the first species, and wherein the portion of the second and the portion of the third polypeptide each comprises an epitope which is not present in the first polypeptide;
(c) the first polypeptide and the second polypeptide have similar, or substantially the same, three dimensional structures;
(d) the first polypeptide and the second polypeptide have at least about 25% amino acid identity, or have at least about 50% amino acid identity, or have at least about 70% amino acid identity, or have at least about 90% amino acid identity;
(e) the first polypeptide and the second polypeptide have a Z score of from about 2 to about 8 when aligned using distance matrix alignment, or the first polypeptide and the second polypeptide have a Z score of at least 8 when aligned using distance matrix alignment;
(f) at least one sequence in the first polypeptide is removed and replaced by the homologous portion of the first polypeptide;
(g) the at least one sequence that has been removed from the second polypeptide comprises a sequence that is present in another member of a family from which the first polypeptide and the second polypeptide belong;
(h) the at least one sequence that has been removed comprises a sequence that is present in a domain in another member of a family to which the first polypeptide and the second polypeptide belong;
(i) the at least one sequence that has been replaced comprises an epitope that is specifically recognized by a monoclonal antibody;
(j) the at least one epitope that has been replaced comprises an epitope that results in at least one paratope subtype, and optionally the at least one epitope that has been replaced is a dominant epitope;
(k) the at least one epitope that has been replaced is a weak epitope, or an epitope that elicits a weak humoral response in the first species leading to relatively less titer of antibody;
(l) the epitope in the second polypeptide is modified to reduce the affinity of an antibody generated by the first species an unmodified epitope;
(m) the recombinant polypeptide comprises a portion from a third polypeptide from a third species which comprises an epitope which is not present in the first polypeptide or the second polypeptide;
(n) at least one epitope that is present in another member of a family from which the first polypeptide and the second polypeptide belong has been incorporated into the recombinant polypeptide;
(o) at least one epitope that is present in a domain in another member of a family from which the first polypeptide and the second polypeptide belong is incorporated into the recombinant polypeptide;
(p) the epitope from the second polypeptide is modified to increase the affinity of an antibody which specifically recognizes the epitope from the second polypeptide, or to generate an affinity to the epitope from the second polypeptide by an antibody which specifically recognizes the epitope;
(q) the first species is rabbit and the second species is human, and optionally the first polypeptide is a rabbit antibody light chain constant domain and the second polypeptide is a human antibody light chain constant domain; and/or
(r) when the recombinant polypeptide is administered to the first species, the epitope is capable of generating the production of antibodies which specifically bind to the epitope in the second polypeptide but which do not specifically bind to the first polypeptide.

6. A recombinant nucleic acid encoding a chimeric or recombinant polypeptide as set forth in claim 1.

7. The recombinant nucleic acid of claim 6, wherein:
(a) the recombinant nucleic acid further comprises and is operatively linked to a transcriptional regulatory element, and optionally the transcriptional regulatory element comprises a promoter, and optionally the promoter is an inducible promoter or a constitutive promoter;
(b) the recombinant nucleic acid further comprises sequence encoding an additional protein or peptide moiety or domain, and optionally the additional protein or peptide moiety or domain comprises a purification moiety or domain to aid in the purification or isolation of the chimeric or recombinant antibody encoded by the recombinant nucleic acid, and optionally the additional protein or peptide moiety or domain comprises a histidine (poly-his) tag or a maltose binding protein;
(c) the recombinant nucleic acid further comprises sequence encoding a protease cleavage site positioned between the purification moiety or domain and the sequence encoding the chimeric or recombinant antibody, and optionally the protease cleavage site is a Tobacco Etch Virus (TEV) protease cleavage site;
(d) the recombinant nucleic acid comprises a DNA, an RNA and/or a synthetic or modified nucleotide capable of being recognized by cell machinery to generate proteins;
(e) the nucleic acid comprises a 3′ cap or 3′ methylation, a 5′ and/or a 3′ untranslated region and/or a poly adenine (poly-A) 5′ tail; and/or
(f) the nucleic acid or RNA comprises mRNA.

8. An expression cassette, vector, recombinant virus, artificial chromosome, cosmid or plasmid comprising a recombinant nucleic acid of claim 6.

9. A cell comprising a chimeric or recombinant polypeptide of claim 1, and optionally the cell is a bacterial, fungal, mammalian, yeast, insect or plant cell.

10. A cell comprising an expression cassette, vector, recombinant virus, artificial chromosome, cosmid or plasmid of claim 8, and optionally the cell is a bacterial, fungal, mammalian, yeast, insect or plant cell.

11. A method for generating a polyclonal antibody, or for generating a polyclonal immune serum, that is specific for or specifically binds to an epitope, the method comprising administering to or immunizing a subject with a chimeric or recombinant polypeptide of claim 1,
wherein the subject is the species from which a first polypeptide is derived, and the epitope is derived from the species from which the second polypeptide is derived.

12. The method of claim 11, wherein:
(a) the subject is a mammal or an avian species, or the subject is a rabbit, a murine species, a sheep, a goat, a pig, a cow a horse or a chicken, and optionally the murine specie is a rat or a mouse;
(b) the recombinant or chimeric nucleic acid is an RNA or a DNA construct;
(c) the chimeric or recombinant polypeptide is generated by expressing a recombinant nucleic acid, or an expression cassette, vector, recombinant virus, artificial chromosome, cosmid or plasmid, in a cell, and optionally the cell is a bacterial, fungal, mammalian, yeast, insect or plant cell;
(d) the method further comprises substantially isolating or purifying the chimeric or recombinant polypeptide before the administering to or immunizing the mammal;
(e) the isolating or purifying comprising use of hydrophobic interaction chromatography (HIC), ion exchange chromatography (IEC), size exclusion chromatography (SEC), affinity purification, absorption purification or any combination thereof;
(f) the administering (a), (b) or (c) is repeated between two and twenty times, or is repeated 2, 3, 4, 5, 6, 7, 8, 9 or 10 times, or is repeated at intervals of once every 2 to 20 weeks or 3 to 16 weeks;
(g) the method generates a polyclonal antibody or a polyclonal immune serum that substantially lack antibodies that are not specific for or do not specifically bind to the epitope, and optionally the method generates a polyclonal antibody or a polyclonal immune serum that substantially comprise antibodies that are not specific for or do not specifically bind to a misfolded form of the epitope;
(h) at least one sequence in the first polypeptide is removed and replaced by an epitope formed by a portion of the second polypeptide, and optionally the at least one sequence in the first polypeptide that has been removed is replaced by a sequence comprising an epitope that is present in another member of a family from which the first polypeptide and the second polypeptide belong, and optionally the at least one sequence in the first polypeptide that has been removed is replaced by a sequence comprising an epitope that is present in a domain in another member of a family to which the first polypeptide and the second polypeptide belong, and optionally the at least one sequence in the first polypeptide that has been replaced is replaced by a sequence comprising an epitope that is specifically recognized by a monoclonal antibody;
(i) the at least one sequence in the first polypeptide that has been replaced is replaced by a sequence comprising an epitope that results in at least one paratope subtype, or the at least one sequence in the first polypeptide that has been replaced is replaced by a sequence comprising an epitope that is a dominant epitope;
(j) the at least one sequence in the first polypeptide that has been replaced is replaced by a sequence comprising an epitope that is a weak epitope, or an epitope that elicits a weak humoral response leading to relatively less titer of antibody;
(k) the epitope in the second polypeptide is modified to reduce the affinity of an antibody which specifically recognizes the epitope;
(l) the recombinant polypeptide comprises a portion from a third polypeptide from a third species which comprises an epitope which is not present in the first polypeptide or the second polypeptide;
(m) at least one epitope that is present in another member of a family from which the first polypeptide and the second polypeptide belong has been incorporated into the recombinant polypeptide;
(n) at least one epitope that is present in a domain in another member of a family from which the first polypeptide and the second polypeptide belong is incorporated into the recombinant polypeptide; and/or
(o) the epitope from the second polypeptide is modified to increase the affinity of an antibody which specifically recognizes the epitope from the second polypeptide, or to generate an affinity to the epitope from the second polypeptide by an antibody which specifically recognizes the epitope.

13. A method for generating a polyclonal antibody, or for generating a polyclonal immune serum, that is specific for or specifically binds to an epitope, the method comprising administering to or immunizing a subject with a recombinant polynucleotide of claim 6,
wherein the subject is the species from which a first polypeptide is derived, and the epitope is derived from the species from which the second polypeptide is derived.

14. A chimeric or recombinant polypeptide comprising: a ferritin polypeptide having conjugated or attached thereto by or via a substantially non-immunogenic linker an immunogenic peptide or polypeptide,
wherein the immunogenic peptide or polypeptide comprises a chimeric or recombinant polypeptide as set forth in claim 1, and the ferritin polypeptide is or is derived from the first species.

15. The chimeric or recombinant polypeptide of claim 14, wherein:
(a) the ferritin polypeptide comprises at least one first coiled-coil protein or motif that can bind to a second coiled-coil protein or motif (optionally the second coiled-coil protein or motif comprises or is bound to an immunogenic peptide, optionally covalently attached by a non-immunogenic linker), wherein the first coiled-coil protein or motif is attached to the ferritin polypeptide by a non-immunogenic linker, resulting in a chimeric ferritin-coiled-coil protein polypeptide, which optionally can fold into tertiary structure or a helical bundle structure,
and optionally the coiled-coil protein or motif is derived from the first species, and optionally the coiled-coil protein or motif derived from the first species binds to another coiled-coil protein or motif derived from the first species,
and optionally the ferritin polypeptide comprises two, three, four or more first coiled-coil proteins or motifs,
and optionally the coiled coil protein or motif comprises a gamma-aminobutyric acid type B receptor subunit 1 isoform X1 (GBR1) and/or gamma-aminobutyric acid type B receptor subunit 2 (GBR2)), wherein the GBR1 can selectively bind to GBR2 motif,
and optionally the GBR1 motif comprises:
(SEQ ID NO: 33)
STNNNEEEKSRLLEKENRELEKIIAEKEERVSELRHQLQSR,
and optionally the GBR2 motif comprises:
(SEQ ID NO: 34)
SVNQASTSRLEGLQSENHRLRMKITELDKDLEEVTMQLQDT;
(b) the ferritin polypeptide has inserted into its amino acid sequence at least one His(6)-Lys-His(3) (SEQ ID NO:32) moiety, or a plurality of His(6)-Lys-His(3) (SEQ ID NO:32) moieties;
(c) the substantially non-immunogenic linker comprises a poly-G linker or poly-(GGGGS) linker (SEQ ID NO:31);
(d) the poly-(GGGGS) linker (SEQ ID NO:31) comprises or consists of a (GGGGS)₅ (SEQ ID NO:29) linker;
(e) the non-immunogenic linker is attached to the amino terminus of the ferritin polypeptide;
(f) the first species is a rabbit, or the ferritin polypeptide is derived from a rabbit;
(g) the immunogenic peptide or polypeptide comprises a chimeric immunogenic peptide or polypeptide, and the chimeric immunogenic peptide or polypeptide comprises human immunogenic sequence inserted in a rabbit peptide or polypeptide, and the rabbit polypeptide residues are non-immunogenic when injected into a rabbit; and/or
(h) the non-immunogenic rabbit peptide or polypeptide sequence is derived from a rabbit immunoglobulin polypeptide.

16. A product of manufacture comprising a plurality of chimeric or recombinant polypeptides of claim 14,
and optionally the product of manufacture comprises 24 of the chimeric or recombinant polypeptides,
and optionally each of the chimeric or recombinant polypeptides comprises a coiled-coil protein, and the coiled-coil proteins bind to each other.

17. A method for generating an epitope-specific antibody response in a rabbit, wherein the immune response comprises generation of rabbit antibodies specifically against or that specifically bind to at least one human epitope, and the method comprises administering to a rabbit a sufficient amount of a chimeric or recombinant polypeptide of claim 14, to generate the epitope-specific antibody response.
